(19) United States
(12) Patent Application Publication
(10) Pub. No.: US 2024/0295559 A1
(43) Pub. Date: Sep. 5, 2024

(54) IMMUNOHISTOCHEMISTRY (IHC) PROTOCOLS AND METHODS FOR DIAGNOSING AND TREATING CANCER

(71) Applicants: AGILENT TECHNOLOGIES, INC., Santa Clara, CA (US); ELI LILLY AND COMPANY, Indianapolis,, IN (US)

(72) Inventors: Mark VERARDO, Santa Clara, CA (US); Mariana CAJAIBA, Santa Clara, CA (US); Debra HANKS, Santa Clara, CA (US); Lauren JACOBSON, Santa Clara, CA (US); Gitte NIELSEN, Santa Clara, CA (US); Claudia GOTTSTEIN, Santa Clara, CA (US); Aaron M. GRUVER, Santa Clara, CA (US)

(73) Assignee: AGILENT TECHNOLOGIES, INC., Santa Clara, CA (US)

(21) Appl. No.: 18/025,586

(22) PCT Filed: Sep. 8, 2021
(86) PCT No.: PCT/US2021/049370

§ 371 Date: Mar. 9, 2023

Related U.S. Application Data
(60) Provisional application No. 63/076,079, filed on Sep. 9, 2020.
(60) Provisional application No. 63/114,949, filed on Nov. 17, 2020.

(51) Int. Cl. G01N 33/574; G01N 33/68

(52) U.S. Cl. CPC G01N 33/57415; G01N 33/57484; G01N 33/6875; G01N 2333/4706; G01N 2474/20

(57) ABSTRACT

In alternative embodiments, provided are immunohistochemistry (IHC) methods for determining and scoring reproducibly the extent of nuclear expression of protein Ki-67 (also known as MK167) in a tissue sample. In alternative embodiments, provided are methods for diagnosing, treating or ameliorating or assessing the risk of recurrence for a cancer or a tumor using an IHC method as provided herein. In alternative embodiments, provided are kits comprising components and instructions for practicing methods as provided herein.

### RELATED APPLICATIONS

This Patent Convention Treaty (PCT) International Patent Application claims the benefit of priority under 35 U.S.C. § 119(e) to U.S. Provisional Patent Application Ser. No. 63/076,079, Sep. 9, 2020; and U.S. Ser. No. 63/114,949, Nov. 17, 2020. The aforementioned applications are expressly incorporated herein by reference in their entirety and for all purposes.

### TECHNICAL FIELD

This invention generally relates to cancer treatments, companion or complementary diagnostics and immunohistochemical methods. In alternative embodiments, provided are immunohistochemistry (IHC) methods for determining and scoring reproducibly the extent of nuclear expression of protein Ki-67 (also known as MK167) in a tissue sample. In alternative embodiments, provided are methods for diagnosing, treating or ameliorating or assessing the risk of recurrence for a cancer or a tumor using an IHC method as provided herein. In alternative embodiments, provided are kits comprising components and instructions for practicing methods as provided herein. The present application describes methods for scoring Ki-67 expression and utilizing the score as a companion or complementary diagnostic or to treat or ameliorate cancer or a tumor.

### BACKGROUND

Ki-67 antigen (also known as antigen identified by monoclonal antibody Ki-67) is a nuclear protein expressed during all active phases of the mammalian cell cycle (G1, S, G2, and M-phases) and downregulated in resting cells (G0-phase). During interphase, the antigen can be exclusively detected within the nucleus, whereas in mitosis most of the protein is relocated to the surface of the chromosomes. Localization of Ki-67 antigen correlates with distinct functions. During interphase, Ki-67 is required for normal cellular distribution and nucleolar association of heterochromatin. During mitosis, Ki-67 plays a role in the formation of the perichromosomal layer and prevents aggregation of mitotic chromosomes. The antigen is rapidly degraded as the cell enters the non-proliferative state and there appears to be no expression of Ki-67 as detected by immunohistochemistry (IHC) during DNA repair processes.

### SUMMARY

In alternative embodiments, provided are immunohistochemistry (IHC) methods for determining and scoring the extent of nuclear expression of protein Ki-67 (also known as MKI67) in a tissue sample, the method comprising:
(a) staining a tissue sample with an antibody which specifically binds to Ki-67; and
(b) determining the total number of viable invasive tumor or cancer cells having anti-Ki-67 nuclear staining, and determining the total number of staining and non-staining viable invasive tumor or cancer cells in at least a portion of the tissue sample, wherein an invasive tumor or cancer cell is counted as positively stained with anti-Ki-67 if there is convincing and complete nuclear anti-Ki-67 staining, and if the invasive tumor or cancer cell displays at an anti-Ki-67 nuclear staining at any intensity above a defined threshold, and
(c) determining a Ki-67 score (%), wherein the Ki-67 score (%) is the number of Ki-67 staining viable invasive tumor or cancer cells found in the tissue sample divided by the total number of staining and non-staining viable invasive tumor or cancer cells, multiplied by 100.

In alternative embodiments of IHCs method as provided herein:
it is determined if the tissue section is or is not adequate for the determining and scoring of the amount of nuclear expression of protein Ki-67, and a tissue section is considered adequate for evaluation if about 100 or more Ki-67 stained viable invasive tumor or cancer cells are present;
the Ki-67 score (%) is the number of Ki-67 staining viable invasive tumor or cancer cells found in the tissue sample divided by the total number of staining and non-staining viable invasive tumor or cancer cells, multiplied by 100;
an invasive tumor or cancer cell is counted as positively stained with anti-Ki-67 if there is convincing and complete nuclear anti-Ki-67 staining, and if the invasive tumor or cancer cell displays at an anti-Ki-67 nuclear staining at any intensity of 1+ or higher;
a section or portion of the tissue sample is prepared on a slide or equivalent, and the section or portion of the tissue sample is stained on the slide;
the antibody which specifically binds to Ki-67 comprises a monoclonal mouse anti-Ki-67 antibody;
the anti-Ki-67 comprises monoclonal mouse anti-Ki-67 clone MIB-1 (available from Agilent Technologies, Inc., Santa Clara, CA);
the anti-Ki-67 comprises a substantially isolated or substantially purified monoclonal mouse anti-Ki-67 clone MIB-1k;
the total number of Ki-67 stained viable invasive tumor or cancer cells is evaluated under high magnification,
the high magnification is at least about 10× magnification, or is between about 10× and 40× magnification, or is between about 10× and 60× magnification;
the invasive tumor or cancer cell is counted as positively stained with anti-Ki-67 if there is convincing and complete nuclear anti-Ki-67 staining and the invasive tumor cell and cancer cell is counted at an anti-Ki-67 nuclear staining at any intensity 1+ and higher;
there is convincing and complete nuclear anti-Ki-67 staining, and at an anti-Ki-67 nuclear staining at any intensity 1+ and higher, when:
(a) the staining signal is unequivocally brown, or
(b) the staining corresponds to a nucleus, or
(c) the staining covers the whole chromatin distribution within the nucleus, or
(d) cells that exhibit a grey coloring in the nucleus are considered not stained with anti-Ki-67, or
(e) any combination of two or more of (a) to (d);
   there is convincing and complete nuclear anti-Ki-67 staining, and at an anti-Ki-67 nuclear staining at any intensity 1+ and higher, when: (a) the staining signal is unequivocally brown, (b) the staining corresponds to a nucleus, (c) the staining covers the whole chromatin distribution within the nucleus, and (d) cells that exhibit a grey coloring in the nucleus are considered not stained with anti-Ki-67;
   the methods further comprise excluding from the calculation of the Ki-67 score (%): tumor cells or cancer cells with only cytoplasmic or membrane staining; non-invasive neoplasia or carcinoma in situ cells, non-viable or necrotic tumor or cancer cells, apoptotic nuclei or nuclear debris, tumor or cancer cells in poorly preserved tissue areas, benign epithelial cells, non-neoplastic cells and/or lymphocytes with nuclear staining, apoptotic cells, necrotic cells, cells not exhibiting an intended color, cells in which a stain reflecting of binding of the antibody to Ki-67 is not present throughout the entire chromatin distribution in the nucleus, cells exhibiting membrane staining, cells exhibiting cytoplasmic staining, lymphocytes, and stromal cells;
   cells on an edge of a tissue sample specimen are not scored if staining due to an edge artifact is inconsistent with the rest of the tissue sample specimen;
   in step (b) determining if the tissue section is or is not adequate for the determining and scoring of the amount of nuclear expression of protein Ki-67, wherein one parameter considered is: a tissue section is adequate for evaluation if about 200 or more viable invasive tumor cells are present;
   in step (d): (i) if the Ki-67 Score (%) is less than (<) 20%, then the tissue sample is determined to have diagnostic negative Ki-67 expression; and, (ii) if the Ki-67 Score (%) is greater than or equal to (≥) 20%, then the tissue sample is determined to have diagnostic positive Ki-67 expression;
   the tissue sample comprises a formalin-fixed, paraffin-embedded (FFPE) specimen; or the section of the tissue sample is prepared by a protocol comprising fixation in about 10% neutral buffered formalin for between about 6 to 72 hours;
   the tumor or cancer is a or breast cancer or breast carcinoma, a head and neck cancer, a colorectal cancer, a bladder cancer, a lung cancer, a gastrointestinal stromal tumor (GIST), a prostate cancer, a cervical cancer, or a renal cell carcinoma, or, the tumor or cancer is an invasive or metastatic breast carcinoma or breast cancer; and/or
   the tissue sample is a biopsy sample, or the tissue sample is or is derived from a needle biopsy sample, or the tissue sample is or is derived from a fine-needle aspirate, a cytology specimen or a bone decalcification.

In alternative embodiments, provided are methods for treating or ameliorating a cancer or a tumor in a patient, comprising determining and scoring the amount of nuclear protein Ki-67 (also known as MKI67) in a tissue sample from a patient in need thereof using an IHC method as provided herein, wherein if the tissue sample is determined or scored to have a high or a diagnostic positive Ki-67 expression, the patient is treated with a cancer therapeutic to which the patient is likely to respond favorably. In alternative embodiments, the tumor or cancer is a breast carcinoma or breast cancer, a head and neck cancer, a colorectal cancer, a bladder cancer, a lung cancer, a gastrointestinal stromal tumor (GIST), a prostate cancer, a cervical cancer, or a renal cell carcinoma. In alternative embodiments, the breast carcinoma or breast cancer is an early stage breast cancer or breast carcinoma. In alternative embodiments, the breast carcinoma or breast cancer is an invasive or metastatic breast carcinoma or breast cancer.

In alternative embodiments, provided are methods for treating or ameliorating a cancer or a tumor in an individual in need thereof, comprising: (a) determining if cells in a tissue sample from the individual in need thereof have a low or a high, or a negative or a diagnostic positive, Ki-67 expression score (%), as determined by a protocol comprising use of an immunohistochemistry (IHC) method as provided herein; and, (b) administering a tumor or a cancer therapeutic to the individual if the tissue sample is found to have a high or a diagnostic positive Ki-67 expression score (%). In alternative embodiments, the cancer or tumor is a breast carcinoma, or the cancer or tumor is an early stage breast cancer, or the cancer or tumor is an invasive or metastatic breast carcinoma. In alternative embodiments, the treating or amelioration of the cancer or tumor comprises administering to the individual in need thereof a pharmaceutical formulation comprising an ATP competitive inhibitor of a cyclin-dependent kinase. In alternative embodiments, the pharmaceutical formulation comprises an ATP competitive inhibitor of cyclin-dependent kinases 4 and 6. In alternative embodiments, the ATP competitive inhibitor of cyclin-dependent kinases 4 and 6 comprises abemaciclib. In alternative embodiments, the treating or amelioration of the cancer or tumor comprises administering to the individual in need thereof a pharmaceutical formulation comprising palbociclib (optionally IBRANCE™ or PALBONIX™), or ribociclib (optionally KISQUALI™). In alternative embodiments, the treating or amelioration of the cancer comprises administering to the individual in need thereof a pharmaceutical formulation comprising an ATP competitive inhibitor of a cyclin-dependent kinase in combination with endocrine therapy. In alternative embodiments, the individual in need thereof is a patient with a breast cancer, or the individual in need thereof is a patient with early stage breast cancer, or the individual in need thereof is a patient with node-positive, early stage, resected hormone receptor positive (HR+), human epidermal growth factor receptor 2 negative (HER2−) breast cancer at high risk of recurrence.

In alternative embodiments, provided are methods for treating or ameliorating a breast cancer in a breast cancer patient, comprising determining if cells in a tissue sample have a low or high, or diagnostic negative or diagnostic positive, Ki-67 expression score (%) as determined by a protocol comprising use of an immunohistochemistry (IHC) method as provided herein, and administering a cancer therapeutic if the Ki-67 expression score (%) is high or diagnostic positive. In alternative embodiments, the breast cancer patient has a hormone receptor positive (HR+) breast cancer after surgery. In alternative embodiments, the breast cancer patient receives adjuvant endocrine therapy.

In alternative embodiments, provided are methods for assessing the extent of Ki-67 expression in a tumor or a cancer comprising: contacting a tissue sample or a portion thereof from an individual having a tumor or a cancer with an antibody or portion thereof which specifically binds to Ki-67; and, determining a Ki-67 score (%) by dividing the number of Ki-67 staining viable tumor or cancer cells specifically bound by the antibody with the total number of staining and non-staining viable cancer or tumor cells and multiplying the result by 100, thereby obtaining the Ki-67 score (%).

In alternative embodiments of methods for assessing the extent of Ki-67 expression in a tumor or a cancer:
the cancer or tumor cells are viable invasive breast carcinoma cells;
the methods further comprise determining whether the Ki-67 score (%) is 10 or above, or further comprise determining whether the Ki-67 score (%) is 20 or above;
determining the number of Ki-67 positive viable tumor or cancer cells comprises determining the number of viable tumor or cancer cells having Ki-67 staining in the nucleus;
determining the number of Ki-67 positive viable tumor or cancer cells comprises determining the number of viable tumor or cancer cells having Ki-67 staining throughout the entire chromatin distribution in the nucleus;
the methods further comprise determining whether a color reflecting binding of the antibody to Ki-67 is the intended color, and optionally the intended color is brown, and optionally the brown color is generated by staining with 3,3′-Diaminobenzidine (DAB);
the methods further comprise excluding from the Ki-67 positive tumor or cancer cells at least one type of cell selected from the group consisting of: tumor cells or cancer cells with only cytoplasmic or membrane staining; non-invasive neoplasia or carcinoma in situ cells, non-viable or necrotic tumor or cancer cells, apoptotic nuclei or nuclear debris, tumor or cancer cells in poorly preserved tissue areas, benign epithelial cells, non-neoplastic cells and/or lymphocytes with nuclear staining, apoptotic cells, necrotic cells, cells not exhibiting an intended color, cells in which a stain reflecting of binding of the antibody to Ki-67 is not present throughout the entire chromatin distribution in the nucleus, cells exhibiting membrane staining, cells exhibiting cytoplasmic staining, lymphocytes, and stromal cells;
the methods further comprise excluding from the portion of the tissue sample from which the Ki-67 score (%) is determined portions of the tissue sample exhibiting at least one artifact selected from the group consisting of distorted morphology, poor fixation, crush and cautery artifacts;
the Ki-67 score (%) is calculated in a portion of the tissue sample comprising at least 100 cells, or, the Ki-67 score (%) is calculated in a portion of the tissue sample comprising at least 200 cells;
the methods further comprise administering a cancer therapeutic based on the Ki-67 score (%);
the cancer therapeutic is an ATP competitive inhibitor of a cyclin-dependent kinase, and optionally the ATP competitive inhibitor of a cyclin-dependent kinase is abemaciclib, and optionally the cancer therapeutic comprises palbociclib (optionally IBRANCE™ or PALBONIX™), or ribociclib (optionally KISQUALI™);
the tissue sample is from a patient with node-positive, early, resected hormone receptor positive (HR+), human epidermal growth factor receptor 2 negative (HER2−) breast cancer;
the methods further comprise determining that a subject is likely to respond favorably to treatment with a cancer therapeutic if the Ki-67 score (%) is above a threshold value, and optionally the threshold value is about 1%, about 5%, about 10%, about 20%, about 30%, about 40%, about 50% or about any number between 1% and 50%;
the cancer therapeutic is a cyclin dependent kinase inhibitor, and optionally the cyclin dependent kinase inhibitor is a CDK4 or CDK6 inhibitor, and optionally the cyclin dependent kinase inhibitor is abemaciclib, palbociclib (optionally IBRANCE™ or PALBONIX™), or ribociclib (optionally KISQUALI™);
the cancer is a breast cancer or breast carcinoma, a head and neck cancer, a colorectal cancer, a bladder cancer, a lung cancer, gastrointestinal stromal tumors (GIST), prostate cancer, cervical cancer, or renal cell carcinoma, and optionally the cancer is metastatic breast cancer; and/or
the methods further comprise administering the cancer therapeutic if the Ki-67 score (%) is above the threshold value, and optionally the methods further comprise administering a treatment other than a cyclin dependent kinase inhibitor if the Ki-67 score (%) is below the threshold value.

In alternative embodiments, provided are kits comprising an antibody which specifically binds to Ki-67, and scoring guidelines comprising a method as provided herein; and optionally the kits comprise scoring guidelines comprising (or as described in) a method as provided herein. In alternative embodiments, the kits further comprise images indicating a plurality of Ki-67 staining levels. In alternative embodiments, the kits further comprise images depicting staining of the whole nuclear chromatin distribution.

The details of one or more exemplary embodiments of the invention are set forth in the accompanying drawings and the description below. Other features, objects, and advantages of the invention will be apparent from the description and drawings, and from the claims.

All publications, patents, patent applications cited herein are hereby expressly incorporated by reference in their entireties for all purposes.

### DESCRIPTION OF DRAWINGS

The patent or application file contains at least one drawing executed in color. Copies of this patent or patent application publication with color drawing(s) will be provided by the Office upon request and payment of the necessary fee.

The drawings set forth herein are illustrative of exemplary embodiments provided herein and are not meant to limit the scope of the invention as encompassed by the claims.

Figures are described in detail herein.

Like reference symbols in the various drawings indicate like elements.

### DETAILED DESCRIPTION

In alternative embodiments, provided are immunohistochemistry (IHC) methods for determining and scoring reproducibly the extent of nuclear expression of protein Ki-67 (also known as MKI67) in a tissue sample such as a biopsy. In alternative embodiments, IHC methods as provided herein provide a reliable, standardized, reproducible and harmonized methodology for assessing the extent of nuclear expression of protein Ki-67, thus creating greater between-laboratory and between-study comparability, and allowing earlier valid applications of Ki-67 in clinical practice. In alternative embodiments, IHC methods as provided herein provide for high quality staining and reliable diagnostic assessment.

In alternative embodiments, methods and compositions described herein are utilized as an aid in identifying patients with early breast cancer at high risk of recurrence for whom treatment with abemaciclib, palbociclib optionally IBRANCE™ or PALBONIX™), or ribociclib (optionally KISQUALI™) or other cyclin dependent kinase inhibitors is being considered in combination with standard adjuvant endocrine therapy. Methods as provided herein allow pathologists and laboratory personnel to achieve accurate and reproducible results in assessing Ki-67 expression in formalin fixed, paraffin-embedded (FFPE) breast carcinoma specimens. In alternative embodiments, methods as provided herein provide a Ki-67 expression evaluation that can be used to identify patients eligible for a cancer treatment. In some embodiments, the cancer or tumor may be a breast cancer or carcinoma (see for example, Dowsett, M et al., JNCI, Vol. 103, Issue 2, Nov. 16, 2011), a head and neck cancer (see for example, Ahmed et al., Int J Biol Markers. 2016 May 28; 31(2):193-203), a colorectal cancer (see for example, Li et al., Mol Med Rep. 2015 March, 11(3):1566-72), a bladder cancer (see for example, He et al., BMJ Open. 2018 Apr. 17; 8 (4):e019635), a lung cancer (see for example, Wei et al., 2018, Respir Res. August 13; 19(1):150), gastrointestinal stromal tumors (GIST) (see for example, Zhou et al., 2017 Medicine (Baltimore) August; 96 (34):e7911), prostate cancer, (see for example, Berlin et al., 2017, Urol Oncol. August; 35(8):499-506), a cervical cancer (see for example, Silva et al., 2017, Pathol Res Pract. July; 213(7):723-729) or a renal cell carcinoma (see for example, Xie et al., 2017 Sci Rep. March 13; 7:44281). In some embodiments the methods may be used for treatment or amelioration of breast cancer by administration of the drugs abemaciclib, palbociclib (optionally IBRANCE™ or PALBONIX™), or ribociclib (optionally KISQUALI™) or other cyclin dependent kinase inhibitors, such as CDK4 or CDK6 inhibitors.

In alternative embodiments, provided are methods for diagnosing and treating or ameliorating a cancer of a tumor such as a breast cancer or breast carcinoma, a head and neck cancer, a colorectal cancer, a bladder cancer, a lung cancer, gastrointestinal stromal tumors (GIST), prostate cancer, a cervical cancer, or a renal cell carcinoma, comprising use of an IHC method as provided herein. In alternative embodiments, provided are kits comprising components and instructions for practicing IHC methods as provided herein.

In breast cancer, immunohistochemical (IHC) assessment of the proportion of cells staining for the nuclear antigen Ki67 is the most widely used method for comparing proliferation between tumor samples. Ki67 is measured in these scenarios for clinical research, including as a primary efficacy endpoint for clinical trials, or for clinical management. At present, the enormous variation in analytical practice markedly limits the value of Ki67 in each of these contexts. For example, 17 of the 18 studies that included more than 200 patients showed statistically significant association between Ki67 and prognosis providing compelling evidence for a biological relationship, but the cutoffs to distinguish “Ki67 high” from “Ki67 low” varied from 1% to 28.6%, thereby severely limiting its clinical utility (see for example, Urruticoechea et al J Clin Oncol. 2005; 23(28):7212-7220). Detection of Ki-67 has also been described in Dowsett, M et al., JNCI, Vol. 103, Issue 2, Nov. 16, 2011, Leung et al., npj Breast Cancer, May 18, 2016, T Harris L N, Ismaila N, McShane L M, Hayes D F. Use of Biomarkers to Guide Decisions on Adjuvant Systemic Therapy for Women With Early-Stage Invasive Breast Cancer American Society of Clinical Oncology Clinical Practice Guideline Summary. J Oncol Pract 2016; 12(4):384-9 doi: 10.1200/JOP.2016.010868 (published Online First: Epub Date), Polley M Y, Leung S C, McShane L M, et al. An international Ki67 reproducibility study. J Natl Cancer Inst 2013; 105(24):1897-906 doi: 10.1093/jnci/djt306), Polley M Y, Leung S C, Gao D, et al. An international study to increase concordance in Ki67 scoring. Mod Pathol 2015; 28(6):778-86 doi: 10.1038/modpathol.2015.38. Despite extensive effort spent on evaluating Ki67 as a prognostic and/or predictive marker in the past three decades, this biomarker is still not completely integrated into clinical decision making mainly because of the lack of standardization and reproducibility in staining techniques and Ki67 scoring methods.

Ki-67 is a nuclear stain. One of the issues previously encountered by pathologists and scores with nuclear stains is defining and identifying the lower limit of positivity. For example, the ASCO/CAP guidelines for Estrogen Receptor and Progesterone Receptor (Hammond M E. et al, Arch Pathol Lab Med, Vol 134, July 2010) state that intensity should be reported and can be used as a way to measure assay quality over time. However, they do not give guidance on how to evaluate this intensity, except for defining three buckets that intensity should be reported as, strong, moderate, weak. Further, there is no definition of how to define the lower limit of positivity; namely what is positive (or diagnostic positive) and what is negative (or diagnostic negative). In the case of cells at the lower limit of detection, anecdotally called grey cells, it can be difficult for scorers to make a determination of what is a positive cell or a negative cell. This can be true for any nuclear stain where there is a dynamic range of the assay that approaches the level of detection by a human observer. The scoring methods as provided herein define this lower limit of detection by the presence of staining and by where the staining occurs (on a cellular level); thus, the difficulties associated with intensity determination (the level of staining) are converted to the questions of where the staining occurs.

Some embodiments relate to a method of identifying a subject who is likely to respond favorably to treatment with a cancer therapeutic, such as a cyclin dependent kinase inhibitor, by calculating a Ki-67 score (%) and determining whether the score (%) is above a threshold value. In some embodiments, the Ki-67 score (%) is calculated using the equation:

$$Ki - 67 score ( \% ) = \frac{\# of Ki - 67 staining viable invasive tumor cells}{Total \# of staining and non - staining viable invasive tumor cells} \times 100$$

In some embodiments, the threshold value is about 1%, about 5%, about 100%, about 20%, about 30%, about 40%, about 50% or about any number between 1% and 50%. In some embodiments, the cyclin dependent kinase inhibitor is abemaciclib, palbociclib (optionally IBRANCE™ or PALBONIX™), or ribociclib (optionally KISQUALI™). In some embodiments, the cancer is a breast cancer or breast carcinoma, a head and neck cancer, a colorectal cancer or a bladder cancer, a lung cancer, gastrointestinal stromal tumors (GIST), prostate cancer, cervical cancer, or renal cell carcinoma. In some embodiment, the cancer is metastatic breast cancer. In some embodiments, the method further comprises administering the cancer therapeutic if the Ki-67 score (%) is above the threshold value. In some embodiments, the method further comprises administering a treatment other than a cyclin dependent kinase inhibitor if the Ki-67 score (%) is below the threshold value.

In alternative embodiments, when using IHC methods as provided herein and the exemplary scoring guidelines as provided herein the lower limit of positivity is defined by the staining of the cell and the presence or absence of staining, rather than being defined by the lower limit of positivity. In alternative embodiments, IHC methods as provided herein comprise use of an exemplary series of rules that define what is staining and asks the scorer/pathologist where that staining is.

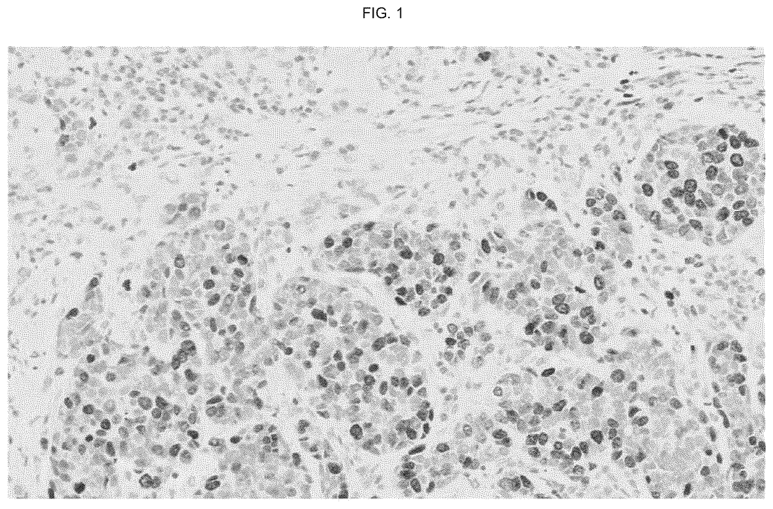

In alternative embodiments, methods and kits as provided herein are used for in vitro diagnostic uses. In alternative embodiments, the Ki-67 IHC as provided herein is an immunohistochemical (IHC) assay using an anti-Ki-67 antibody such as the monoclonal mouse anti-Ki-67 clone MIB-1, in the detection of a Ki-67 protein in formalin-fixed, paraffin-embedded (FFPE) tissue samples such as breast carcinoma tissue samples. In alternative embodiments, the EnVision FLEX™ visualization system on Dako OMNIS™ is used. FIG. 1 illustrates an FFPE breast carcinoma tissue stained with a Ki-67 IHC method as provided herein using the monoclonal mouse anti-Ki-67 clone MIB-1, as visualized with 10×magnification.

In alternative embodiments, the Ki-67 IHC as provided herein is used as an aid in identifying patients with a tumor or a cancer, for example, an early cancer such as breast cancer at high risk of recurrence, and for breast cancer patients, for whom abemaciclib, palbociclib (optionally IBRANCE™ or PALBONIX™), or ribociclib (optionally KISQUALI™) or other cyclin dependent kinase inhibitors, such as CDK4 or CDK6 inhibitors, treatment is being considered in combination with standard adjuvant endocrine therapy.

In alternative embodiments, the patient population considered for abemaciclib, palbociclib (optionally IBRANCE™ or PALBONIX™), or ribociclib (optionally KISQUALI™) or other cyclin dependent kinase inhibitors, such as CDK4 or CDK6 inhibitors, in the adjuvant setting, in combination with endocrine therapy, are patients with node-positive, early, resected hormone receptor positive (HR+), human epidermal growth factor receptor 2 negative (HER2−) breast cancer at high risk of recurrence.

In alternative embodiments, the Companion Diagnostic Indications are:

| | Exemplary Ki-67 IHC | |
|---|---|---|
| Tumor Indication | Cut-off | Intended Use |
| Invasive Breast | ≥20% of invasive tumor | The exemplary Ki-67 |
| Carcinoma | cells positive | IHC is indicated as an |
| | | aid in identifying breast |
| | | cancer patients for |
| | | treatment with |
| | | abemaciclib |

In alternative embodiments, the Ki-67 IHC as provided herein is an immunohistochemical assay, where Ki-67 expression in a cancer or a tumor, for example, a breast cancer or a breast carcinoma, a head and neck cancer, a colorectal cancer, a bladder cancer, a lung cancer, a gastrointestinal stromal tumor (GIST), prostate cancer, cervical cancer, or renal cell carcinoma, is determined by using the exemplary Ki-67 score (%) as provided herein, which is the number of Ki-67 staining viable invasive tumor cells divided by the total number of viable invasive tumor cells, multiplied by 100. In alternative embodiments, cancer or tumor, for example, breast cancer or breast carcinoma tissue, specimens that are tested for Ki-67 expression are scored and divided into Ki-67 expression levels based on the whole tissue score:

$$Ki - 67 Score ( \% ) less than ( < ) 20 \% : Low Ki - 67 expression , or a diagnostic negative .$$

$$Ki - 67 Score ( \% ) greater than or equal to ( \geq ) 20 \% : High Ki - 67 expression , or a diagnostic positive .$$

In alternative embodiments, Ki-67 expression levels are used to inform patient eligibility for treatment with a cancer drug, for example, for treatment for breast cancer with the drug abemaciclib, palbociclib (optionally IBRANCE™ or PALBONIX™), or ribociclib (optionally KISQUALI™) or other cyclin dependent kinase inhibitors, such as CDK4 or CDK6 inhibitors.

### Products of Manufacture and Kits

Provided are products of manufacture and kits for practicing methods as provided herein, including for example, monoclonal mouse anti-Ki-67 antibodies such as clone MIB-1 anti-Ki-67 antibodies, and/or reagents for practicing IHC, including for example reagents as described herein, see Example 1, below; and optionally, products of manufacture and kits can further comprise instructions for practicing methods as provided herein.

Any of the above aspects and embodiments can be combined with any other aspect or embodiment as disclosed here in the Summary, Figures and/or Detailed Description sections.

As used in this specification and the claims, the singular forms “a,” “an” and “the” include plural referents unless the context clearly dictates otherwise.

Unless specifically stated or obvious from context, as used herein, the term “or” is understood to be inclusive and covers both “or” and “and”.

Unless specifically stated or obvious from context, as used herein, the term “about” is understood as within a range of normal tolerance in the art, for example within 2 standard deviations of the mean. About (use of the term “about”) can be understood as within 25%, 24%, 23%, 22%, 21%, 20%, 19%, 18%, 17%, 16%, 15%, 14%, 13%, 12% 11%, 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Unless specifically stated or obvious from context, as used herein, the terms “substantially all”, “substantially most of”, “substantially all of” or “majority of” encompass at least about 90%, 95%, 97%, 98%, 99% or 99.5%, or more of a referenced amount of a composition.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents. Incorporation by reference of these documents, standing alone, should not be construed as an assertion or admission that any portion of the contents of any document is considered to be essential material for satisfying any national or regional statutory disclosure requirement for patent applications. Notwithstanding, the right is reserved for relying upon any of such documents, where appropriate, for providing material deemed essential to the claimed subject matter by an examining authority or court.

Modifications may be made to the foregoing without departing from the basic aspects of the invention. Although the invention has been described in substantial detail with reference to one or more specific embodiments, those of ordinary skill in the art will recognize that changes may be made to the embodiments specifically disclosed in this application, and yet these modifications and improvements are within the scope and spirit of the invention. The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. Thus, the terms and expressions which have been employed are used as terms of description and not of limitation, equivalents of the features shown and described, or portions thereof, are not excluded, and it is recognized that various modifications are possible within the scope of the invention. Embodiments of the invention are set forth in the following claims.

The invention will be further described with reference to the examples described herein; however, it is to be understood that the invention is not limited to such examples.

### EXAMPLES

Unless stated otherwise in the Examples, all recombinant DNA techniques are carried out according to standard protocols, for example, as described in Sambrook et al. (2012) Molecular Cloning: A Laboratory Manual, 4th Edition, Cold Spring Harbor Laboratory Press, NY and in Volumes 1 and 2 of Ausubel et al. (1994) Current Protocols in Molecular Biology, Current Protocols, USA. Other references for standard molecular biology techniques include Sambrook and Russell (2001) Molecular Cloning: A Laboratory Manual, Third Edition, Cold Spring Harbor Laboratory Press, NY, Volumes I and II of Brown (1998) Molecular Biology LabFax, Second Edition, Academic Press (UK).

### Example 1: Exemplary Methods and Kits as Provided Herein

In alternative embodiments, provided are methods for assessing the extent of Ki-67 expression in a tumor or a cancer comprising contacting a tissue sample or a portion thereof from an individual having a tumor or a cancer with an antibody or portion thereof which specifically binds to Ki-67, and determining a Ki-67 score (%), which is the number of Ki-67 staining viable tumor or cancer cells specifically bound by the antibody divided by the total number of staining and non-staining viable cancer or tumor cells and multiplying the result by 100 to obtain the Ki-67 score (%).

In one embodiment, an exemplary Ki-67 IHC as provided herein is a modular assay configured for the Dako OMNIS™ workflow and contains optimized, purified primary antibody and Negative Control Reagent (NCR) ready to use (RTU). Deparaffinization, rehydration, and target retrieval are performed onboard the Dako OMNIS™ automated staining instrument using a two-step incubation of CLEARIFY™, followed by Low pH Target Retrieval Solution (TRS). The specimens are then incubated with Monoclonal Antibody to Ki-67 or the NCR prior to Peroxidase Block. Following incubation with the primary antibody/NCR and Peroxidase Block, specimens are incubated with a ready-to-use visualization reagent consisting of secondary antibody molecules and horseradish peroxidase molecules coupled to a dextran polymer backbone. The enzymatic conversion of the subsequently added chromogen (for example, 3,3′-Diaminobenzidine (DAB) chromogen) results in precipitation of a visible reaction product at the site of antigen. The specimen may then be counterstained and cover slipped. Results are interpreted using a bright-field microscope. Ki-67 IHC as provided herein can be used in automated staining, for example, using the Dako OMNIS™ instrument.

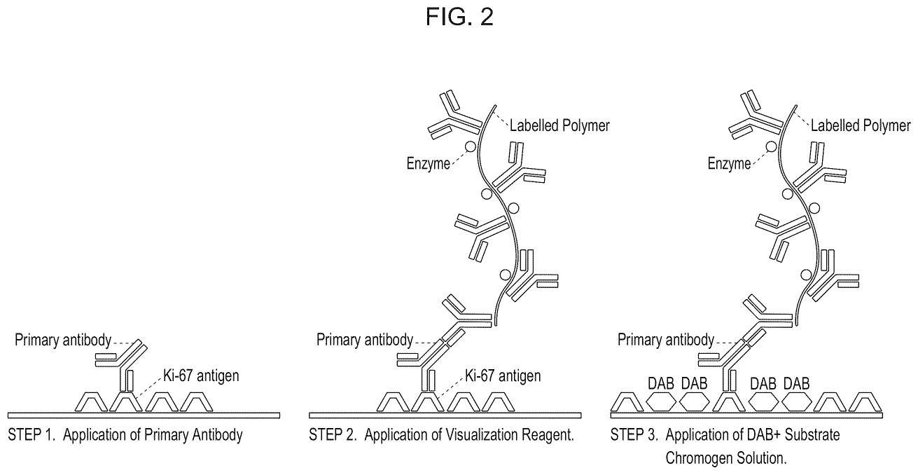

FIG. 2 schematically illustrates an exemplary staining procedure.

In one embodiment of an exemplary Ki-67 IHC configured for Dako OMNIS™ workflow, the following reagents are required for multiple individual runs:
1 Primary Antibody: Purified Monoclonal Mouse Anti-Ki-67, Clone MIB-1 RTU
2 Negative Control Reagent RTU
The following Dako OMNIS™ bulk and visualization reagents are also required: EnVision FLEX, High pH (Dako OMNIS™) (Code GV800) or EnVision Mini Kit, High pH (Dako OMNIS™), containing: EnVision FLEX DAB+ Chromogen (Dako OMNIS™)
EnVision FLEX™ Peroxidase-Blocking Reagent (Dako OMNIS™)
EnVision FLEX™ Substrate Buffer (Dako OMNIS™)
EnVision FLEX™ Visualization Reagent (Dako OMNS™)
EnVision FLEX™ Target Retrieval Solution, Low pH, (50×) (Dako OMNIS™)
Hematoxylin (Dako OMNIS™ s)
Wash Buffer (20×) (Dako OMNIS™)
CLEARIFY™ Clearing Agent (Dako OMNIS™)
Dako OMNIS™ Sulfuric Acid, 0.3 M.

In an exemplary protocol: specimens must be handled to preserve the tissue for immunohistochemical staining; intact tumor morphology is to be determine and the presence of sufficient tumor cells evaluated. Standard methods of tissue processing are used for all specimens.

### Exemplary Specimen Preparation Protocols

In alternative embodiments, specimens are handled to preserve the tissue for IHC staining. Standard methods of tissue processing should be used for all specimens.

### Exemplary Paraffin-Embedded Tissue Preparation

In alternative embodiments, formalin-fixed, paraffin-embedded (FFPE) tissues are suitable for use; however, alternative fixatives have not been validated and may give erroneous results. In alternative embodiments, fixation time for between about 6 to 72 hours in 10% neutral buffered formalin (NBF) is used. Fixation time of less than 6 hours may result in variable Ki-67 detection. In alternative embodiments, ischemia times are kept at one hour or less. In alternative embodiments, specimens are blocked into a thickness of 3 or 4 mm, fixed in formalin and dehydrated and cleared in a series of alcohols and xylene, followed by infiltration with melted paraffin. In alternative embodiments, paraffin temperature does not exceed 60° C.

### Exemplary Tissue Sections Preparation

In alternative embodiments, FFPE tissue specimens are cut into sections of 4-5 μm. In alternative embodiments, after sectioning, tissues are mounted on Dako FLEX IHC Microscope Slides (Code K8020) or SuperFrost Plus microscope slides, and then placed in a 58±2° C. calibrated oven for 1 hour.

In alternative embodiments, to preserve antigenicity, tissue sections mounted on slides are stained within 5 months of sectioning when held in the dark at 2 to 8° C. (preferred), or can be stained within 4 months when held at about room temperature or 25° C. In alternative embodiments, slide storage and handling conditions do not exceed 25° C. at any point post-mounting to ensure tissue integrity and antigenicity.

In alternative embodiments, the tissue specimens are mounted on the slide within the defined slide staining area. The Dako OMNIS™ Basic User Guide can be used to determine the dimensions of the slide staining area.

### Exemplary Reagent Preparation

In alternative embodiments, EnVision FLEX Target Retrieval Solution, Low pH (50×) (GV805) and Wash Buffer (20×) (GC807) is diluted to 1×concentration according to their Instructions for Use. The color of the FLEX Target Retrieval Solution, Low pH (50×) is red.

In alternative embodiments, the pH of 1×Target Retrieval Solution is 6.1±0.2. 1×Target Retrieval Solution pH below 5.9 may give erroneous results. In alternative embodiments, pH of 1× Target Retrieval Solution after preparation is not adjusted.

In alternative embodiments, reagents are not equilibrated to room temperature before loading into the instrument. However, they can be loaded into the instrument before starting the staining procedure, which allows sufficient time for equilibration.

### Exemplary Staining Procedure

In alternative embodiments, the automated staining procedure for antibodies against Ki-67 on Dako OMNIS™ includes deparaffinization of tissue sections, target retrieval, and staining. In alternative embodiments, the slides are unloaded in the wet unloading station. All protocol steps can be pre-programmed into the Dako OMNIS™ software.

### Exemplary Pre-Staining Procedure:

1. Choose the Ki-67 IHC protocol or Ki-67 IHC NCR protocol to be applied for each slide from the Dako Link OMNIS™ Workstation software.
2. Ensure the Dako Link OMNIS™ Workstation software is configured to print slide labels with the protocol name displayed.
3. Print slide labels and attach them to the glass slides.
4. Place the slides in the Slide Rack. A Slide Rack can hold from one to five slides.
5. Ensure that the bulk bottles with fluids are onboard and registered by the Dako OMNIS™ instrument. Bulk bottle fluids:
   a. CLEARIFY CLEARING AGENT™ (Dako OMNIS™) (Code GC810)
   b. EnVision FLEX Target Retrieval Solution Low pH, (Dako OMNIS™) (Code GV805), diluted to 1×working concentration with distilled or de-ionized water
   c. Wash buffer (Dako OMNIS™) (Code GC807) diluted to 1×working concentration with distilled or de-ionized water
6. Ensure that all flip top vial caps are open and locked in place before loading all required reagents in the Reagent Storage Module:
a. Monoclonal Mouse Anti-Ki-67, Clone MIB-1, Code [GE020]
   b. Negative Control Reagent, Code [GE020]
   c. EnVision FLEX Peroxidase-Blocking Reagent (Dako OMNIS™), Code GV800 or GV823
   d. EnVision FLEX Visualization Reagent (Dako OMNIS™), Code GV800 or GV823
   e. EnVision FLEX Substrate Buffer (Dako OMNIS™), Code GV800 or GV823
   f. EnVision FLEX DAB+ Chromogen (Dako OMNIS™), Code GV800 or GV823
   g. Optional: Hematoxylin (Dako OMNIS™) Code GC808 or equivalent
   h. Sulfuric Acid, 0.3 M, (Dako OMNIS™) Code GC203
7. Load the Slide Rack onto Dako OMNIS™.
8. Follow the instructions on the Touch Screen and tap “Done” to initiate the staining procedure.
9. Ensure the slide unloading station is filled with distilled or de-ionized water to prevent slides from drying.

### Exemplary Counterstain Protocol

In alternative embodiments, slides are counterstained with Dako Hematoxylin (Code GC808). In alternative embodiments, the antibodies against Ki-67 and Ki-67 IHC Negative Control Reagent protocols on Dako OMNIS™ include a counterstaining step that is pre-programmed for 3 minutes with Hematoxylin (Dako OMNS™) (Code GC808). Slides are ready for mounting when removed from the Dako OMNIS™ unloading station.

### Exemplary Mounting Protocol

In alternative embodiments, after staining onboard Dako OMNIS™, the sections are dehydrated, cleared, and mounted using non-aqueous, permanent mounting methods.

### Exemplary Stained Slide Storage Protocol

Some fading of stained slides may occur, depending on several factors including, but not limited to, counterstaining, mounting materials and methods, and slide storage conditions. In alternative embodiments, to minimize fading, stained slides can be stored in the dark at room temperature (between about 20-25° C.).

### Exemplary System-Level Controls Protocol

In alternative embodiments, positive and negative control tissues (lab-supplied) are run for each staining procedure. In alternative embodiments, these quality controls are intended to ensure the validity of the staining procedure, including reagents, tissue processing and instrument performance. It is recommended that control tissues be stained on the same slide as the patient tissue. In alternative embodiments, the positive control is a tissue with positive biomarker expression. In alternative embodiments, the negative control is a tissue or tissue element with no biomarker expression. Control tissues should be fixed in the same way as the patient tissue. If controls are not fixed in the same way as the patient tissue, the control may only be used as a staining control for reagents and instrument performance.

### Exemplary Assay Verification Protocol

In alternative embodiments, prior to initial use of a staining system in a diagnostic procedure, or whenever there is a change in assay parameters, the user can verify the assay's performance by testing it on a series of lab-supplied tissues with known IHC performance characteristics representing known positive and negative tissues.

### Exemplary Negative Control Reagent Protocol

In alternative embodiments, a Negative Control Reagent (NCR) is used in place of the primary antibody with a section of each patient tissue to evaluate non-specific staining and allow better interpretation of specific staining at the antigen site.

### Exemplary Staining and Scoring Interpretation

In alternative embodiments, a hematoxylin and eosin (H&E) stained section adjacent to the IHC sample is used for the evaluation of an acceptable sample. In alternative embodiments, all viable invasive tumor cells on the entire slide are evaluated and included in the Ki-67 scoring assessment. In alternative embodiments, Ki-67 IHC scoring methods described herein and the H&E staining are performed on serial sections from the same paraffin block of the specimen to confirm:
1. The histological diagnosis of invasive breast cancer.
2. The specimen contains a minimum of 200 viable invasive tumor cells to determine the percentage of positive cells.
3. The specimen has been properly fixed and prepared for IHC analysis. Only well-preserved and well-stained areas of the specimen should be used to make the determination of the percentage of positive tumor cells.
4. Location of invasive cancer. Only the invasive cancer component should be scored. Carcinoma in situ should not be scored.

In alternative embodiments, slide evaluation is performed by a pathologist using a bright field microscope. In alternative embodiments, for evaluation of the immunohistochemical staining and scoring, an objective of 10-40× magnification is appropriate. In alternative embodiments, convincing nuclear staining of tumor cells with 1+ intensity or higher is included in the scoring. In alternative embodiments, the lower limit of 1+ positivity is evaluated using a high power (for example, 40×) objective and is defined by the following rules:
Signal must be unequivocally brown
The staining must correspond to a nucleus
The staining must cover the whole chromatin distribution within the nucleus
The staining must correspond to a viable (non-apoptotic, non-necrotic) cell Tumor areas and artifacts which should not be scored include:
Necrotic areas
In situ carcinoma areas
Edge effects
Fixation and processing artifacts

In alternative embodiments, Ki-67 protein expression is determined by assessing the percentage of viable tumor cells showing convincing nuclear staining at intensities 1+ and higher.

In alternative embodiments, For each staining procedure, slides should be examined in the order presented in Table 1 to determine the validity of the staining procedure and enable assessment of patient tissue staining.

### Exemplary Tissue Evaluation Protocol

The following Table 1 provides and exemplary order of tissue evaluation for interpretation of the exemplary Ki-67 IHC scores (%) as provided herein.

TABLE 1
Exemplary Recommended order of tissue evaluation

| Specimens | Rationale | Requirements |
|---|---|---|
| 1. Patient | A hematoxylin | The H&E and exemplary Ki-67 IHC |
| tissue | and eosin | antibody stain should be performed |
| stained | (H&E) stain of | on serial sections from the same |
| with H&E | the patient | paraffin block of the specimen. |
| | tissue is | Tissue specimens should be |
| | evaluated first | undamaged, well preserved, and |
| | to assess tissue | should confirm tumor indication. |
| | histology and | |
| | preservation | |
| | quality. | |
| 2. Positive | The positive | Controls should be biopsy/surgical |
| control | control tissue | specimens, fixed, processed and |
| tissue | stained with Ki- | embedded as soon as possible in the |
| stained | 67 Primary | same manner as the patient tissue(s). |
| with | Antibody should | Use undamaged specimens for |
| exemplary | be examined | interpretation of staining results as |
| Ki-67 IHC | next. Known | necrotic or degenerated cells often |
| primary | positive control | stain non-specifically. |
| antibody | tissue should | Controls should be normal tonsil or |
| (Lab- | only be utilized | biopsy/surgical specimens of the same |
| supplied) | for monitoring | tumor indication as the patient |
| | the correct | specimen, fixed, processed and |
| | performance of | embedded as soon as possible in the |
| | processed | same manner as the patient tissue(s). |
| | tissues and test | Use undamaged specimens for |
| | reagents, NOT | interpretation of staining results as |
| | as an aid in | necrotic or degenerated cells often stain |
| | formulating a | non-specifically |
| | specific | The tissue selected for use as the |
| | diagnosis of | positive tissue controls should include |
| | patient tissue. | moderate positive staining when |
| | | stained with Ki-67 to aid in detection of |
| | | subtle changes in assay sensitivity. |
| | | Positive control tissue should be |
| | | included in each staining procedure. |
| | | On-slide tissue controls are |
| | | recommended. |
| | | Tissue sections stained with Ki-67 |
| | | Primary Antibody: |
| | | 1. Tonsil: Tonsil stained with |
| | | antibodies against Ki-67 |
| | | should demonstrate moderate |
| | | to strong brown nuclear |
| | | expression in the majority of |
| | | germinal center B cells, The |
| | | parabasal layer of squamous |
| | | epithelium should show a |
| | | strong nuclear pattern. Cells in |
| | | the intermediate layer of |
| | | squamous epithelium should |
| | | demonstrate a low to moderate |
| | | nuclear expression. The |
| | | superficial layer and the |
| | | majority of cells in the basal |
| | | squamous epithelial layer |
| | | should be negative. |
| | | 2. Breast carcinoma: Presence of |
| | | brown nuclear staining should |
| | | be observed in tumor cells. |
| | | Cells labeled by the antibody display a |
| | | nuclear staining pattern except in |
| | | mitotic cells, where the chromosomes |
| | | and the cytoplasm are labeled. Non- |
| | | specific staining must be less than (<) |
| | | 1+. |
| | | If the positive control tissues fail to |
| | | demonstrate appropriate positive |
| | | staining, then results with the patient |
| | | tissue should be considered invalid. |
| 3.Optional: | Negative Control | Tissue sections stained with Negative |
| Positive | Reagent may be | Control Reagent: No nuclear staining. |
| control | used to stain the | Non-specific staining should be less |
| tissue | positive control | than (<) 1+. |
| stained | tissue specimen if | |
| with | needed for | |
| Negative | troubleshooting | |
| Control | purposes. | |
| Reagent | | |
| (NCR) | | |
| (Lab- | | |
| supplied) | | |
| 4. Negative | The negative | Controls should be biopsy/surgical |
| control | control tissue | specimens, fixed, processed and |
| tissue | stained with Ki- | embedded as soon as possible in the |
| stained with | 67 primary | same manner as the patient tissue(s). |
| antibodies | antibody should | Use undamaged specimens for |
| against Ki- | be examined next | interpretation of staining results as |
| 67 | to verify the | necrotic or degenerated cells often |
| (Lab- | labeling | stain non-specifically. |
| supplied) | specificity of the | Negative control tissue should be |
| | target antigen by | included in each staining procedure. |
| | the primary | On-slide tissue controls are |
| | antibody. | recommended. |
| | Note: When using | Negative control tissue may also be |
| | tonsil as a | normal tonsil. |
| | positive control | Tonsil sections stained with Ki-67 |
| | tissue, negative | Primary Antibody: |
| | control elements | The superficial layer and the majority |
| | within the | of cells in the basal squamous |
| | specimen may | epithelial layer should be negative. |
| | serve as the | This internal negative control element |
| | negative control | eliminates the need for a separate |
| | tissue. The use of | negative control tissue. Non-specific |
| | internal negative | staining must be less than (<) 1+ |
| | control elements | If the negative control tissues fail to |
| | in alternate | demonstrate appropriate staining, |
| | positive control | results with the patient tissue should |
| | tissue types must | be considered invalid. |
| | be verified by the | |
| | user. | |
| 5. | Negative Control | Tissue sections stained with Negative |
| Optional: | Reagent may be | Control Reagent: No nuclear staining. |
| Negative | used to stain the | Non-specific staining should be less |
| control | negative control | than (<) 1+. |
| tissue | tissue specimen if | |
| stained | needed for | |
| with | troubleshooting | |
| Negative | purposes. | |
| Control | | |
| Reagent | | |
| (NCR) | | |
| (Lab- | | |
| supplied) | | |
| 6.Optional: | Examine patient | Tissue stained with NCR should exhibit |
| Patient | tissue stained | no nuclear/membrane/cytoplasmic |
| tissue | with the Negative | staining in tumor cells. Non-specific |
| stained | Control Reagent. | staining should be less than (<) 1+. |
| using the | Negative Control | average staining across the specimen. |
| Negative | Reagent is used in | If weak staining is present in tumor |
| Control | place of the | nuclei, it should be used as a baseline |
| Reagent | primary antibody | to evaluate the Ki-67 antibody slide. |
| (NCR) | and aids in | Staining at the same intensity or lower |
| (Subject to | interpretation of | that may occur in the Ki-67 antibody |
| change | specific staining | slide should be disregarded upon |
| pending | at the antigen site. | interpretation. |
| FDA input | | If patient tissue stained with NCR |
| to final | | fails to demonstrate appropriate |
| version of | | staining, the corresponding patient |
| IVD IFU) | | tissue stained with primary antibody |
| | | is considered non-evaluable and the |
| | | patient tissue must be retested. |
| 7. Patient | Examine the | All viable invasive tumor cells should |
| tissue | patient tissue | be evaluated for Ki-67 and included |
| stained | stained with the | in the Ki-67 scoring assessment. |
| using | Ki-67antibody | Positive staining intensity should be |
| antibodies | last to assess Ki- | assessed within the context of any |
| against Ki- | 67 protein status. | non-specific staining observed in the |
| 6 | Refer to specific | staining procedure. NCR is |
| | information | recommended for this assessment if |
| | regarding | non-specific staining is observed. |
| | antibodies against | As with any immunohistochemical |
| | Ki-67 | test, a result showing no staining |
| | immunoreactivity. | means that the antigen was not |
| | | detected, not necessarily that the |
| | | antigen was absent in the cells/tissue |
| | | assayed. |

### Exemplary Performance Evaluation

### Non-Clinical Performance Evaluation: Normal and Neoplastic Tissues:

Normal tissues: The following Table 2 summarizes monoclonal mouse anti-Ki-67, Clone MIB-1 immunoreactivity on the recommended panel of normal tissues. Nuclear staining was observed in a subset of tissues. All tissues were formalin-fixed and paraffin-embedded and stained with antibodies against Ki-67 HC (on the Dako OMNIS™). There were no unexpected results observed in cell types or tissue types tested. The observed staining was consistent with the reported literature for Ki-67 IHC expression in normal tissues.

TABLE 2

| Tissue Type | | |
|---|---|---|
| (# tested) | Positive Cell Staining: Tissue Elements | Non-specific Staining |
| Adrenal (3) | 2/3 Scattered adrenal cortex cells | 0/3 |
| Bone marrow (3) | 2/3 Marrow cells | 0/3 |
| Breast (3) | 2/3 subset ductal epithelial cells | 0/3 |
| | 2/3 rare myoepithelial cells | |
| Cerebellum (3) | 0/3 | 0/3 |
| Cerebrum (3) | 1/3 Rare oligodendrocytes | 0/3 |
| Cervix (3) | 2/3 Rare epithelial cells | 0/3 |
| | 1/3 parabasal squamous cells | |
| Colon (3) | 3/3 Crypt epithelial cells | 0/3 |
| Esophagus (3) | 3/3 Parabasal squamous cells | 0/3 |
| Kidney (3) | 3/3 Rare tubule cells | 0/3 |
| Liver (3) | 3/3 Rare hepatocytes | 0/3 |
| Lung (3) | 3/3 Rare type 1 alveolar cells | 1/3 |
| | 1/3 Pulmonary macrophages | |
| Mesothelial cells (3) | 1/3 Rare mesothelial cells | 0/3 |
| Muscle, cardiac (3) | 1/3 Few cardiac myocytes | 0/3 |
| Muscle, skeletal (3) | 0/3 | 0/3 |
| Nerve, peripheral (3) | 0/3 | 0/3 |
| Ovary (3) | 2/3 Follicle cyst lining cells | 0/3 |
| | 1/3 Rare ovarian stromal cells | |
| Pancreas (3) | 3/3 Rare acinar cells | 0/3 |
| Parathyroid (3) | 3/3 Rare endocrine cells/Chief cells | 0/3 |
| Pituitary (3) | 1/3 Lymphocytes | 0/3 |
| | 2/3 Pituicytes | |
| Prostate (3) | 3/3 Rare epithelial cells and stromal cells | 0/2 |
| Salivary gland (3) | 1/3 Rare acinar epithelial cells | 0/3 |
| Skin (3) | 3/3 Suprabasal squamous cells | 0/3 |
| Small intestine (3) | 2/3 Crypt epithelium | 0/3 |
| Spleen (3) | 3/3 Few red pulp cells | 0/3 |
| Stomach (3) | 3/3 Gastric epithelial cells | 3/3 background in smooth muscle |
| Testis (3) | 3/3 Spermatogonia | 0/3 |
| Thymus (3) | 3/3 Thymic cortex | 0/3 |
| Thyroid (3) | 0/3 | 0/3 |
| Tonsil (3) | 3/3 Squamous parabasal cells | 0/3 |
| | 3/3 Germinal centers | |
| | 3/3 interfollicular cells | |
| Urinary bladder (3) | 3/3 Rare urothelial cells | 0/3 |
| Uterus (3) | 3/3 Endometrial epithelium and stroma | 0/3 |

Neoplastic tissues: The following Table 3 summarizes Monoclonal Mouse Anti-Ki-67, Clone MIB-1 immunoreactivity on a panel of neoplastic tissues. Nuclear staining was observed in the majority of tumor types evaluated. All tissues were formalin-fixed and paraffin-embedded and stained with antibodies against Ki-67 on the Dako OMNIS™. There were no unexpected results observed in the tumor specimens tested.

TABLE 3

| | Ki-67 |
|---|---|
| Tumor Type | positive/total |
| Colon mucinous adenocarcinoma | 0/1 |
| Gastric adenocarcinoma | 1/1 |
| Gastric adenocarcinoma metastasis | 1/1 |
| Colon adenocarcinoma in liver | 0/1 |
| Cholangiocarcinoma | 1/1 |
| Gastrointestinal stromal tumor (GIST) | 1/1 |
| Pancreatic adenocarcinoma | 1/1 |
| Squamous carcinoma of the ear | 1/1 |
| Kidney transitional cell carcinoma | 1/1 |
| Bladder transitional cell carcinoma | 2/2 |
| Ovarian dysgerminoma | 1/1 |
| Testicular yolk sac tumor | 1/1 |
| Testicular embryonal carcinoma | 1/1 |
| Renal cell carcinoma | 0/2 |
| Papillary renal cell carcinoma | 0/1 |
| Breast carcinoma, lobular | 0/2 |
| Breast carcinoma | 2/4 |
| Breast carcinoma, metastasis in lymph node | 1/1 |
| Thymoma | 1/1 |
| Splenic lymphoma | 1/1 |
| Lymphoma of cecum | 1/1 |
| Melanoma | 3/3 |
| Glial tumor | 1/1 |
| Meningioma | 1/1 |
| Hepatoma | 1/1 |
| Thyroid follicular adenoma | 1/1 |
| Ganglioneuroma | 0/1 |
| Thyroid papillary carcinoma | 0/1 |
| Prostate adenocarcinoma (metastatic) | 1/1 |
| Prostate adenocarcinoma | 0/1 |
| Lung bronchoalveolar carcinoma | 1/1 |
| Lung squamous carcinoma | 1/1 |
| Lung adenocarcinoma | 1/2 |
| Lung carcinoma | 1/1 |
| Fibrous tumor of pleura | 0/1 |
| Papillary serous carcinoma | 1/1 |
| Endometrial carcinoma | 0/1 |
| Ovarian mucinous adenocarcinoma | 0/1 |
| Well differentiated serous carcinoma | 0/1 |
| Metastatic ovarian carcinoma | 1/1 |
| Endometrial stromal sarcoma | 1/1 |
| Ovary granulosa cell tumor | 1/1 |
| Squamous carcinoma, cervix | 1/1 |
| Merkel cell tumor | 1/1 |
| Islet cell tumor of Pancreas | 1/1 |
| Pancreatic glucagonoma | 1/1 |
| Schwanomma | 1/1 |
| Thymic carcinoid Tumor | 2/2 |
| Pheochromocytoma | 1/1 |
| Paraganglioma | 0/1 |
| Thyroid medullary carcinoma | 1/1 |
| Pleomorphic rhabdomyosarcoma | 1/1 |
| Spindle cell rhabdomyosarcoma | 0/1 |
| Ewings sarcoma | 0/1 |
| Primitive neuroendocrine tumor in scrotum | 0/1 |
| Leiomyosarcoma | 1/1 |
| Round cell liposarcoma | 1/1 |
| Synovial sarcoma | 0/1 |
| Malignant fibrous Histocytoma | 1/1 |
| Extraskeletal. myxoid chondrosarcoma | 0/1 |
| Basal cell carcinoma, skin | 0/1 |

### Control Tissues:

Differences in processing and embedding in the user's laboratory may produce significant variability in results. It is recommended that control tissues be stained on the same slide as the patient tissue. See Table 1, above.

Include positive and negative in-house control tissue in each staining run. Controls should be normal tonsil or biopsy/surgical specimens of the same tumor indication as the patient specimen, fixed, processed, and embedded as soon as possible in the same manner as the patient tissue(s). When using tonsil as a positive control tissue, negative control elements within the tonsil specimen may serve as the negative control tissue.

Control tissues processed differently from the patient specimen validate reagent performance only and do not verify proper patient tissue preparation.

The tissue selected for use as the positive tissue controls should include weak-to-moderate positive staining when stained with Ki-67 IHC as provided herein to aid in detection of subtle changes in assay sensitivity.

Tonsil stained with Ki-67 IHC as provided herein should demonstrate moderate to strong brown nuclear expression in the majority of germinal center B cells. The parabasal layer of squamous epithelium should show a strong nuclear pattern. Cells in the intermediate layer of squamous epithelium should demonstrate a low-to-moderate nuclear expression. The superficial layer and the majority of cells in the basal squamous epithelial layer should be negative.

When using biopsy/surgical specimens as control tissue that are the same tumor indication as the patient's specimen, the presence of brown nuclear staining should be observed in tumor cells. The ideal positive control tissue provides a complete dynamic representation of weak-to-moderate staining of tumor cells. The ideal negative control tissue should demonstrate no staining on tumor cells.

### Tissue Processing

Formalin-fixed, paraffin-embedded tissues have been validated for use. Other tissue preparations (for example, cytology specimens, fine-needle aspirates or bone decalcifications) can also be used. In alternative embodiments: block specimens into a thickness of 3 mm or 4 mm, fix in formalin and dehydrate and clear in a series of alcohols and xylene, followed by infiltration with melted paraffin. In alternative embodiments, the paraffin temperature is set not to exceed 60° C. Feasibility studies on breast carcinoma tissue samples were performed with fixation in 10% neutral buffered formalin for 6-72 hours.

In alternative embodiments, cut tissue specimens into sections of 4 μm to 5 μm. In alternative embodiments, after sectioning, tissues are mounted on Dako FLEX™ IHC Microscope Slides or SUPERFROST PLUS™ slides, and then placed in an oven and dried at 58° C.±2° C. for 1 hour. In alternative embodiments, all tissues, specimens and controls are mounted within the validated area of the slide. In alternative embodiments, to preserve antigenicity, store tissue sections in the dark at 2° C. to 8° C. (preferred) or at room temperature up to 25° C. and stain within 2 months of sectioning.

### Staining:

Reagent Storage: Store all components according to the IFU when not in use.

Reagent Preparation: Reagents do not need to be equilibrated to room temperature before loading into the instrument. However, they should be loaded into the instrument before starting the staining procedure, which allows sufficient time for equilibration.

In alternative embodiments, EnVision FLEX Target Retrieval Solution, Low pH and Wash Buffer: EnVision FLEX Target Retrieval Solution, Low pH (50×) (GV805) and Wash Buffer (20×) (GC807) are diluted to 1×concentration according to their Instructions for Use.

Deparaffinization, Rehydration, Target Retrieval, Staining, and Counterstaining: Select the Ki-67 IHC protocol or Ki-67 IHC NCR protocol for the slides to be stained; Place the Dako OMNIS™ staining rack with slides on the Dako OMNIS™ instrument; Load all required reagents onboard the Dako OMNIS™ as prompted by the instrument. Ensure that the appropriate quality and amount of water is added to the unloading rack to prevent specimen dehydration; The instrument performs the pretreatment, staining, and counterstaining procedures by applying the appropriate reagent, monitoring the incubation time, and rinsing slides between reagents.

Mounting: In alternative embodiments, non-aqueous permanent mounting media are used. To minimize fading, store slides in the dark at room temperature (20-25° C.).

### Product-Specific Limitations:

In one embodiment, a minimum of 200 viable invasive tumor cells are present in the Ki-67 stained slide for the specimen to be considered adequate for Ki-67 evaluation. If less than 200 viable invasive tumor cells are present, tissue from a deeper level of the block, or potentially another block, could have a sufficient number of viable tumor cells for Ki-67 IHC testing.

Positive and Negative In-house Control Tissue: Examine the positive in-house control tissue (tonsil or breast carcinoma) to determine that the tissues are correctly prepared and the reagents are functioning properly. In alternative embodiments, it is recommended that control tissues be stained on the same slide as the patient tissue. The ideal positive control tissue provides a complete dynamic representation of weak-to-moderate staining of cells. If staining of positive in-house control tissue is not satisfactory, all results with the patient specimen should be considered invalid.

When Using Tonsil as a Positive Control Tissue, Negative Control Elements within the Specimen May Serve as the Negative Control Tissue: Tonsil stained with Ki-67 IHC as provided herein should demonstrate moderate-to-strong nuclear expression in the majority of germinal center B cells. The parabasal layer of squamous epithelium should show a strong nuclear pattern. Cells in the intermediate layer of squamous epithelium should demonstrate low-to-moderate nuclear expression. The superficial layer and the majority of cells in the basal squamous epithelial layer should be negative. Cells labeled by the antibody display a nuclear staining pattern except in mitotic cells, where both the mitotic nuclei and the cytoplasm are labeled.

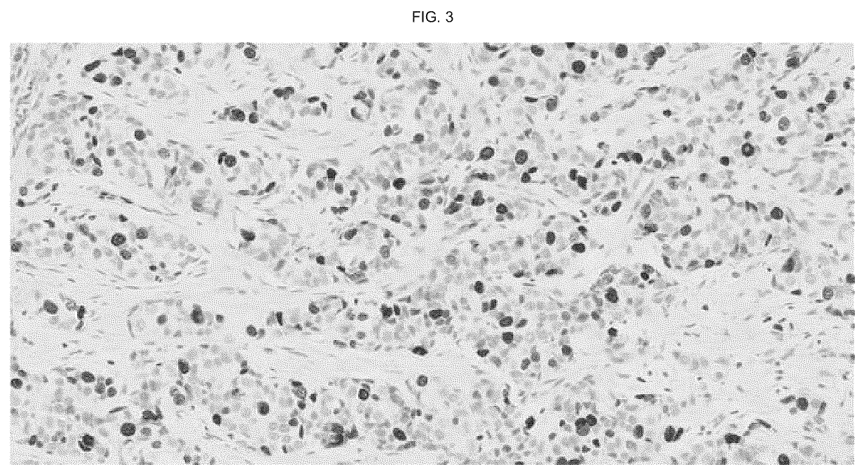

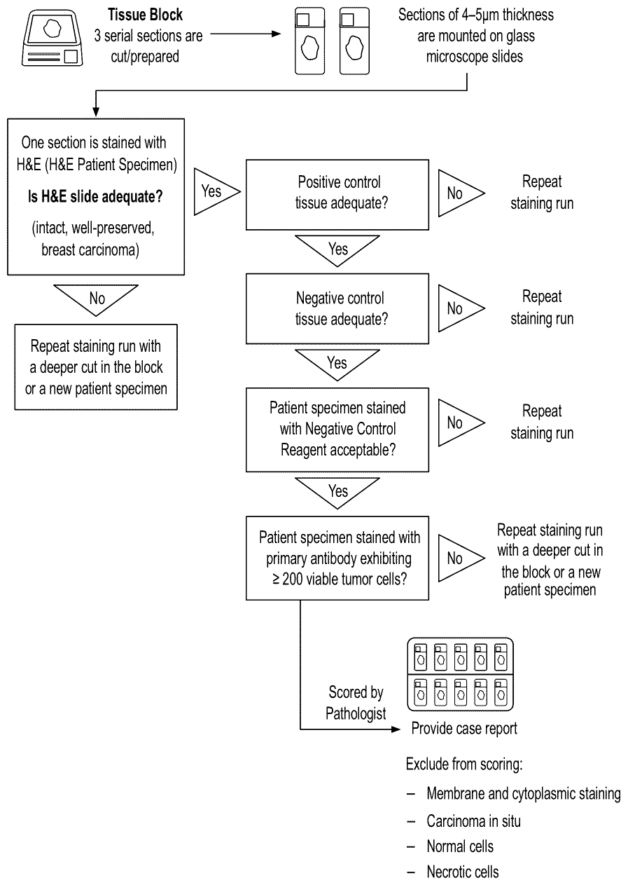

Using Breast Carcinoma as In-House Control Tissue: The ideal positive control tissue provides a complete dynamic representation of weak-to-moderate staining of cells. (FIG. 3). In alternative embodiments, control tissues are stained on the same slide as the patient tissue. In alternative embodiments, when using breast carcinoma as an on-slide control tissue, the positive control tissue are run on the same slide as patient specimen. The negative control may be run on a separate slide. If the staining of positive in-house control tissue is not satisfactory, all results with the patient specimen should be considered invalid. FIG. 3 illustrates positive in-house control tissue stained with exemplary Ki-67 IHC showing different intensities of nuclear Ki-67 expression by invasive breast carcinoma cells (20× magnification).

The ideal negative control tissue should demonstrate no staining of tumor cells. Examine the negative in-house control tissue to determine the expected staining. The variety of different cell types present in most breast carcinoma tissue sections offers internal negative control sites; this should be verified by the user.

If inappropriate staining occurs in the in-house control tissues, results with the patient specimen should be considered invalid.

Negative Control Reagent (NCR): In alternative embodiments, examine the slides stained with the NCR to identify non-specific background staining that may interfere with Ki-67 staining interpretation, making the specimen non-evaluable. Satisfactory performance is indicated by the absence of specific staining; examine the patient specimens stained with the NCR to determine if there is any non-specific staining that may interfere with interpreting the Ki-67 stained slide.

An exemplary protocol for evaluating staining is schematically illustrates in FIG. 4.

In alternative embodiments, Ki-67 expression in breast carcinoma stained with the exemplary Ki-67 THC is determined by using the following exemplary Ki-67 Score (%). In alternative embodiments, to determine the Ki-67 Score (%), the entire viable tumor area must be evaluated. Ki-67 Score (%) is the number of Ki-67 staining viable invasive tumor cells divided by the total number of viable invasive tumor cells in the entire specimen, multiplied by 100%.

$$Determining Ki - 67 Score ( \% ) Ki - 67 score ( \% ) = \frac{\# of Ki - 67 staining viable invasive tumor cells}{Total \# of staining and non - staining viable invasive tumor cells} \times 100$$

Any convincing nuclear staining greater than or equal to (≥) 1+ of viable invasive tumor cells is considered Ki-67 staining and should be included in the scoring. Nuclear staining (≥1+) of any other cell type, including in situ breast carcinoma, non-neoplastic breast epithelium or other non-neoplastic cells should be excluded from the Ki-67 Score (%) calculation.

### Exemplary Protocol for Determining Ki-67 Score (%):

At lower magnifications, examine all well-preserved tumor areas in the entire slide.

Evaluate overall areas of Ki-67 staining and non-staining tumor cells, keeping in mind that 1+ nuclear staining may be difficult to see at low magnifications

In alternative embodiments, a minimum of 200 viable invasive tumor cells are present in the Ki-67 stained slide for the specimen to be considered adequate for evaluation For specimens with less than 200 viable tumor cells, sections from a deeper level of the block or potentially another block could have a sufficient number of tumor cells for evaluation of Ki-67 expression
At higher magnification, evaluate Ki-67 expression and determine Ki-67 Score (%):
Estimate the total number of viable invasive tumor cells, both Ki-67 staining and non-staining (Ki-67 Score (%) denominator)
Estimate the number of Ki-67 staining viable invasive tumor cells (Ki-67 Score (%) numerator; see Tables 1 and 2 for additional Ki-67 Score (%) inclusion/exclusion criteria)

### Estimate Ki-67 Score (%)

TABLE 4
Ki-67 Score (%) Numerator Inclusion/Exclusion Criteria

| | Included in the | Excluded from the |
|---|---|---|
| Tissue Elements | Numerator | Numerator |
| Tumor Cells | Viable invasive | Non-staining tumor |
| | tumor cells with | cells |
| | convincing and | Tumor cells with |
| | complete nuclear | only cytoplasmic or |
| | staining (at any | membrane staining |
| | intensity 1+ and | Non-invasive |
| | higher) | neoplasia (including |
| | | carcinoma in situ) |
| | | Non-viable/necrotic |
| | | tumor cells |
| | | Apoptotic |
| | | nuclei/nuclear debris |
| | | Tumor cells in |
| | | poorly preserved |
| | | tissue areas (ie poor |
| | | fixation, processing |
| | | artifacts) |
| | | Inflammatory breast |
| | | cancer |
| Other Cells | Not included | Benign |
| | | epithelial cells |
| | | Other non- |
| | | neoplastic cells |

TABLE 5
Ki-67 Score (%) Denominator Inclusion/Exclusion Criteria

| | Included in the | Excluded from the |
|---|---|---|
| Tissue Elements | Denominator | Denominator |
| Tumor Cells | All viable invasive | Non-invasive |
| | tumor cells | neoplasia (including |
| | | carcinoma in situ) |
| | | Non-viable/necrotic |
| | | tumor cells |
| | | Apoptotic |
| | | nuclei/nuclear debris |
| | | Tumor cells in poorly |
| | | preserved tissue |
| | | areas (i.e., poor |
| | | fixation, processing |
| | | artifacts) |
| | | Inflammatory breast |
| | | cancer |
| Other Cells | Not included | Benign |
| | | epithelial cells |
| | | Other non- |
| | | neoplastic cells |

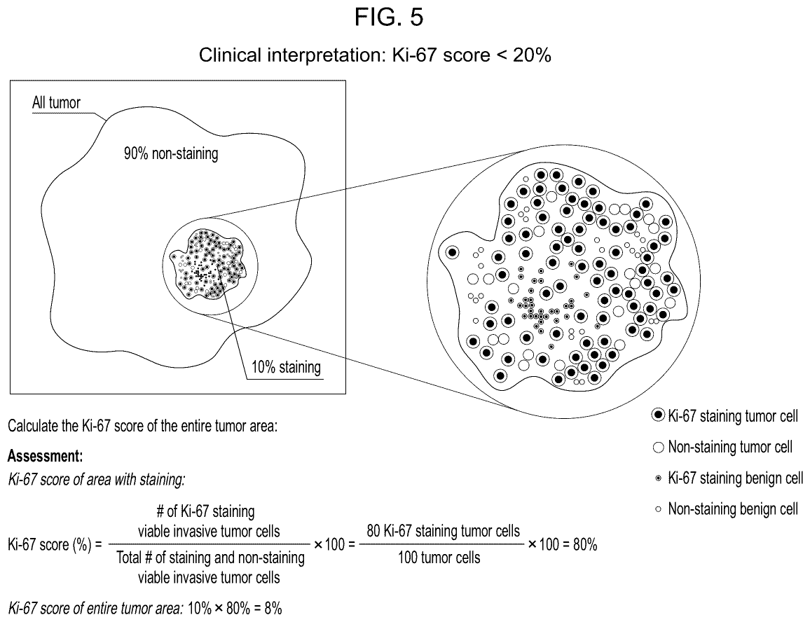

FIG. 5 illustrates an example of a determination of a Ki-67 score (%) based on a small Ki-67 staining area:

First the tumor area is evaluated for convincing staining as described in “Determining Ki-67 Score (%)”. Assessment: 10% of area shows staining, 90% of area shows no staining.

Second: Evaluate the area of staining to determine the number of Ki-67 staining invasive tumor cells. Assessment: There are approximately 100 viable tumor cells in the area of staining and about 80 Ki-67 staining cells (per the Ki-67 Score (%) numerator).

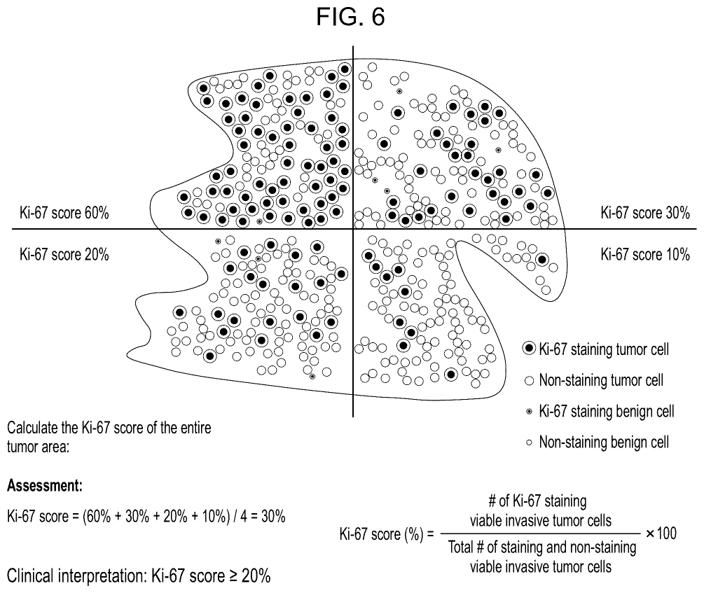

FIG. 6 illustrates determination of Ki-67 Score (%) Based on a Heterogeneous Ki-67 Staining Area:

First: Visually divide the tumor area into regions with equal numbers of tumor cells.

Second: Observe each region and determine the total number of viable tumor cells and Ki-67 staining tumor cells. Determine the Ki-67 Score (%) for each region.

Assessment: The four sections have approximately 60, approximately 30, approximately 20, and approximately 10 Ki-67 staining tumor cells. Each section has a total of 100 tumor cells (including Ki-67 staining cells). The Ki-67 Score (%) for each section: approximately Ki-67 Score 60%, approximately Ki-67 Score 30%, approximately Ki-67 Score 20%, and Ki-67 Score 10%.

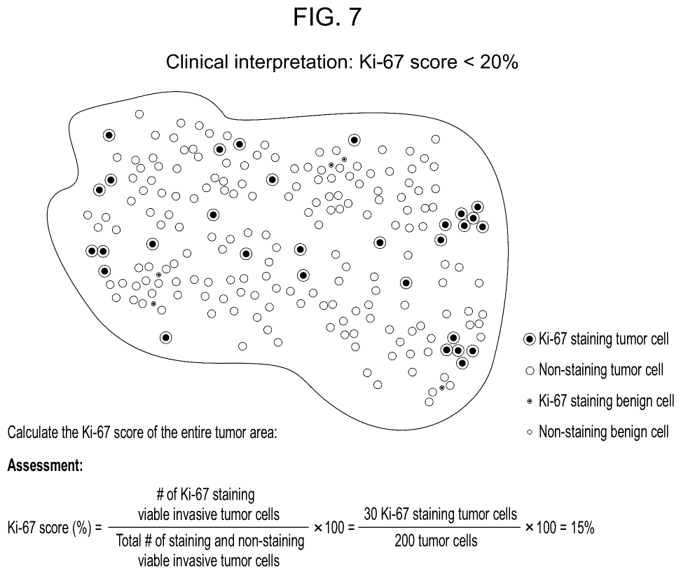

FIG. 7 illustrates determination of Ki-67 Score (%) for a Near Cut-off Specimen:

First: Evaluate the specimen for convincing staining as described in “Determining Ki-67 Score (%)”.

Second: Examine the specimen at a higher objective (20×) to confirm that there is no weak (1+) staining in areas that appeared devoid of staining at lower objectives. Evaluate all staining areas and estimate the total number of Ki-67 staining tumor cells.

Then re-evaluate the entire specimen (staining and non-staining areas) and estimate the total number of viable invasive tumor cells (Ki-67 staining and non-staining tumor cells). Determine the Ki-67 Score (%).

Assessment: Tumor specimen has perceptible and convincing staining. 30 Ki-67 staining invasive tumor cells. There are approximately 200 viable invasive tumor cells present in the entire specimen.

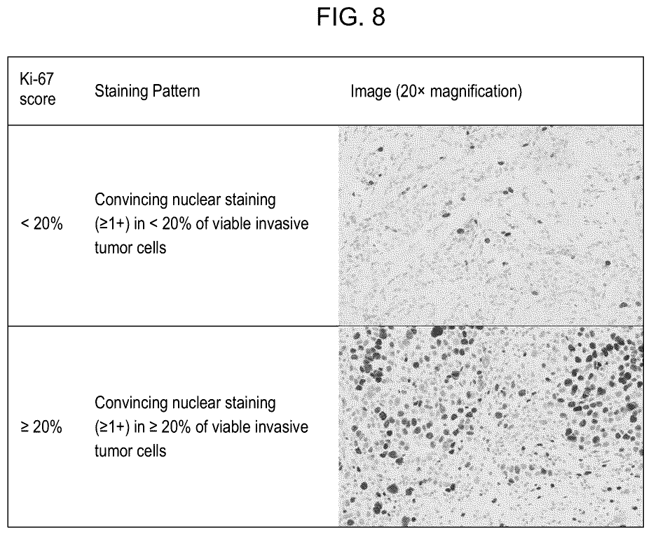

FIG. 8 provides examples illustrating how the Ki-67 Score (%) determines the percentage of viable invasive tumor cells with nuclear Ki-67 expression, with the Ki-67 Score (%) and corresponding Ki-67 expression levels.

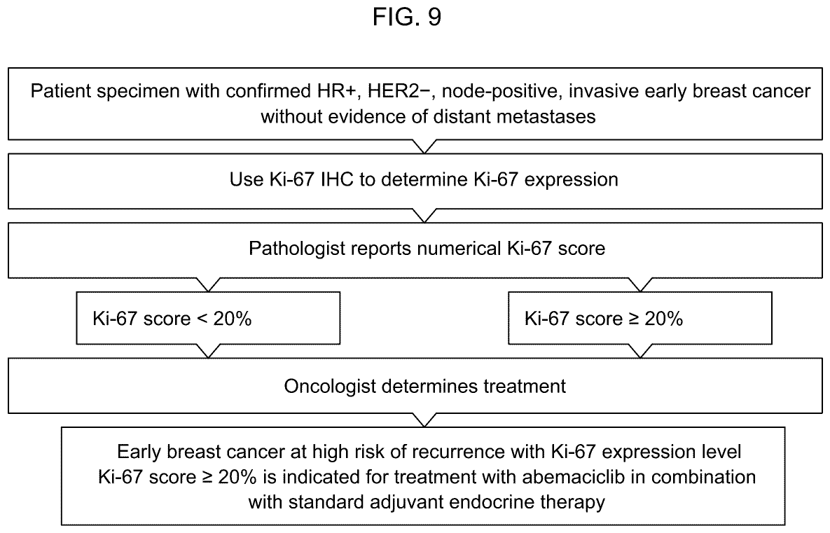

FIG. 9 schematically illustrates a flowchart that can be used to decide which patients are indicated for treatment with abemaciclib, palbociclib (optionally IBRANCE™ or PALBONIX™), or ribociclib (optionally KISQUALI™) or other cyclin dependent kinase inhibitors, such as CDK4 or CDK6 inhibitors based on their Ki-67 Score (%).

In summary: Ki-67 staining cells in invasive breast carcinoma are: Viable tumor cells with convincing nuclear staining (at intensity 1+ or higher) that corresponds to the chromatin distribution within the nucleus; and, Ki-67 expression status in breast carcinoma is determined by Ki-67 Score (%), which is the number of Ki-67 staining invasive tumor cells divided by the total number of viable invasive tumor cells in the entire specimen, multiplied by 100%.

### Ki-67 Staining Cells Included in Ki-67 Score (%):

Tumor cells exhibiting appropriate Ki-67 expression are defined as Ki-67 staining cells showing convincing nuclear staining at any intensity greater than or equal to (≥) 1+. All Ki-67 staining viable invasive tumor cells are included in the Ki-67 Score (%) numerator for determination of the Ki-67 Score (%) (see Tables 1 and 2 for additional Ki-67 Score (%) inclusion/exclusion criteria). All viable invasive tumor cells should be included in the denominator. Below are common staining characteristics of Ki-67 staining cells that should be included in the Ki-67 Score (%) numerator:

Nuclear staining of tumor cells at all intensities 1+ to 3+ should be included. Tumor cells exhibiting convincing nuclear staining are considered Ki-67 staining cells. Convincing nuclear staining is determined by the following parameters:
1. The signal is unequivocally brown.
2. The staining corresponds to a nucleus.
3. The staining covers the whole chromatin distribution within the nucleus.
4. The staining corresponds to viable (non-apoptotic, non-necrotic) cells.

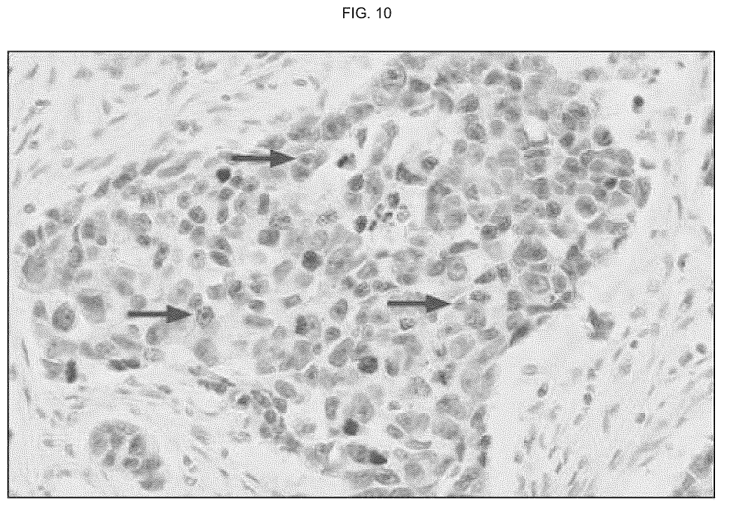

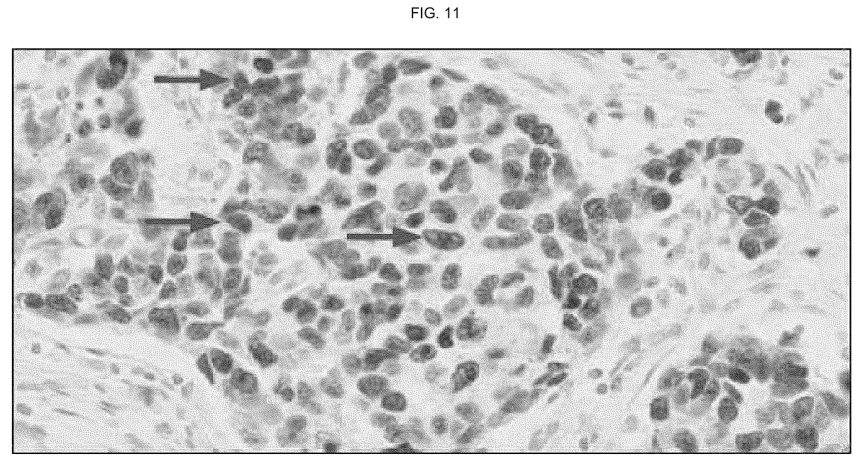

FIG. 10 illustrates an image of an invasive breast carcinoma specimen stained with an exemplary Ki-67 IHC exhibiting 1+ nuclear staining of tumor cells (arrows).

FIG. 11 illustrates an image of an invasive breast carcinoma specimen stained with an exemplary Ki-67 IHC exhibiting 2+ nuclear staining of tumor cells (arrows).

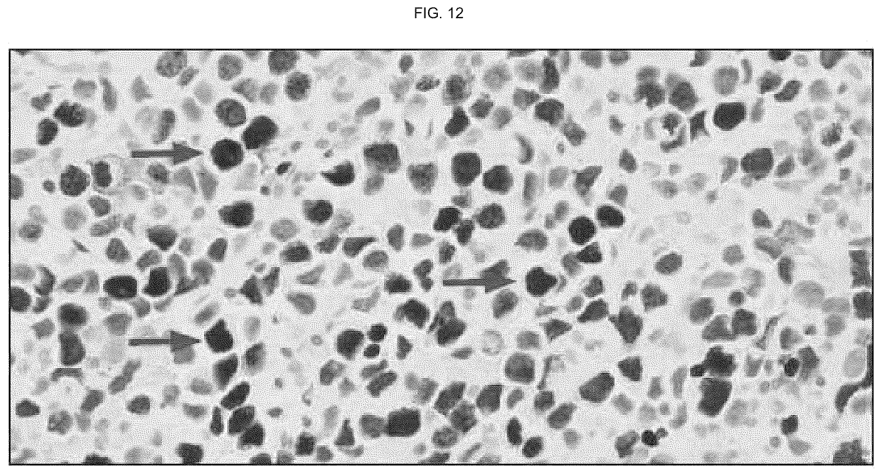

FIG. 12 illustrates an image of an invasive breast carcinoma specimen stained with an exemplary Ki-67 IHC exhibiting 3+ nuclear staining of tumor cells (arrows).

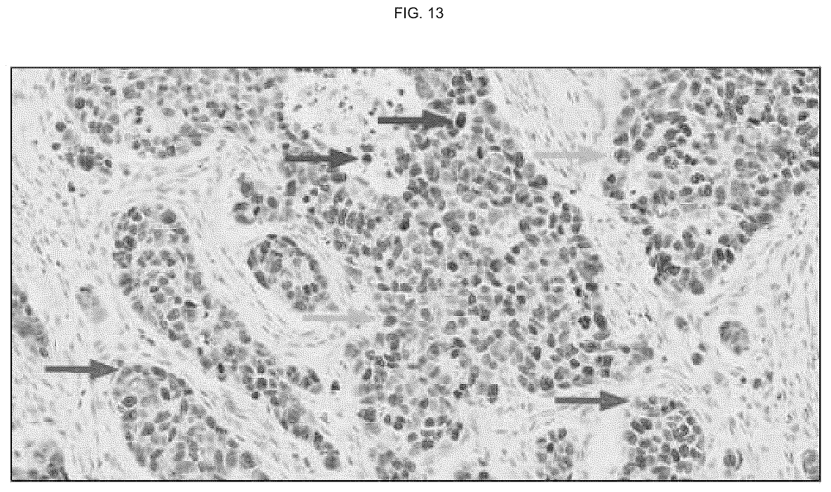

Convincing staining of tumor cells is often heterogeneous, with various staining intensities present in the same sample. FIG. 13 illustrates an image of an invasive breast carcinoma specimen stained with an exemplary Ki-67 IHC exhibiting 1+ to 3+ nuclear staining of tumor cells. Red arrows indicate 3+ staining intensities, yellow indicate 2+ staining intensities, and green indicate 1+ staining intensities (20×objective).

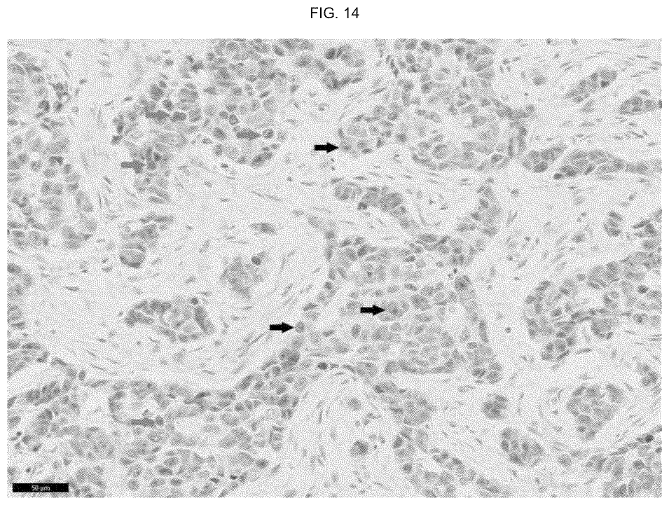

Cells that exhibit a “grey” coloring in the nucleus are excluded from Ki-67 scoring. If the nucleus is not unequivocally brown in color, then the cell is considered to not be exhibiting convincing nuclear staining. FIG. 14 illustrates an image of an invasive breast carcinoma specimen stained with antibodies against Ki-67 exhibiting both negative and weak positive staining. Negative cells show grey hematoxylin counterstaining and are indicated with black arrows, and weak 1+ staining indicated with green arrows (20× objective).

In summary: convincing nuclear staining of viable invasive tumor cells at any intensity should be included in the Ki-67 Score (%) numerator.

Tumor cells with membrane and/or cytoplasmic staining at any objective should not be included in the Ki-67 Score (%) numerator unless the nucleus is also clearly staining as defined in the previous sections:

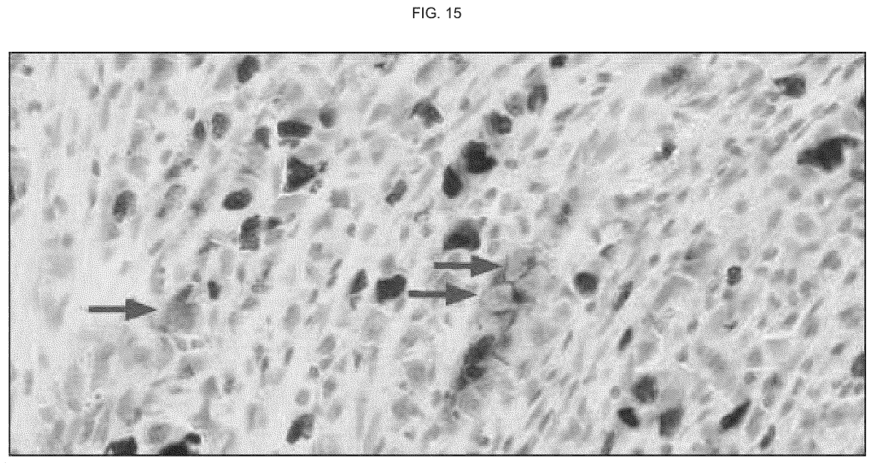

FIG. 15 illustrates an image of an invasive breast carcinoma specimen stained with an exemplary Ki-67 antibody exhibiting membrane staining with no distinct nuclear staining (arrows) (20× objective).

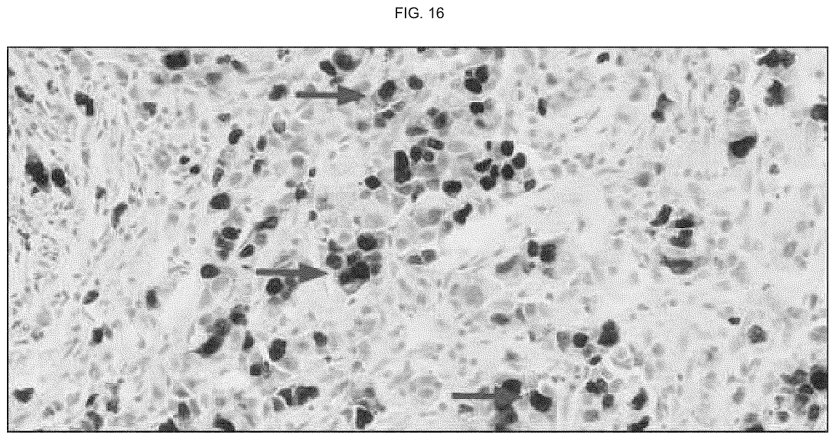

FIG. 16 illustrates an image of an invasive breast carcinoma specimen stained with an exemplary Ki-67 exhibiting cytoplasmic staining with distinct nuclear staining (arrows) (20× objective).

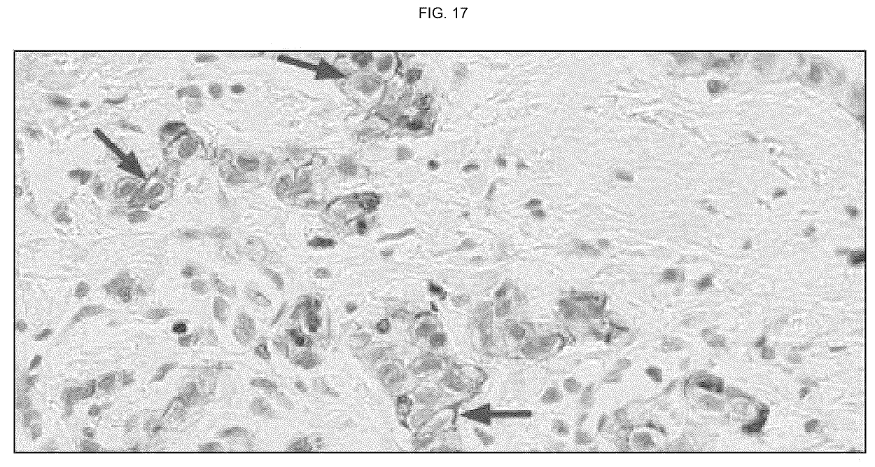

FIG. 17 illustrates an image of an invasive breast carcinoma specimen stained with antibodies against Ki-67 exhibiting membrane staining with no distinct nuclear staining (arrows) (20× objective).

In summary: Tumor cells exhibiting perceptible membrane and/or cytoplasmic staining without nuclear staining are not included in the Ki-67 Score (%) numerator. Tumor cells that do show distinct nuclear staining in addition to membrane and/or cytoplasmic staining should be included in the Ki-67 Score (%) numerator.

Uneven Chromatin Distribution: Occasionally tumor cells can exhibit an incomplete nuclear staining pattern due to uneven chromatin distribution or nuclear pseudo-inclusions, resulting in a “nuclear clearing” aspect. These nuclear features should also be observed on the corresponding H&E stain. Any convincing nuclear staining of tumor cells that covers the entirety of the chromatin distribution within the nucleus (≥1+ intensity) should be included in the Ki-67 Score (%) numerator, even if incomplete in appearance.

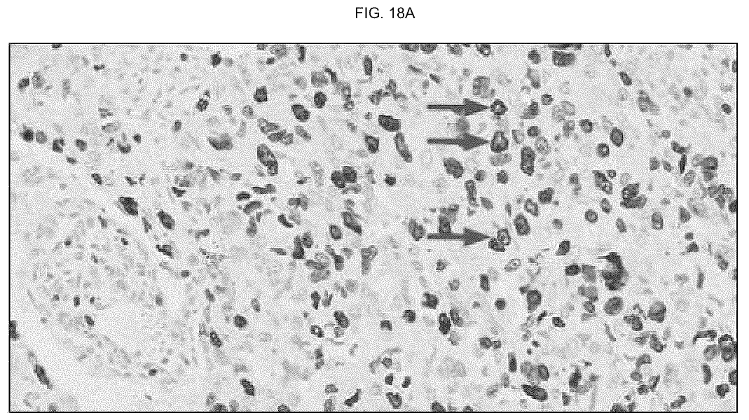

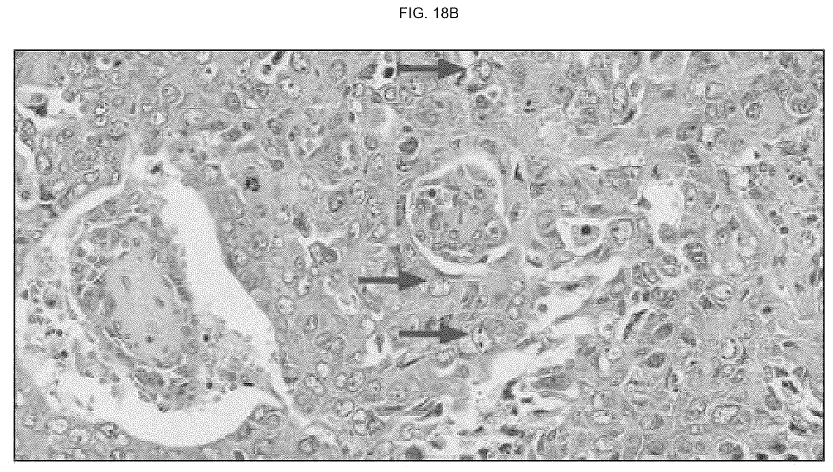

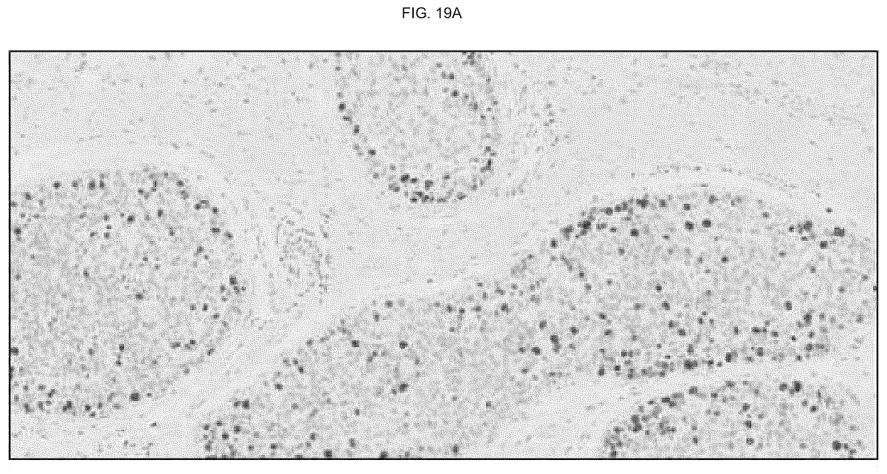

FIG. 18A-B illustrate images of an invasive breast carcinoma specimen stained with an exemplary Ki-67 IHC (FIG. 18A) and corresponding H&E stained slide (FIG. 18B) exhibiting incomplete nuclear staining pattern due to uneven chromatin distribution that results in a “nuclear clearing” aspect (arrows) (20×objective). Tumor cells must exhibit a convincing nuclear staining pattern corresponding to the chromatin distribution within the nucleus to be included in the Ki-67 Score (%) numerator.

Cells Excluded from Ki-67 Score (%)

Only viable invasive tumor cells exhibiting convincing Ki-67 nuclear staining should be included in the Ki-67 Score (%) numerator. In alternative embodiments, cells that can exhibit staining but should be excluded from the Ki-67 Score (%) estimation (Ki-67 Score (%) numerator and/or denominator) can include:

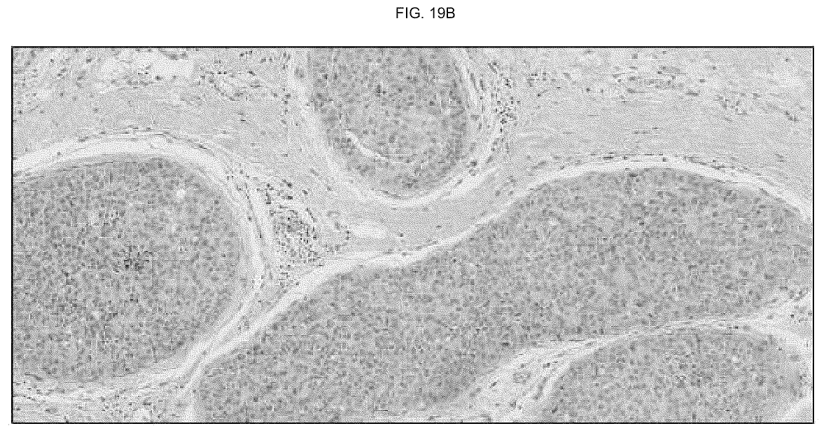

Carcinoma In Situ: Carcinoma in situ cells exhibiting Ki-67 nuclear staining should be excluded from the Ki-67 Score (%) estimation. FIG. 19A-B illustrate images of an invasive breast carcinoma specimen stained with and exemplary Ki-67 IHC (FIG. 19A) exhibiting staining of ductal carcinoma in situ (DCIS) and corresponding H&E (FIG. 19B) (10× objective).

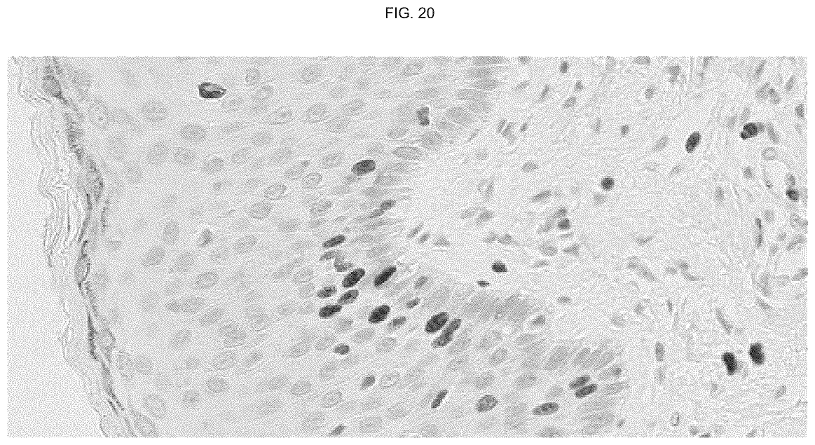

Benign epithelial cells can show Ki-67 nuclear staining. These cells should be excluded from Ki-67 Score (%) estimation. FIG. 20 illustrates an image of staining using an exemplary Ki-67 IHC in normal epidermal cells adjacent to invasive area of breast carcinoma specimen (20× objective).

Non-neoplastic breast epithelium such as normal ducts and lobules adjacent to breast carcinoma may show Ki-67 positive nuclear staining. These cells should not be included in the estimation of Ki-67 Score (%). FIG. 21 illustrates an image of a breast carcinoma specimen stained with an exemplary Ki-67 IHC, where Ki-67 staining in normal epithelial cells within ducts and lobules can be seen (arrows). The left half of the image above shows tumor cells that should be included in Ki-67 Score (%) calculation. The right side of the image shows normal breast epithelium that should excluded from the Ki-67 Score (%) estimation (10× objective).

Only viable tumor cells should be included in the Ki-67 Score (%) estimation. Non-viable, necrotic and apoptotic cells that may or may not be exhibiting Ki-67 staining should be excluded from both the numerator and denominator. FIG. 22A-B illustrate images of a necrotic area of breast carcinoma specimen stained with Ki-67 primary antibody (FIG. 22A) and corresponding H&E stained slide (FIG. 22B) (10×objective).

Lymphocytes often exhibit nuclear staining and should not be included in the Ki-67 Score (%) scoring algorithm. Nuclear staining of lymphocytes is often heterogeneous, with various staining intensities present. FIG. 23A illustrates an image of breast carcinoma specimen stained with an exemplary Ki-67 exhibiting staining of scattered positive lymphocytes within a lymphoid aggregate (arrows) (20×objective). FIG. 23B illustrates an image of breast carcinoma specimen stained with an exemplary Ki-67 IHC exhibiting staining of lymphocytes (arrows) admixed with positive tumor cells (20× objective).

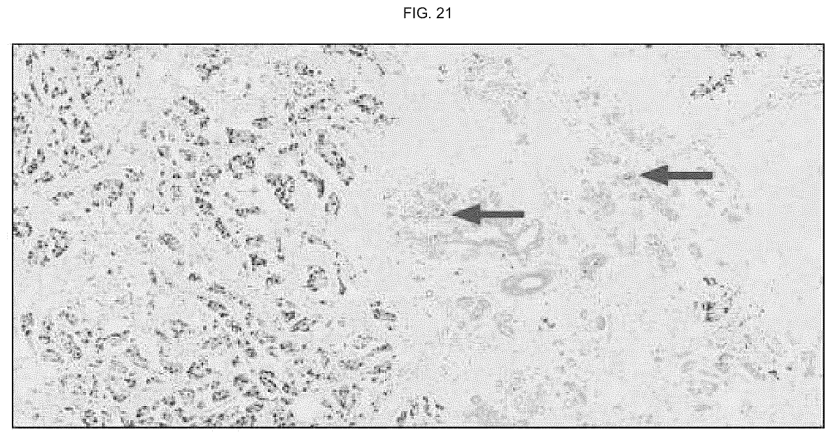

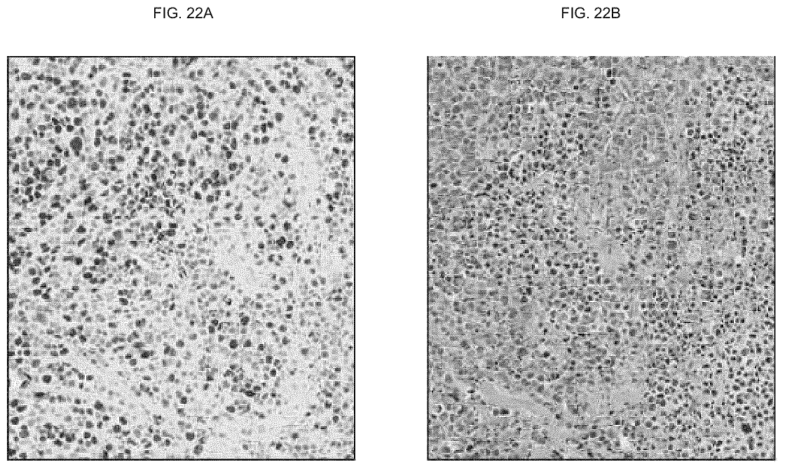

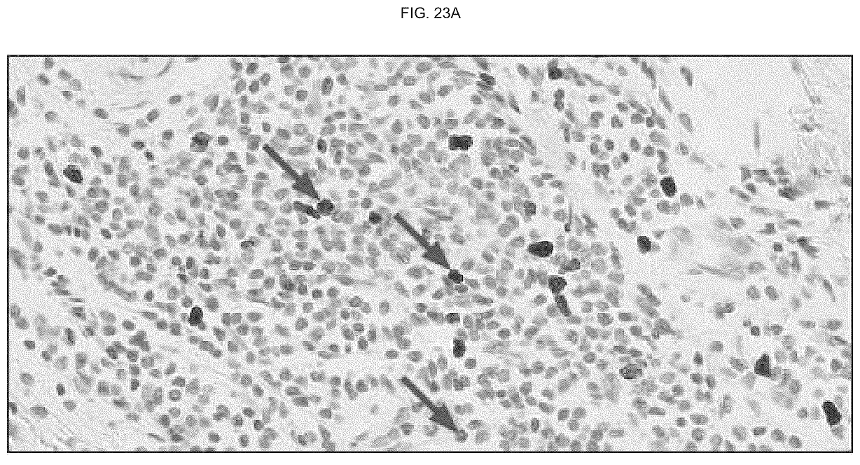

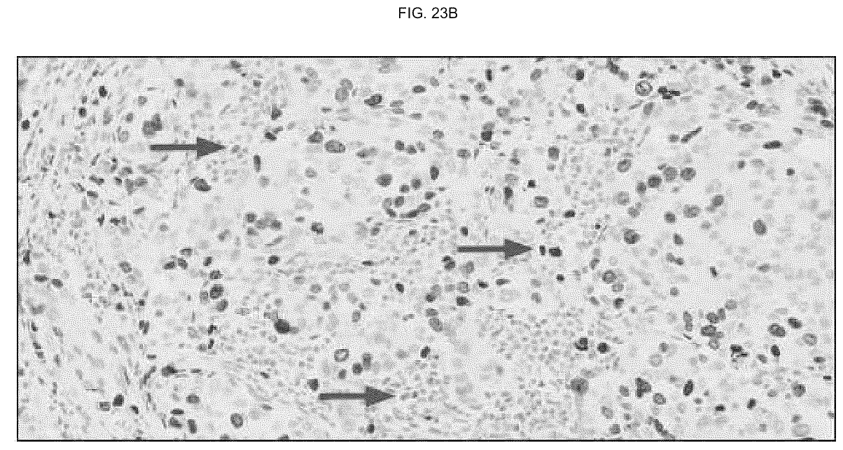

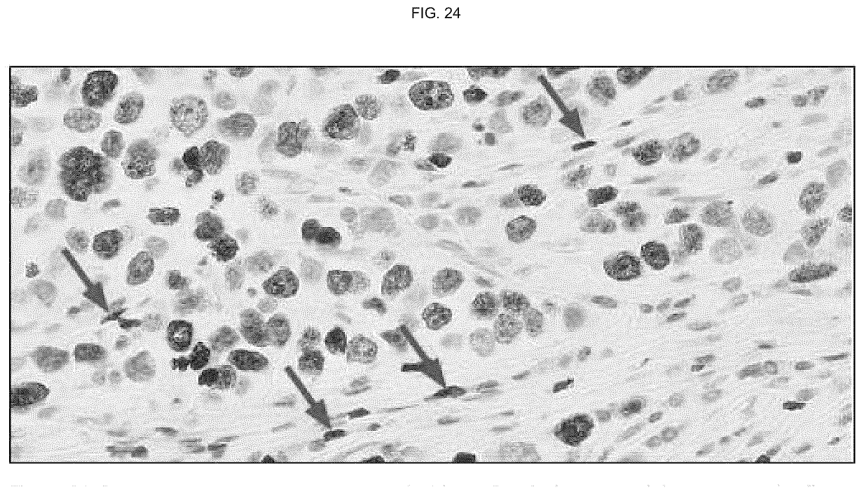

Stromal cells exhibiting Ki-67 nuclear staining should be excluded from the Ki-67 Score (%) estimation. FIG. 24 illustrates an image of a breast carcinoma specimen stained with an exemplary Ki-67 HC exhibiting stromal cell nuclear staining (arrows), as well as staining of tumor cells (20× objective).

### Pre-Analytical Artifacts:

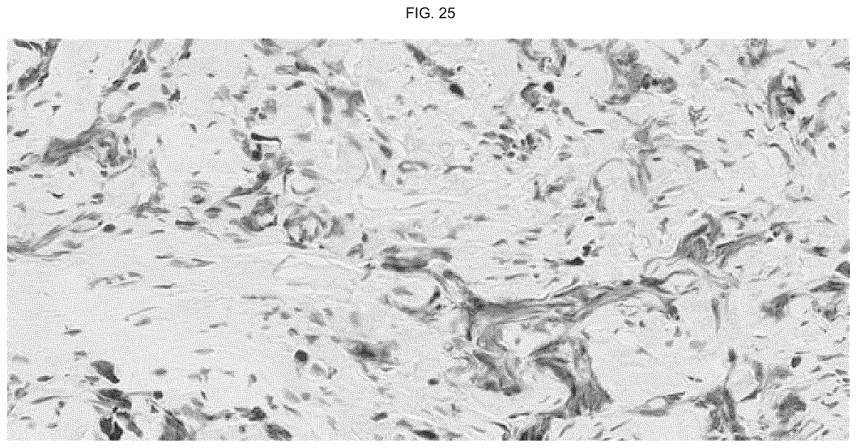

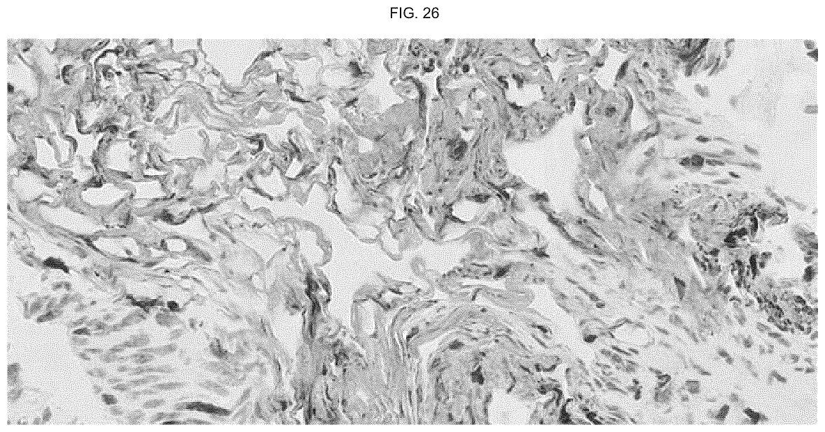

Areas of the examined section exhibiting distorted morphology secondary to pre-analytical artifacts such as poor fixation, crush and/or cautery artifact should be excluded from scoring. FIG. 25 illustrates an image of a breast carcinoma specimen stained with an exemplary Ki-67 IHC exhibiting crush artifact; these areas should be excluded from the score (20× objective). FIG. 26 illustrates an image of a breast carcinoma specimen stained with an exemplary Ki-67 IHC exhibiting cautery artifact; these areas should be excluded from the score (20×, objective).

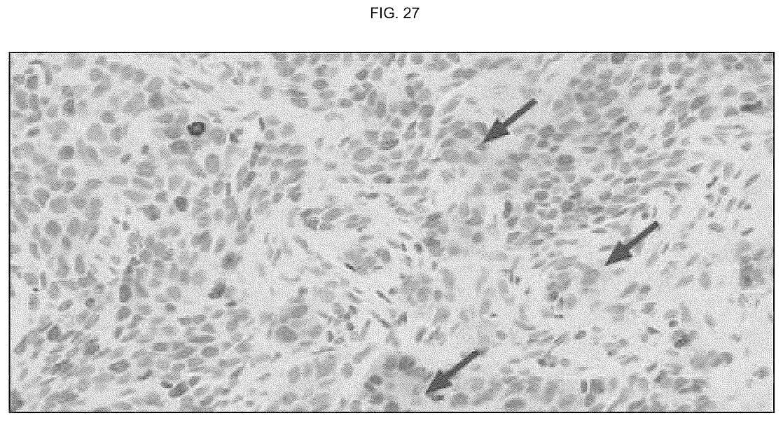

FIG. 27 illustrates an image of a breast carcinoma specimen stained with an exemplary Ki-67 IHC exhibiting acceptable (≤1+) non-specific staining; non-specific background staining (arrows) should be excluded from the score. Weak nuclear staining is also present and should be included (20× objective).

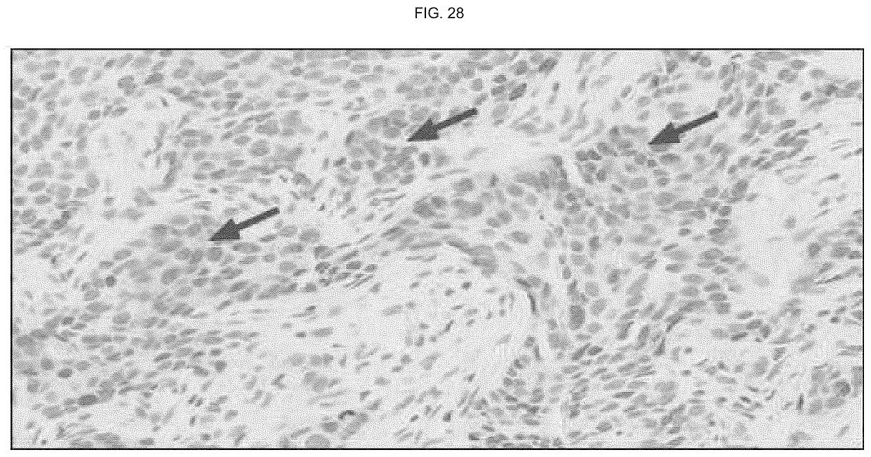

FIG. 28 illustrates an image of a Negative Control Reagent (NCR) exhibiting acceptable (≤1+) non-specific background staining in breast carcinoma (arrows) (20×objective).

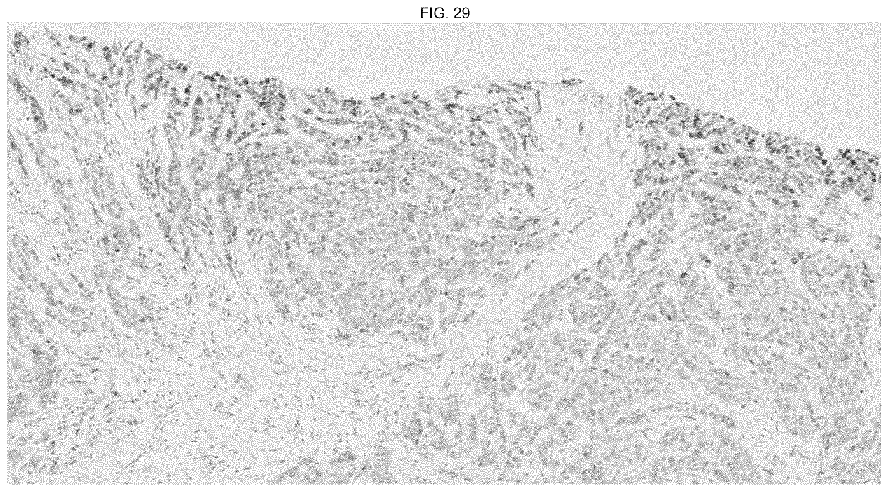

Commonly, edge artifact is linked to the following pre-analytic factors: Thick tissue sections; Tissue cautery; or Drying of tissue prior to fixation or during staining procedure. These factors can lead to accentuation of staining at the periphery of the section. In this case, Ki-67 staining at the edge of the tissue section is excluded from scoring. FIG. 29 illustrates an image of a breast carcinoma specimen stained with an exemplary Ki-67 IHC edge staining artifact should be excluded from the score (5×objective).

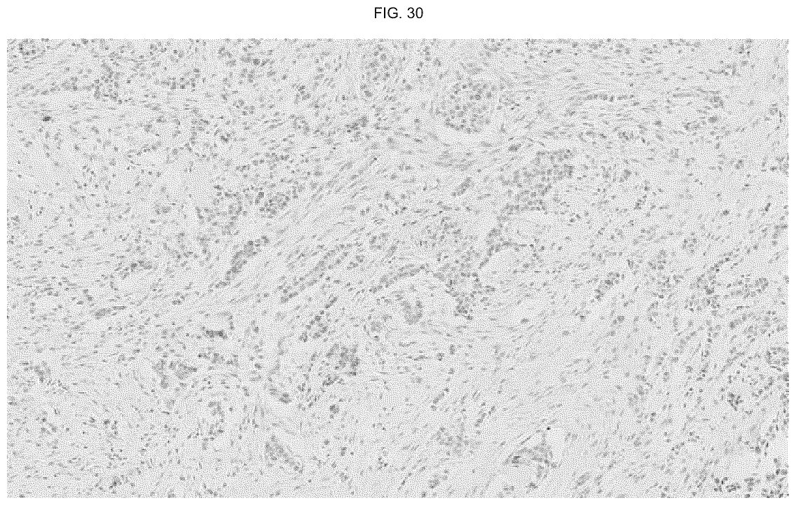

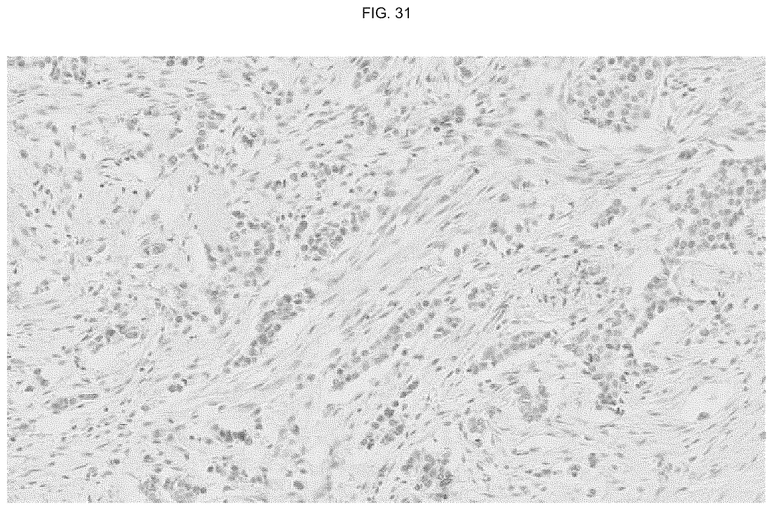

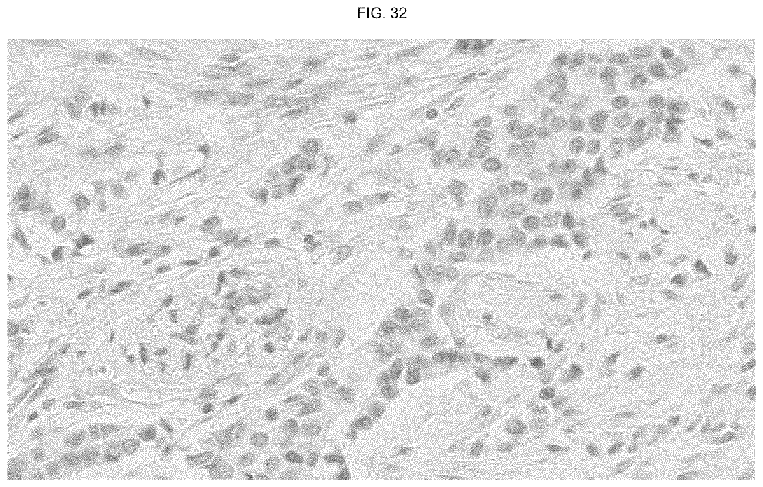

An example of a diagnostic Negative Case (Ki-67 Score less than (<) 20%) is illustrated in FIG. 30 (10× objective), FIG. 31 (20× objective) and FIG. 32 (40×objective), where a breast carcinoma specimen was stained with an exemplary Ki-67 IHC, and the specimen exhibited a Ki-67 Score (%) of 0% (10-40× objective).

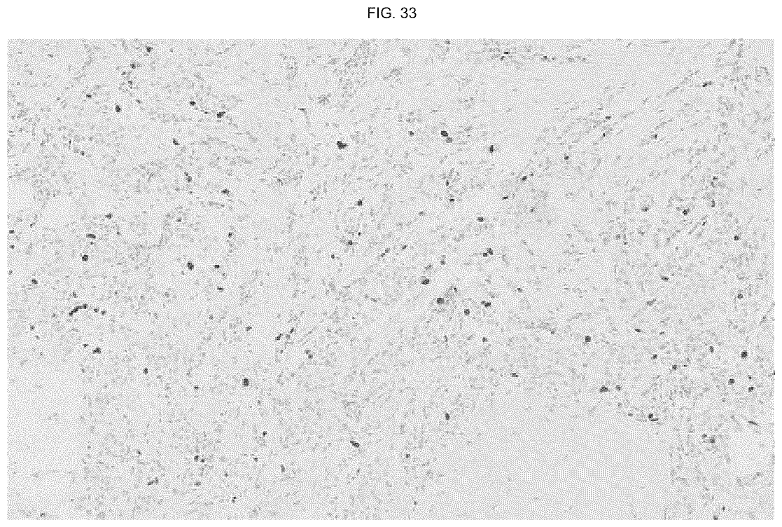

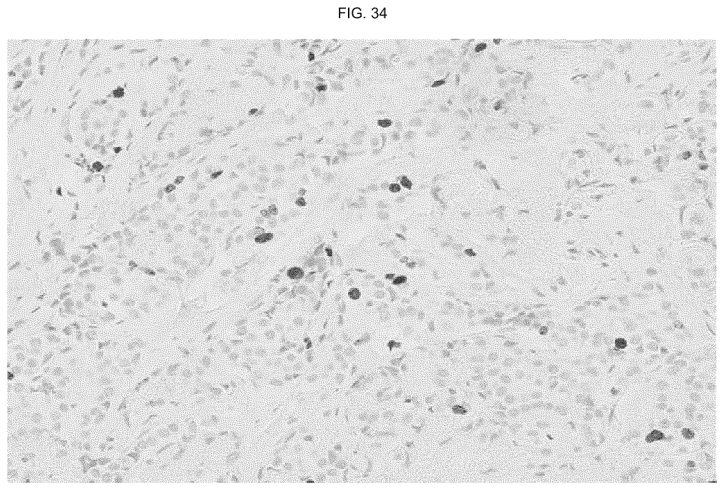

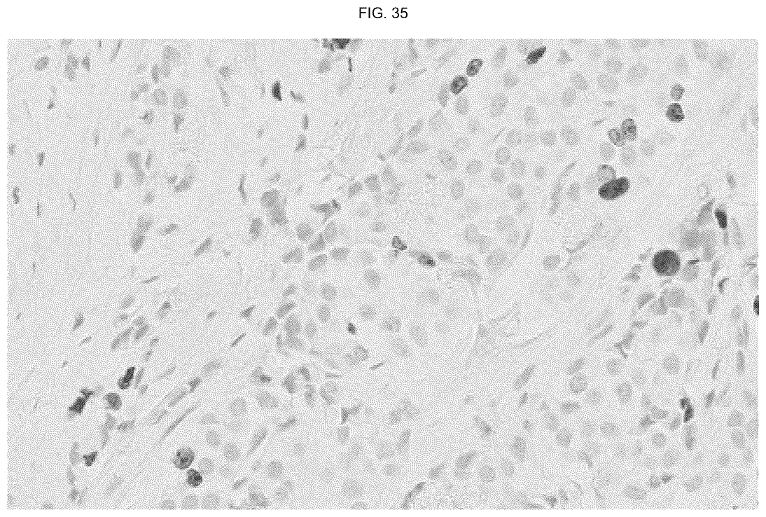

Another example of a diagnostic Negative Case (Ki-67 Score less than (<) 20%) is illustrated in FIG. 33 (10× objective), FIG. 34 (20× objective) and FIG. 35 (40× objective), where a breast carcinoma specimen was stained with an exemplary Ki-67 IHC, and the specimen exhibited a Ki-67 Score (%) of 7% (10× to 40× objective).

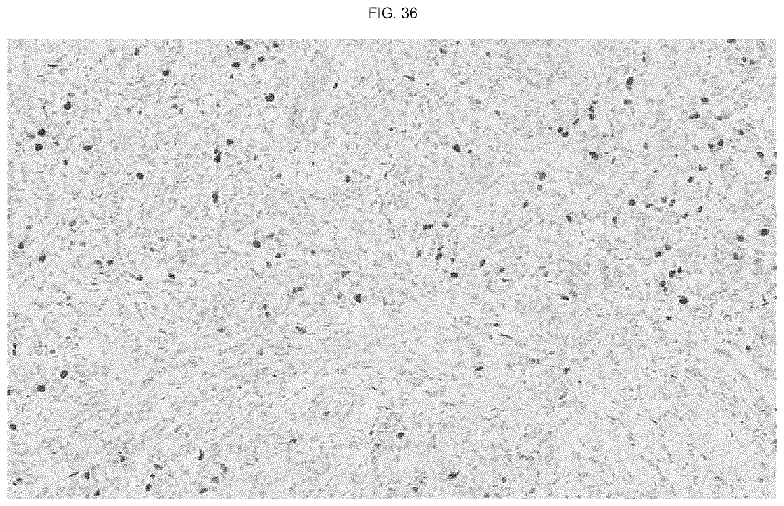

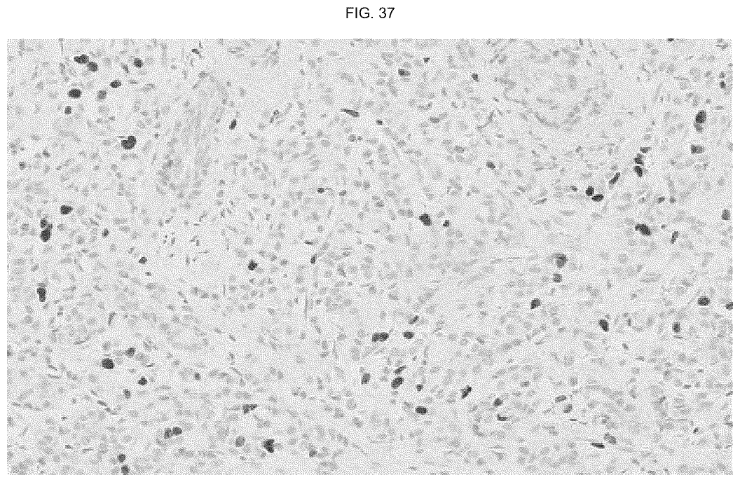

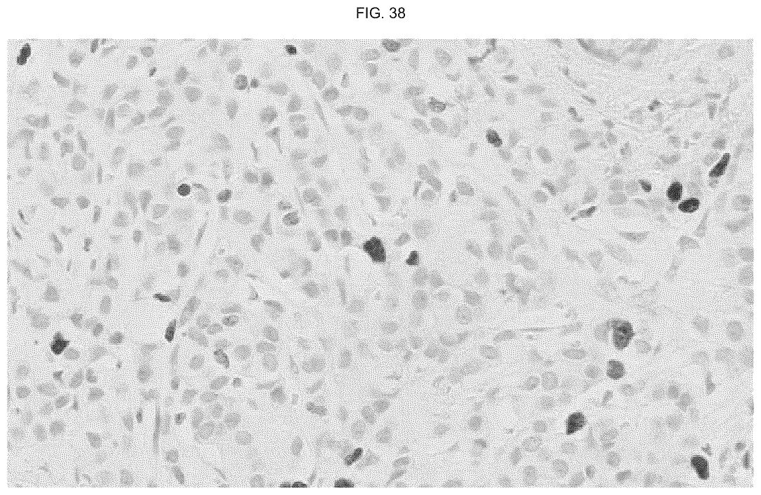

Another example of a diagnostic Negative Case (Ki-67 Score (%) less than (<) 20%) is illustrated in FIG. 36 (10× objective), FIG. 37 (20× objective) and FIG. 38 (40× objective), where a breast carcinoma specimen was stained with an exemplary Ki-67 IHC, and the specimen exhibited a Ki-67 Score of 11% (10-40× objective).

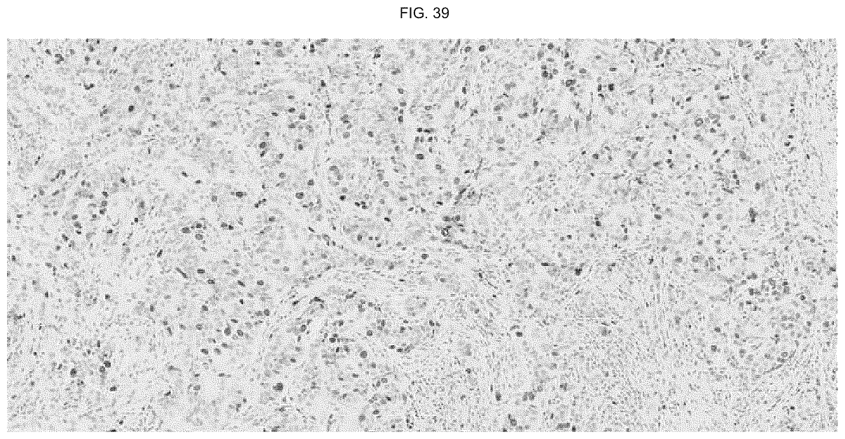

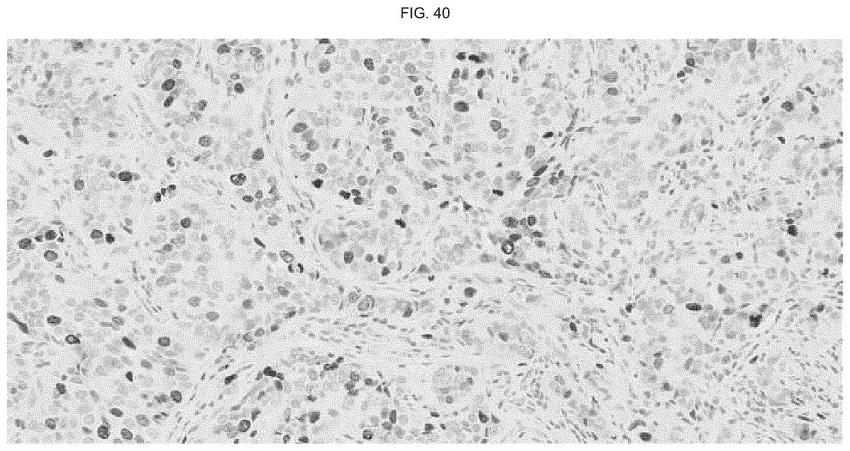

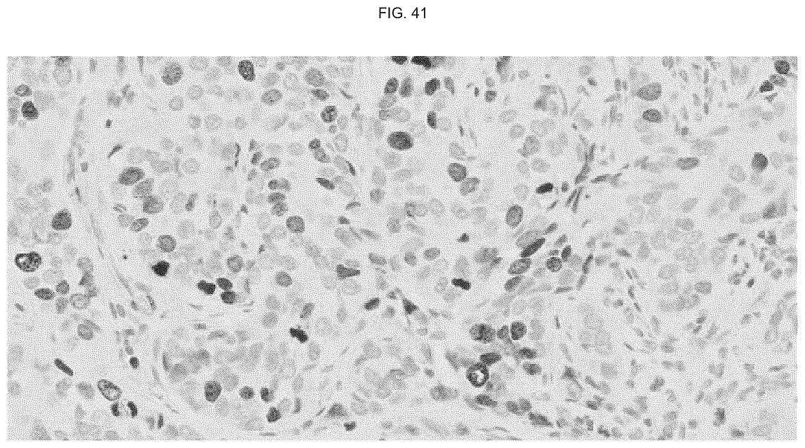

An example of a diagnostic positive case (Ki-67 Score (%) greater than or equal to (≥) 20%) is illustrated in FIG. 39 (10× objective), FIG. 40 (20× objective) and FIG. 41 (40× objective), where a breast carcinoma specimen was stained with an exemplary Ki-67 IHC, and the specimen exhibited a Ki-67 Score (%) of 43% (10-40× objective).

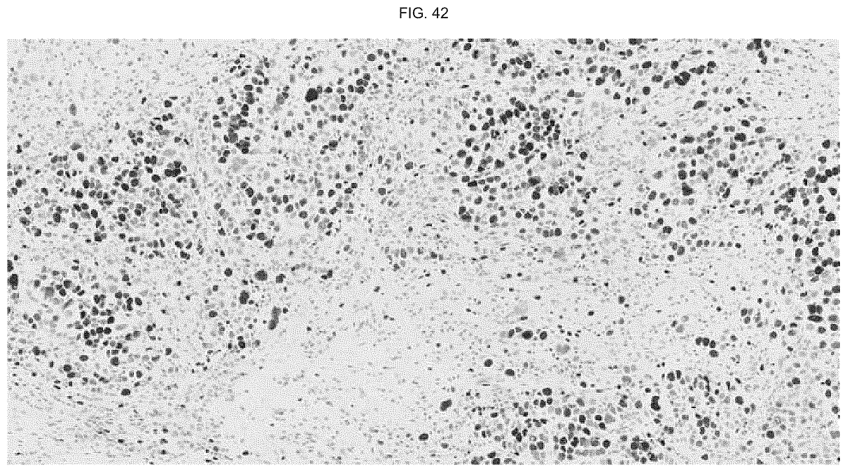

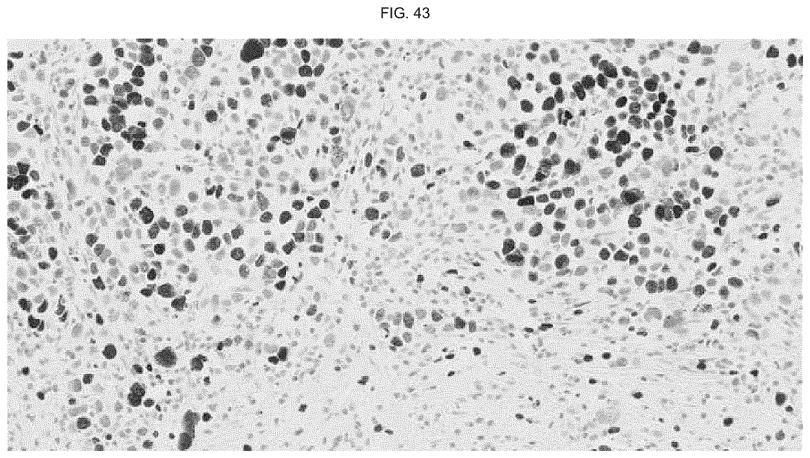

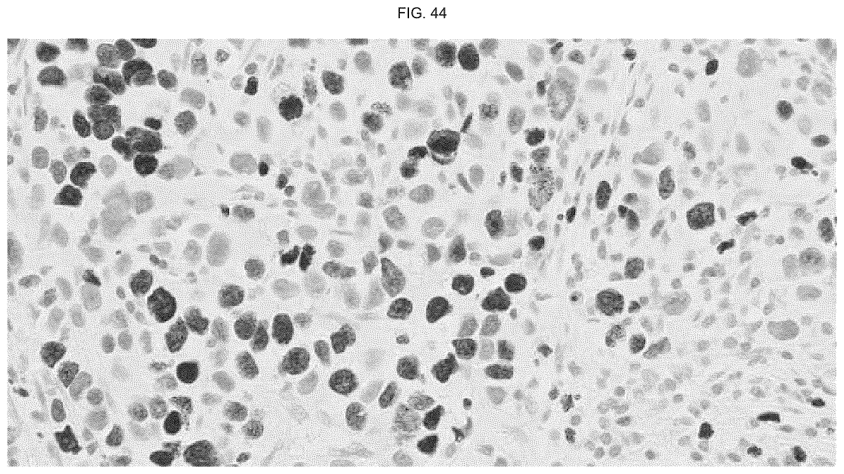

Another example of a diagnostic positive Case (Ki-67 Score (%) greater than or equal to (≥) 20%) is illustrated in FIG. 42 (10× objective), FIG. 43 (20× objective) and FIG. 44 (40× objective), where a breast carcinoma specimen was stained with an exemplary Ki-67 IHC, and the specimen exhibited a Ki-67 Score (%) of 52% (10-40× objective).

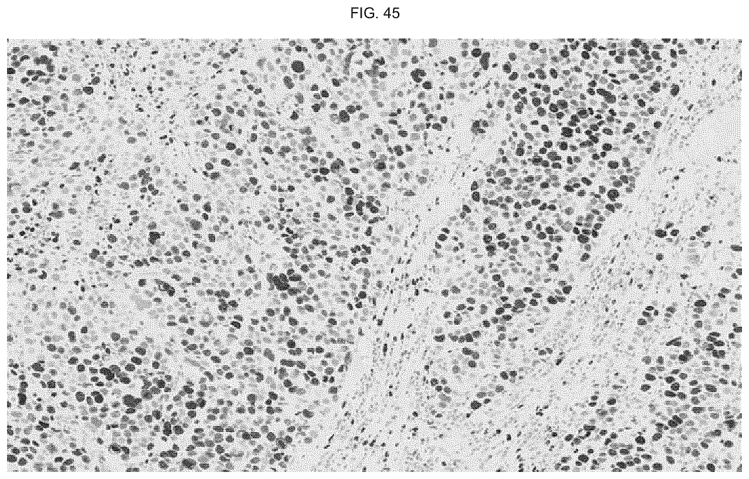

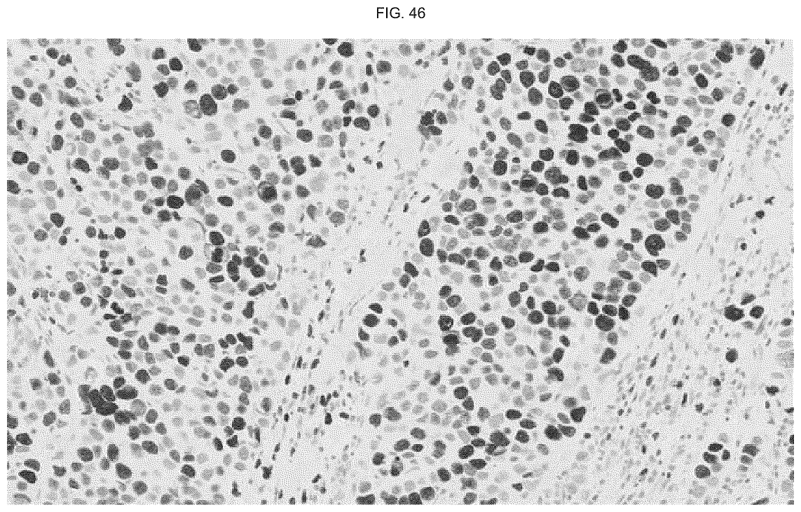

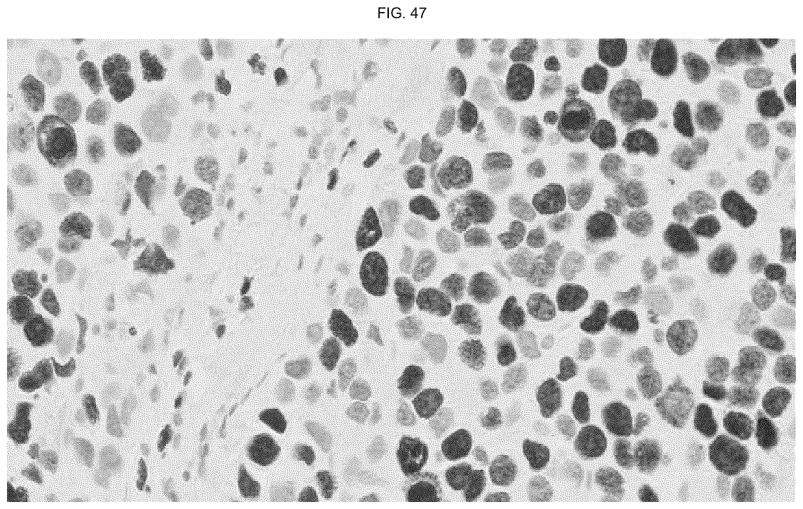

Another example of a diagnostic positive Case (Ki-67 Score (%) greater than or equal to (≥) 20%) is illustrated in FIG. 45 (10× objective), FIG. 46 (20× objective) and FIG. 47 (40× objective), where a breast carcinoma specimen was stained with an exemplary Ki-67 IHC, and the specimen exhibited a Ki-67 Score (%) of 82% (10-40× objective).

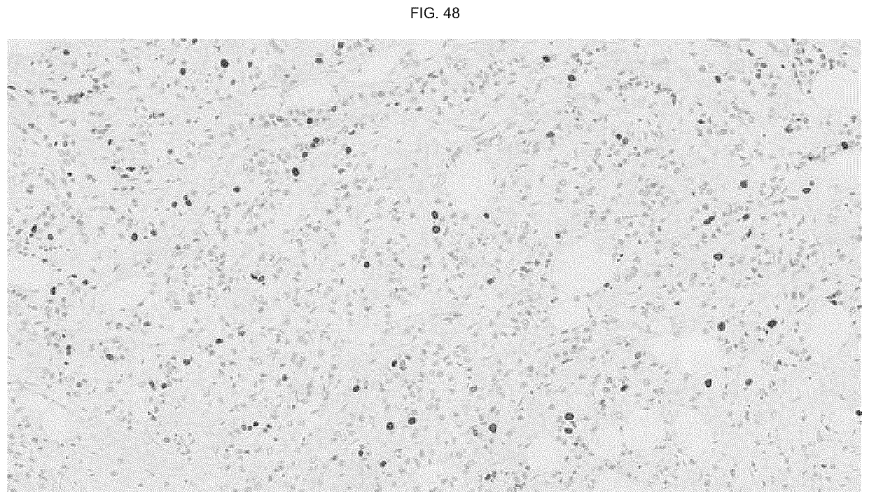

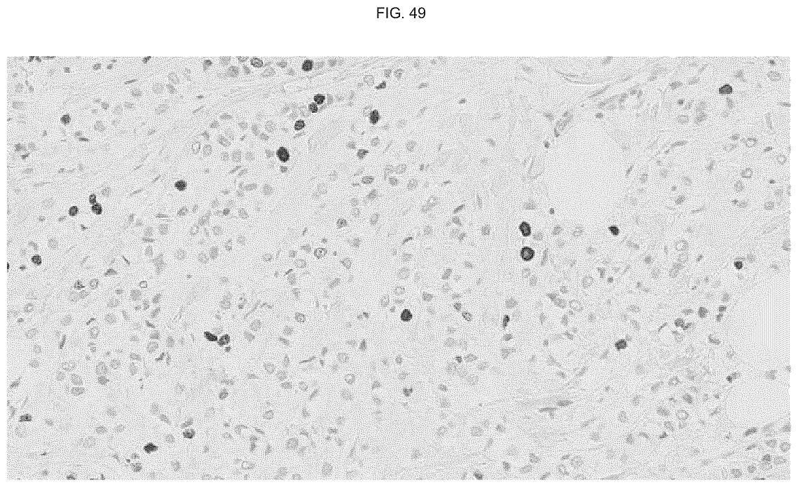

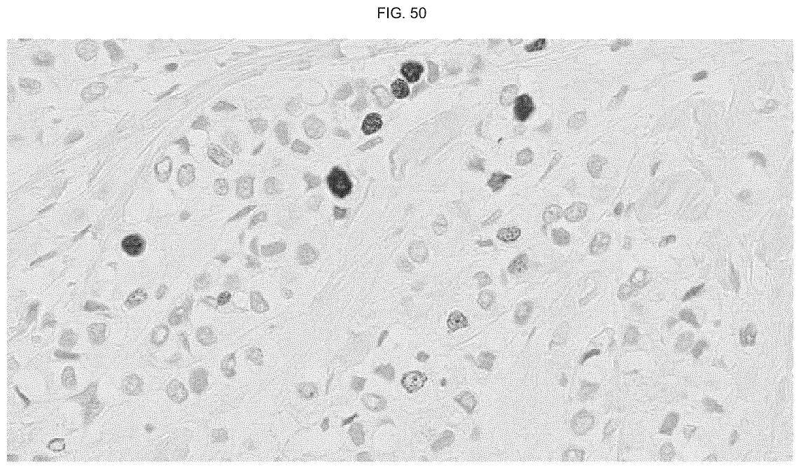

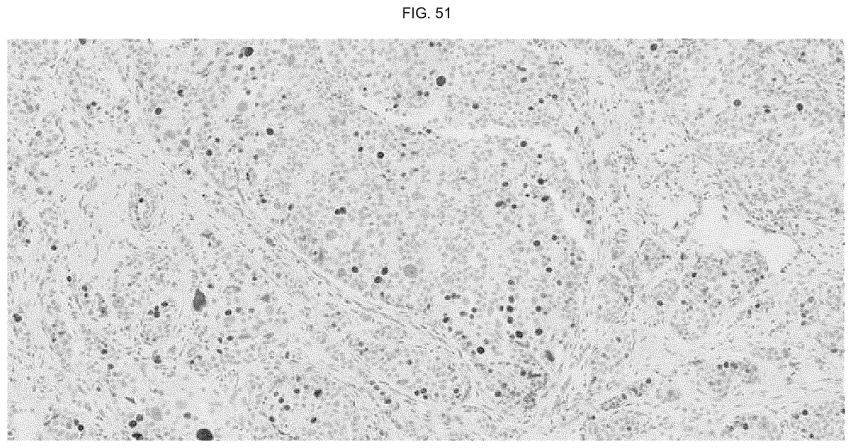

An example of a “near cut-off” but diagnostic negative case (Ki-67 Score (%) Range of greater than or equal to (≥) 10% but less than (<) 20%) is illustrated in FIG. 48 (10× objective), FIG. 49 (20× objective) and FIG. 50 (40× objective), where a breast carcinoma specimen was stained with an exemplary Ki-67 IHC, and the specimen exhibited a Ki-67 Score (%) of 15% (10-40× objective).

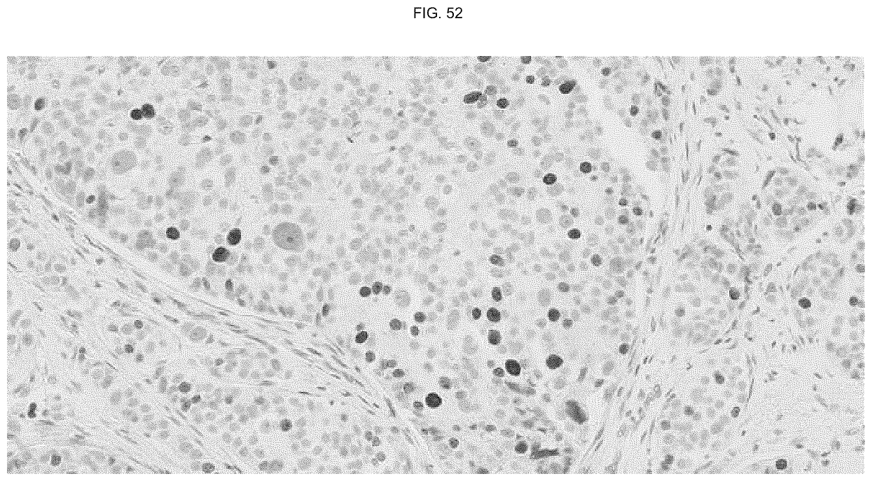

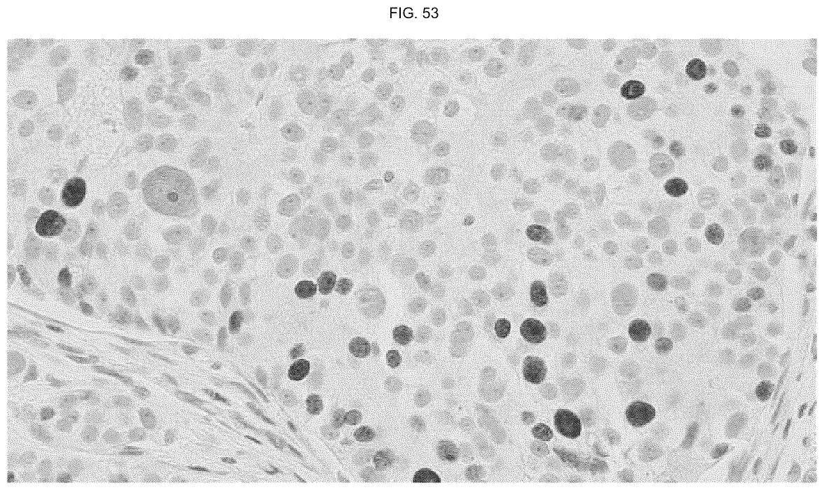

An example of a “near cut-off” but diagnostic negative case (Ki-67 Score (%) Range of greater than or equal to (≥) 10% but less than (<) 20%) is illustrated in FIG. 51 (10× objective), FIG. 52 (20× objective) and FIG. 53 (40× objective), where a breast carcinoma specimen was stained with an exemplary Ki-67 IHC, and the specimen exhibited a Ki-67 Score (%) of 15% (10-40× objective).

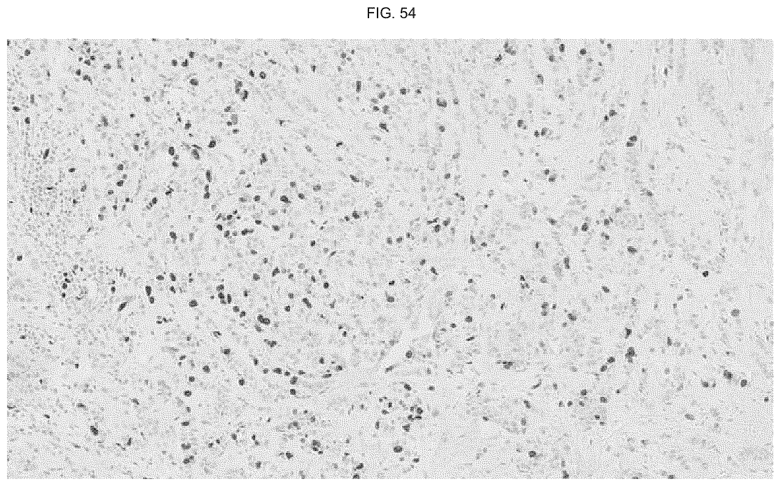

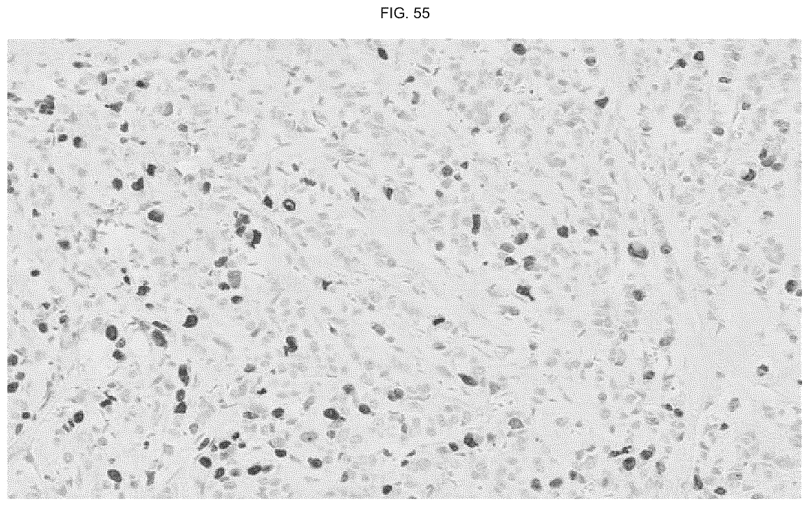

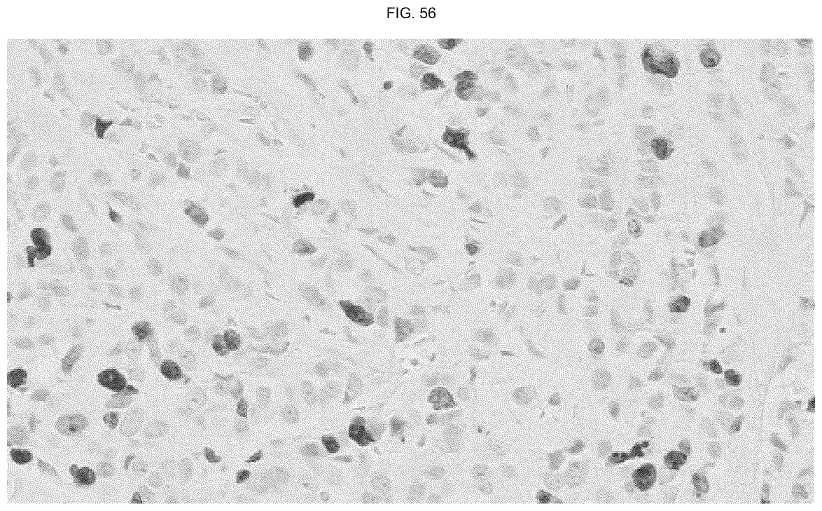

An example of a “near cut-off” but diagnostic positive case (Ki-67 Score (%) Range of greater than or equal to (≥) 20% but less than or equal to (≤) 30%) is illustrated in FIG. 54 (10× objective), FIG. 55 (20× objective) and FIG. 56 (40× objective), where a breast carcinoma specimen was stained with an exemplary Ki-67 IHC, and the specimen exhibited a Ki-67 Score (%) of 21% (10× to 40× objective).

### Example 2: Use of Ki-67 Scores (%) as a Companion or Complementary Diagnostic for Breast Cancer Treatment

In alternative embodiments, provided are methods for treating or ameliorating a cancer or a tumor in an individual in need thereof, comprising: determining if cells in a tissue sample from a patient have a low or a high, or a diagnostic negative or a diagnostic positive, Ki-67 expression score, as determined by a protocol comprising use of an immunohistochemistry (IHC) method as provided herein; followed by administering the tumor or cancer therapeutic such as a therapeutic developed to inhibit CDK4 and CDK6, such as for example: abemaciclib, palbociclib (optionally IBRANCE™ or PALBONIX™), or ribociclib (optionally KISQUALI™) to the individual if the tissue sample is found to have a high or a diagnostic positive Ki-67 expression score (%).

In alternative embodiments, Ki-67 IHC methods as provided herein are used in a randomized, open-label, phase 3 study of abemaciclib combined with standard adjuvant endocrine therapy versus standard adjuvant endocrine therapy alone in patients with high risk, node positive, early stage, hormone receptor positive, human epidermal receptor 2 negative, breast cancer.

Abemaciclib is administered orally and standard adjuvant endocrine therapy administered according to package label. Standard adjuvant endocrine therapy is administered according to package label.

The primary outcome measure is invasive disease free survival (IDFS).

In alternative embodiments, secondary outcome measures can be used, which can be:
DFS for participants with a Ki-67 score (%) greater than or equal to (≥) 20%;
Distant Relapse-Free Survival (DRFS);
Overall Survival (OS);
Pharmacokinetics (PK): Minimum Steady State Concentration (Cmin,ss) of abemaciclib;
Change from Baseline on the Functional Assessment of Cancer Therapy—Breast (FACT-B);
Change from Baseline on the Functional Assessment of Cancer Therapy—Endocrine Symptoms (FACT-ES);
Change from Baseline on the Functional Assessment of Chronic Illness Therapy—Fatigue (FACIT-F); and,
Change from Baseline on the EuroQol Five-Dimension Five-Level Questionnaire (EQ-5D-5L).

### Eligibility Criteria:

### Exemplary IHC Inclusion Criteria:

The participant has confirmed HR+, HER2−, early stage resected invasive breast cancer without evidence of distant metastases.
The participant must have undergone definitive surgery of the primary breast tumor.
Pathologic lymph node involvement and at least one of the following indicating a higher risk of recurrence:
4 or more positive axillary lymph nodes
Tumor size of at least 5 centimeters
Grade 3 defined as at least 8 points on the Bloom Richardson grading system
Ki-67 score (%) by central analysis of greater than or equal to (≥) 20% on untreated breast tissue
The participant has a Eastern Cooperative Oncology Group (ECOG) performance status less than or equal to (≤1).
   Exemplary Exclusion Criteria, one or more of these may be considered:
Metastatic disease (including contralateral axillary lymph nodes) or node-negative disease.
Participants with inflammatory breast cancer.
Participants with a history of previous breast cancer, with the exception of ipsilateral ductal carcinoma in situ (DCIS) treated by locoregional therapy alone greater than or equal to (≥) 5 years ago. Participants with a history of contralateral DCIS treated by local regional therapy at any time may be eligible. Participants with a history of any other cancer (except non-melanoma skin cancer or carcinoma in situ of the cervix), unless in complete remission with no therapy for a minimum of 5 years from the date of randomization are excluded.

It is observed that subjects with a Ki-67 score (%) of 20% or greater respond favorably to treatment with abemaciclib. Similar assessments may be made for Ki-67 scores having thresholds other than 20%, for example thresholds of about 1%, about 5%, about 10%, about 20%, about 30%, about 40%, about 50% or about any number between 1% and 50%.

It is determined that subjects with Ki-67 scores (%) above the threshold respond favorably to treatment with abemaciclib, thus demonstrating that Ki-67 scores (%) at or above the threshold can be used to guide treatment with abemaciclib.

Similar analyses may be conducted with other cyclin dependent kinase inhibitors to identify Ki-67 scores (%) indicative of a favorable response to treatment with these therapeutics.

### Example 3: Sensitivity, Specificity, Precision, and Robustness of Exemplary Ki-67 IHC Assay

In alternative embodiments, provided are methods for determining and reproducibly scoring the extent of nuclear expression of protein Ki-67 in a tissue sample. In alternative embodiments, methods as provided herein address the lack of standardized Ki-67 assay performance, which before this invention remained an impairment to global clinical research (Dowsett M et al.: Assessment of Ki67 in Breast cancer: Recommendations from the International Ki67 in Breast Cancer Working Group. JNCI 103:1656-64. 2011). IHC assays and scoring algorithms as provided herein address this problem and can reproducibly detect Ki-67 expression in formalin-fixed paraffin-embedded human breast carcinoma.

### Methods:

In alternative embodiments the Ki-67 scoring (%) assay is based on EnVision FLEX™ technology using anti-Ki-67 clone MIB-1. The exemplary scoring guide as provided herein was developed and optimized for high inter-observer precision. This exemplary assay has been analytically validated for sensitivity, specificity, precision (inter-day, inter-instrument, inter-lot and repeatability: intra-instrument/intra-rack/intra-day) and robustness.

### Results:

The exemplary Ki-67 IHC detected a relevant range of Ki-67 expression in 148 breast carcinoma specimens, including resection and core needle biopsy specimens. All precision and robustness studies achieved 95% confidence interval lower bounds (LBCI) of greater than (>) 90% for negative percent (NPA), positive percent (PPA) and overall (OA) agreement. Specifically, observer reproducibility results demonstrated high agreement with 95% LBCI values for NPA/PPA/OA of: 97.2/a 91.7%/95.4% for inter-observer and 98.3%/94.4%/96.8% for intra-observer.

### Conclusions

These studies demonstrate that the exemplary standardized Ki-67 IHC assay as provided herein are sensitive, specific, precise, and robust for reproducible detection of Ki-67 expression in breast carcinoma. In some embodiments, the Ki-67 IHC assays described herein may be utilized on the Dako OMNIS™ platform.

### Example 4: Reproducibility of an Exemplary Ki-67 IHC Assay

In alternative embodiments, provided are methods for determining and reproducibly scoring the extent of nuclear expression of protein Ki-67 in a tissue sample. While Ki-67 immunohistochemistry (IHC) is a widely used tumor proliferation assessment, considerable variation exists in assay format and interpretation. The reproducibility of an exemplary analytically validated Ki-67 IHC assay as provided herein was evaluated in an abemaciclib adjuvant early breast cancer study.

Design: A blinded, randomized study was conducted in two Parts. Part A assessed inter-/intra-site reproducibility of staining and Ki-67 interpretation of 5 replicate sets of 30 formalin-fixed, paraffin-embedded breast carcinoma specimens performed at three external laboratories. Part B assessed inter-/intra-observer reproducibility with one pre-stained set consisting of 60 formalin-fixed, paraffin-embedded breast carcinoma specimens. A balanced sample distribution was used with a washout period of 14 to 30 calendar days between evaluations. Reproducibility was measured using diagnostic endpoints: “positive” if Ki-67-expressing tumor cells were greater than or equal to (≥) 20% and “negative” if less than (<) 20%. Negative percent agreement, positive percent agreement, and overall percent agreement with two-sided 95% confidence intervals were calculated. Acceptance criteria for all agreements required that the two-sided 95% confidence interval lower-bound values must be ≥85%.

Results: The calculated lower-bounds of the 95% confidence intervals were greater than or equal to (≥) 85% for all parameters at the greater than or equal to (≥) 20% cut-off. Inter-site reproducibility across three sites, intra-site/inter-day reproducibility across five days within three sites, inter-observer reproducibility across three pathologists, and intra-observer reproducibility across three reads by three pathologists met all acceptance criteria.

Conclusions: The analysis demonstrated robust external reproducibility results at the greater than or equal to (≥) 20% cut-off and support the use of exemplary Ki-67 IHC assay as provided herein for reproducible detection of Ki-67 in breast carcinomas.

### Example 5: Validation of an Exemplary Ki-67 IHC Assay

In alternative embodiments, provided are methods for determining and reproducibly scoring the extent of nuclear expression of protein Ki-67 in a tissue sample, an exemplary scoring method, a Ki-67 scoring (%) as provided herein, referred to in this Example as “the Ki-67 score”.

Context—An abemaciclib adjuvant early breast cancer study demonstrated positive efficacy results during a pre-defined interim analysis of 5,637 high-risk early breast cancer patients. Secondary outcome measures included evaluation of invasive disease-free survival for participants with Ki-67 greater than or equal to (≥) 20%.

Immunohistochemistry (IHC) evaluation for Ki-67 is a common analysis method for assessing cellular proliferation status, but there is a lack of standardized procedures and accepted cut-off definitions for Ki-67.

Objective—To develop a standardized approach for Ki-67 THC and evaluate multi-site reproducibility of a novel, fully validated Ki-67 IHC scoring method as provided herein.

Design—The exemplary assay as provided herein has been analytically validated for sensitivity, specificity, repeatability, precision (inter-instrument, inter-day, inter-lot), and robustness (target retrieval and tissue thickness) using the greater than or equal to (≥) 20% cut-off. Reproducibility studies (inter- and intra-site, inter- and intra-observer) were conducted at 3 external laboratories using the same cut-off.

Results—All analytical validation studies achieved point estimates of greater than 90% for negative, positive, and overall percent agreement. Inter-site reproducibility demonstrated point estimate values of 94.7%, 100.0%, and 97.3%, respectively, and external inter-observer reproducibility demonstrated point estimate values of 98.9%, 97.8%, and 98.3%, respectively.

Conclusions—Standardization of the Ki-67 scoring methodology using methods as provided herein, as well as the pre-analytical and analytical variables, resulted in high concordance in staining and scoring across multiple laboratories. Standardized Ki-67 scoring assays as provided herein can aid treatment decisions in patients with early breast carcinoma where Ki-67 expression levels are involved in prognosis, risk of disease recurrence, or prediction of response to therapy.

Ki-67 antigen, also known as Ki-67 or MK167, is a nuclear protein associated with cellular proliferation. The protein is expressed during the active cell cycle phases (G₁, S, G₂, and M phase), but not in the quiescent G₀ phase. Dysregulated proliferation is a distinct characteristic of tumors, and Ki-67 has shown to be a promising biomarker candidate in aggressive lesions. A Ki-67 labeling index has been found to correlate with tumor grade and clinical course in many cancer types. In the context of breast carcinoma, the percentage of Ki-67 positive cells allows for classification between luminal A and B type tumors, and high Ki-67 index values have been associated with breast carcinomas that are large, high grade, positive for lymph node involvement, and either triple negative or human epidermal growth factor receptor-2 (HER2) positive.

The measurement of Ki-67 expression has gained considerable attention as a possible predictive marker of responsiveness or resistance to chemotherapy or endocrine therapy. However, there are practical limitations to the value of some Ki-67 study results due to substantial heterogeneity and variable validity in the pre-analytical and analytical methods used. The lack of standardized procedures or accepted cut-off definitions for Ki-67 has prohibited comparisons of Ki-67 data between clinical trials and limited the application of Ki-67 assessment for clinical use. Therefore, Ki-67 immunohistochemistry (IHC) is not routinely performed as part of the diagnostic work up of breast carcinomas in the clinical setting of many geographies, and the impact of Ki-67 detection in the management of patients with breast carcinoma remains to be universally accepted. In an effort to minimize the variability and facilitate adoption of Ki-67 assessments in the clinic, an expert group on Ki-67 testing in breast carcinoma has provided guidance on preferred methods for Ki-67 staining and scoring (See for example, Dowsett M, et al., Assessment of Ki67 in breast cancer: recommendations from the International Ki67 in Breast Cancer working group, J Natl Cancer Inst. 2011; 103(22):1656-1664).

Cyclin-dependent kinase 4 and 6 (CDK4 and 6) inhibitors have improved outcomes for patients with hormone receptor positive (HR+), HER2-negative (HER2−) advanced breast cancer when used in combination with endocrine therapy. Abemaciclib is a selective CDK4 and 6 inhibitor approved for the treatment of HR+, HER2−advanced or metastatic breast cancer. A neoadjuvant study of abemaciclib and anastrozole in HR+, HER2−breast cancer incorporated the International Ki-67 in Breast Cancer working group guidelines when defining the change in Ki-67 measurement from baseline to 2 weeks post-treatment as a primary study outcome. The study met its primary endpoint by demonstrating a greater decrease in Ki-67 tumor expression following treatment with abemaciclib alone, or in combination with anastrozole, compared with anastrozole alone. Ki-67 measurements of cell cycle inhibition inform the biological risk hypothesis and support assessing the impact of tumor proliferation in the setting of early breast cancer with a high-risk of recurrence. To enable a global multi-site registrational study design, a standardized automated testing system and uniform scoring algorithm were developed to detect Ki-67 expression in formalin-fixed, paraffin-embedded human breast carcinoma.

The present analysis was conducted in support of an abemaciclib adjuvant early breast cancer study and evaluates abemaciclib adjuvant therapy in high-risk, node positive, early, HR+, HER2−breast cancer patients evaluates abemaciclib adjuvant therapy in high-risk, node positive, early, HR+, HER2−breast cancer patients.

In alternative embodiment, provided herein is a standardized Ki-67 assay that can be used to aid treatment decisions in patients with early breast carcinoma where Ki-67 expression levels are involved in prognosis, risk of disease recurrence, or prediction of response to therapy.

### Materials and Methods

### Tissue Specimen Preparation

Unless otherwise noted, specimens used in these studies were commercially procured formalin-fixed, paraffin-embedded (FFPE) human breast carcinoma tissue. Specimens consisted of both core biopsies and surgical resection tissue as indicated. Information regarding the HR status and HER2 status was not available for all specimens. Sections were cut at 4-5 μm thickness, placed on positively charged slides, and dried in an oven at 58° C.±2° C. for 1 hour. The mounted sections were stored in the dark at 2° C. to 8° C. and stained using an exemplary Ki-67 IHC assay described herein within 5 months of sectioning. Tonsil tissues were used as an assay control and for evaluation of pre-analytical variables.

### Prototype Assay Design Input

Early input for the assay prototype was provided from a qualitative comparison of commercially acquired FFPE human breast carcinoma tissue assessed by the Ki-67 IHC assay used in the neoadjuvant study of abemaciclib and anastrozole in HR+, HER2− breast cancer. A limited sample set of 18 invasive breast carcinoma resection specimens, a tissue microarray comprised of 49 invasive breast carcinoma cores, and tonsil control tissue were assayed with the laboratory developed test (LDT) at University of Southern California, and were assayed with an exemplary Ki-67 IHC assay described herein. Alternating section levels were stained with both assays prior to performance comparisons. The study was performed for informational purposes only, and no formal acceptance criteria were applied.

### Exemplary Ki-67 IHC Assay

THC staining procedure was performed on the Dako OMNIS™ platform and used an automated staining protocol validated for the Ki-67 IHC assay described herein. Ki-67 IHC is a modular assay consisting of optimized purified monoclonal mouse antibody (clone MIB-1), produced in a good manufacturing practice environment under strict quality control criteria with regards to purity, integrity and concentration, and an isotype-control antibody with matched protein concentration. Accessory system reagents, required to complete an IHC staining procedure, are available in individual packaging from Agilent Technologies, Inc., Santa Clara, CA. Table 4 provides a summary of relevant factors incorporated in the development of the Ki-67 IHC assay described herein.

Heat-induced epitope retrieval was performed using diluted EnVision FLEX Target Retrieval Solution, Low pH (50×) (Dako OMNIS™) (Code GV805). Deparaffinization, rehydration, and target retrieval was performed on-board the Dako OMNIS™. After incubation with the primary monoclonal mouse anti-human Ki-67 antibody, clone MIB-1, or the Negative Control Reagent (NCR; mouse immunoglobulin G isotype control), specimens were incubated with ready-to-use visualization reagent consisting of a secondary antibody and horseradish peroxidase coupled to a dextran polymer backbone (Dako OMNIS™; Code GV800, or respective bulk reagents). The enzymatic conversion of the subsequently added 3,3′-diaminobenzidine tetrahydrochloride chromogen resulted in precipitation of a visible reaction product at the site of antigen. The specimens were then counterstained with Hematoxylin (Dako OMNIS™; Code GC808) and coverslipped. All reagents and instruments were manufactured and supplied by Dako North America. Glass slides were scored as described under “Scoring Interpretation”.

### Scoring Interpretation

Ki-67 IHC assay results were interpreted using a light microscope. A minimum of 200 viable invasive tumor cells was required for scoring. All viable invasive tumor cells in the specimens were evaluated and included in the Ki-67 scoring assessment. Carcinoma in situ was not scored. Only nuclear staining was considered for evaluation of positive staining in tumor cell nuclei. A tumor cell was considered positive when the signal was unequivocally brown and covered the whole chromatin distribution within the nucleus. For determination of Ki-67 protein expression, intensity grades of 1+(weak staining) to 3+(strong staining) were reported. Nonspecific staining was recorded using a 0 to 3+ scale, in 0.25 increments. Cytoplasmic and/or membrane staining, if present, was excluded from the scoring. The Ki-67 score was determined as the number of Ki-67 staining viable invasive tumor cells divided by the total number of viable invasive tumor cells, multiplied by 100. Breast carcinoma specimens stained with the NCR were required to have less than (<) 1+ intensity nonspecific background staining, in order for the same specimens stained with the Ki-67 antibody to be considered valid. Tumors were classified as diagnostic positive or negative based on a greater than or equal to (≥) 20% cut-off, with a Ki-67 score of greater than or equal to (≥) 20% being considered as positive and less than (<) 20% being considered negative. An overview of the scoring methodology is provided in the post-analytical (Interpretation and Scoring) section of Table 6.

Ki-67 IHC immunostaining results were assessed through blinded and randomized slide evaluation in all studies except for the 31 Normal Tissue testing and the Pre-Analytical Variables studies.

Internal observers and external laboratory pathologists were trained and tested on the scoring algorithm/guidelines. In the initial project phase, hot spots were also analyzed in an exploratory fashion. A hot spot was defined as the area corresponding to the field of vision in a 20× objective with the highest percentage of positive tumor nuclei in the section.

### Investigational Assay Sensitivity Study

The expression level of Ki-67 protein in procured FFPE breast carcinoma tissue sections using the exemplary Ki-67 IHC assay was evaluated. One hundred and forty-eight specimens were stained using the exemplary Ki-67 IHC assay. Specimens reflected a range of Ki-67 expression levels and included both resection (n=100) and core needle biopsy (CNB) (n=48) specimens. A subset of the specimens were sister blocks from the same case, therefore prevalence analysis was conducted using the average score from these blocks. Prevalence analysis was performed on 113 unique specimens (resection, n=80: CNB, n=33).

Investigational Assay Specificity studies

### A. Western Blot

Cell lysates from 2 cancer cell lines, SKBR3 and IM-9, were used for a Western immunoblot to establish the specificity of the MIB-1 antibody for Ki-67. The samples were separated by sodium dodecyl sulfate-polyacrylamide gel electrophoresis (SDS-PAGE) along with a protein molecular weight marker ladder (Novex Hi-Mark, Fisher Scientific) and then transferred onto a polyvinylidene difluoride membrane. To show inhibition of Ki-67 MIB-1 antibody binding in the presence of a peptide derived from a region of the Ki-67 protein containing the binding epitope (Abcam 15581), the blot was cut into 3 identical strips, each containing the transferred SKBR3 and IM-9 cell lysate proteins. One strip was incubated with MIB-1 clone alone as primary antibody, the other two with MIB-1 antibody pre-mixed with different amounts of inhibitory peptide (5× and 15× excess by weight). Following primary antibody incubation, Ki-67 protein was detected with a goat anti-mouse fluorescent tagged secondary antibody using SUPERSIGNAL WEST FEMTO™ substrate (Fisher Scientific). After imaging, the blots were stripped of Ki-67 primary antibody and re-stained with glyceraldehyde-3-phosphate dehydrogenase (GAPDH) housekeeping protein antibody to show equal loading of cell lysate proteins. The detection method for the housekeeping protein was a goat anti-mouse fluorescent-tagged secondary antibody.

### B. Immunoreactivity: 31 Normal Tissue Testing

IHC-based specificity testing was conducted on a sample set consisting of resection specimens derived from 3 different cases each of the 30 tissue types recommended by the FDA¹⁶, as well as 3 normal bladder specimens.

### Ki-67 IHC Assay Robustness Study

Ki-67 IHC assay was evaluated under various laboratory conditions (diluted Target Retrieval Solution pH range 5.9-6.5, tissue section thickness from 3-6 μm, and overnight and over-weekend staining procedures) to assess the robustness of the assay.

The effect of pre-analytical variables was also studied. For evaluation of fixative type and fixation time, archived tonsil specimens (n=4 per condition tested in triplicate) that had been processed with various fixatives (i.e. 10% neutral-buffered formalin (NBF), acetic formalin alcohol, Bouin's solution, 10% unbuffered formalin, and 10% NBF followed by 4-5 days in 70% Ethanol) and fixation times (24, 48, or 72 hours) were stained. Additionally, fixation and ischemia times were evaluated on fresh tonsil tissue cut into approximately equally sized pieces and placed under wet gauze at ambient temperature for various ischemia times before being placed into NBF fixative. Fixation times between 6 to 72 hours and ischemia times between 30 minutes and 72 hours were studied. Prospectively collected tonsil tissue processed under these defined conditions were obtained by the Cooperative Human Tissue Network.

### Tumor Heterogeneity Study

Tumor heterogeneity was assessed in FFPE breast carcinoma specimens stained with Ki-67 IHC assay. Intra-block tissue heterogeneity was assessed in 36 specimens between non-serial sections across a span of at least 200 μm. Intra-case heterogeneity was assessed between 25 unique breast carcinoma specimen cases in sister block pairs (consisting of 50 total unique block IDs). Sister blocks are defined as paraffin blocks prepared from the same specimen. The positive/negative diagnostic status of each slide was determined based on the cut-off (or greater than or equal to (≥) 20%). Comparisons to consensus were made using the diagnostic status of each block, and were used to calculate negative percent agreement, positive percent agreement, and overall percent agreement.

### Ki-67 IHC Assay Precision Study

Precision testing was conducted internally at Dako North America to demonstrate that the assay produces consistent results in normal day-to-day testing. Repeatability was measured within instrument, staining rack, and day. Assay precision was measured between Dako OMNIS™ instruments, testing days, and different lots of primary antibody and accessory reagent lots. Since the Dako OMNIS™ platform is fully automated, inter-operator variability has been determined to be negligible. Breast carcinoma specimens with a range of expression with regards to percentage of positive tumor cells were selected for internal analytical precision studies. Efforts were made to include approximately 20 to 25% of specimens considered to be in the near cut-off range (10 to 30%). Repeatability and inter-instrument, inter-day, and accessory reagent inter-lot precision studies were performed using replicates from 32 breast carcinoma specimens. Ki-67 IHC assay inter-lot testing utilized replicates from 40 specimens. Precision testing was conducted over 5 non-consecutive days. The stained slides were assessed for their diagnostic concordance in positivity and negativity of Ki-67 expression using a cut-off of greater than or equal to (≥) 20%.

Inter-observer precision was evaluated by testing scoring reproducibility between 3 trained and certified pathologists, who performed 3 independent Ki-67 evaluations on a set of 60 specimens. Information regarding the HR status and HER2 status was not available for all specimens. The consensus for inter-observer analysis was determined as the majority call for the sample across all 9 observations.

### External Reproducibility Study Across Multiple Sites

Inter- and intra-site reproducibility was conducted at 3 external Clinical Laboratory Improvement Amendments (CLIA)-certified laboratories (referred to as “sites”). One trained and certified technician from each laboratory performed 5 automated IHC staining runs using the exemplary Ki-67 IHC assay over 5 non-consecutive days. Each staining run contained replicate sections from the same set of breast carcinoma specimens (n=30), with 1 slide stained with the NCR and 1 slide stained with the Ki-67 primary antibody. Efforts were made to balance the proportion of positive greater than or equal to (≥) 20% and negative less than (<) 20% specimens as well to include approximately 20 to 25% of specimens considered to be in the near cut-off range (10-30%). Each set of blinded and randomized replicate sections was evaluated by a single trained and certified pathologist at each of the 3 external sites with a minimum washout period of 14 days between each evaluation.

Inter- and intra-observer reproducibility across pathologists from different laboratories was assessed through blinded and randomized slide evaluation at 3 external CLIA-certified laboratories. Samples were pre-stained with the exemplary Ki-67 IHC assay at Dako North America and sent to 3 external sites for evaluation. Efforts were made to balance the proportion of positive/negative specimens and include approximately 20 to 25% of specimens considered to be in the near cut-off range. One trained and certified pathologist at each site performed 3 independent Ki-67 staining evaluations on the same set of breast carcinoma specimens (n=60) representing a dynamic range of Ki-67 expression. A minimum washout period of 14 days between each read was implemented. The pathologists participating in the external inter- and intra-observer study were different from the ones scoring the inter- and intra-site reproducibility study.

### Statistical Analysis

Comparisons between the IHC status (diagnostic positive/negative) of each test condition and the consensus (most frequently occurring diagnostic observation within a specimen) were made for each specimen. The pooled comparisons to consensus were then used to calculate agreement parameters. Negative percent agreement (NPA), positive percent agreement (PPA), and overall percent agreement (OA) were calculated for each inter-day, inter-instrument, inter-lot, repeatability, robustness, and reproducibility study, with corresponding 2-sided 95% percentile bootstrap confidence intervals (Cis). In the case where a given parameter (NPA, PPA, and/or OA) resulted in zero discordant comparisons, the Wilson score method was used to compute Cis. Locally estimated scatterplot smoothing (LOESS), a non-parametric regression method, was applied to external inter-observer reproducibility data to evaluate trends in scoring of specimens across the dynamic range of Ki-67 expression. LOESS curves were used to compare the scoring trends across multiple observers as well as across multiple reads within each observer.

### Results

### Definition of Positive Ki-67 Staining

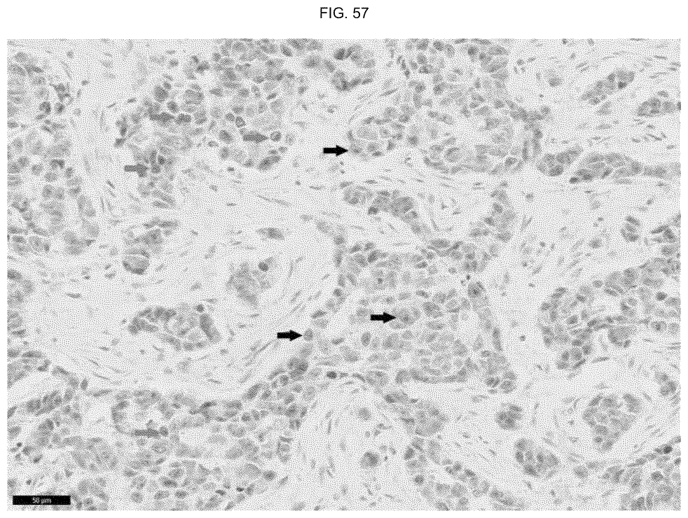

To achieve a high degree of scoring reproducibility, clear and comprehensive scoring guidelines for a dichotomous cut-off of greater than or equal to (≥) 20% positively stained invasive tumor cells were developed. Since any staining intensity greater than (>) 1+ is considered positive, the distinction between staining intensity 0 and 1+ was a critical task. Therefore, specific focus was placed to define and train the recognition of the lower threshold of staining. When assessing if tumor cells were negative versus weakly positive, a cell that exhibited a “grey” coloring in the nucleus was deemed as insufficiently meeting the lower threshold and excluded from the Ki-67 score (FIG. 57). In addition, a detailed description of potential artifacts that can skew results if not managed in a defined approach was provided during pathologist training.

Prototype Comparison with the Neoadjuvant Study of Abemaciclib and Anastrozole in HR+, HER2−Breast Cancer

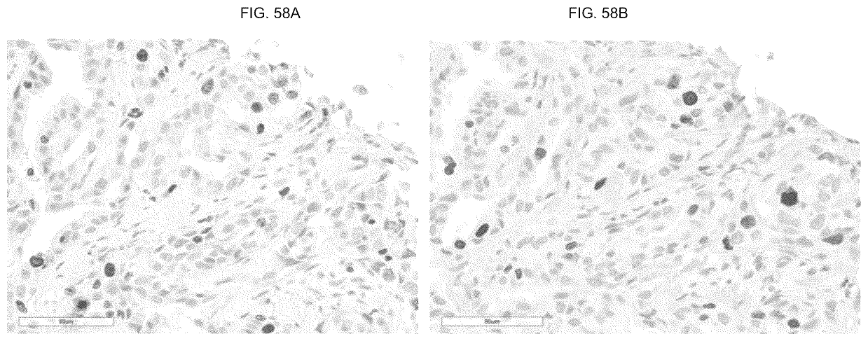

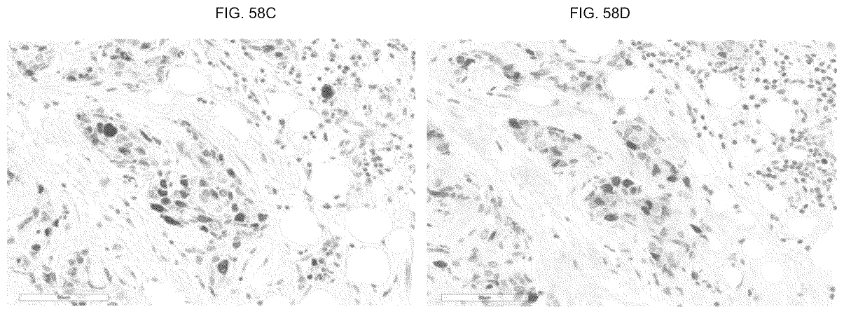

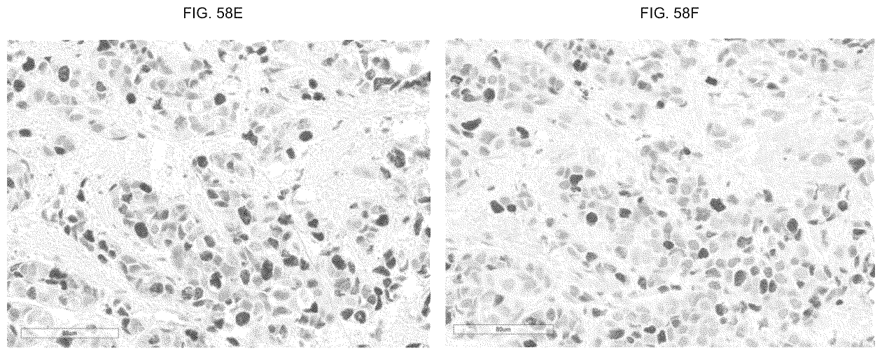

Prior to finalizing the investigational assay format, an informal comparison of the prototype with the LDT used in the Phase 2 neoadjuvant study of abemaciclib and anastrozole in HR+, HER2−breast cancer was performed. A similar quality of immunoreactivity was observed between both assays (FIG. 58A-F). In some cases, minor differences were observed in hematoxylin appearance and nuclear morphology. When the same scoring method was applied to both assays, a high degree of concordance was observed across the small sample set (see Table 7, below).

### Ki-67 IHC Assay Specificity

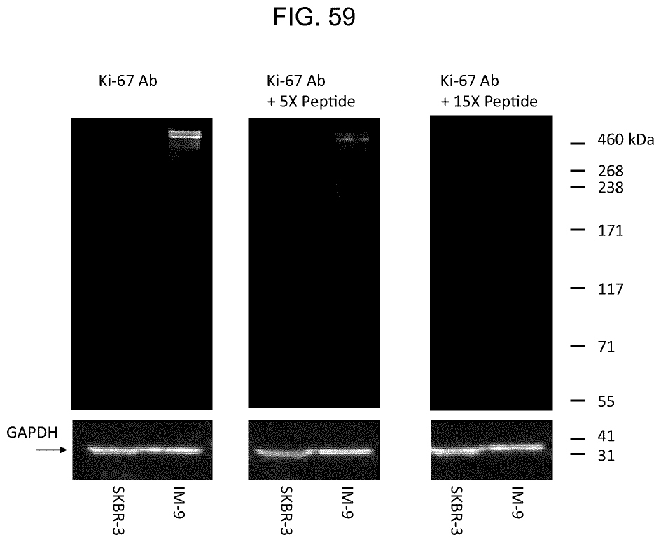

The specificity of the MIB-1 clone for the Ki-67 antigen has been described previously. This study confirmed that MIB-1 detected protein bands corresponding to the expected sizes for Ki-67 antigen of 345 kDa and 395 kDa on a Western blot using lysates of the cell line IM-9. As expected, cell line SKBR-3 did not express detectable Ki-67 protein as the level of Ki-67 RNA expressed is minimal (FIG. 59). The binding of Ki-67 MIB-1 antibody to the Ki-67 protein could be completely abrogated by the addition of a peptide containing the region reported to contain the epitope of this antibody (FIG. 59). When MIB-1 antibody was preincubated with five times excess by weight of the peptide, partial blocking of the binding was achieved and when the peptide was added at fifteen 15 times excess by weight, no binding was detected.

Table 4, below, summarizes the IHC-based specificity testing on 31 normal tissues demonstrating that Ki-67 IHC detects Ki-67 protein in the appropriate tissue elements and cellular compartments. Nuclear staining was observed in the majority of specimens and there were no unexpected results observed in cell types or tissue types tested. The observed staining was consistent with the reported literature for Ki-67 IHC expression in normal tissues.

### Ki-67 IHC Assay Sensitivity

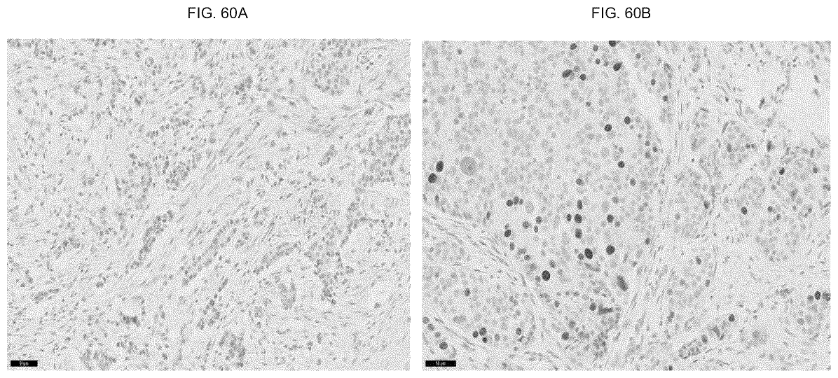

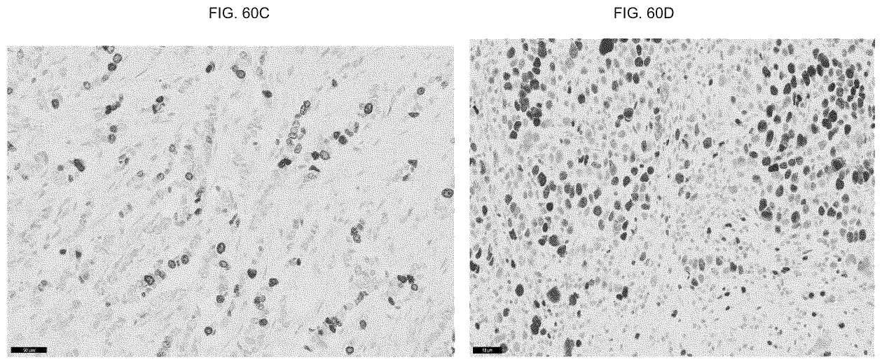

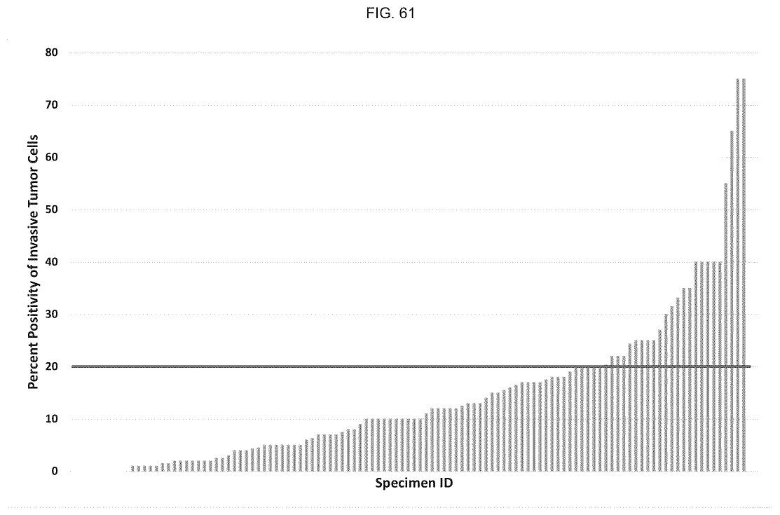

The prevalence of Ki-67 protein was evaluated in 113 unique FFPE specimens stained for Ki-67 with the clone MIB-1 monoclonal antibody, irrespective to HR or HER2 status. Specimens reflected a range of Ki-67 expression levels (FIG. 60A-D, FIG. 61) and included both resection (n=80) and CNB (n=33) specimens. Ki-67 expression was observed in tumor cells and a subset of benign elements including lymphocytes, stromal cells, and epithelium. Ki-67 IHC consistently detected Ki-67 protein across a relevant expression range (0 to 75%) in commercially procured specimens (FIG. 61). Seventy-four percent of the specimens were negative based on the cut-off (less than (<) 20% tumor cells positive) and 26% were positive (greater than (>) 20% tumor cells positive). This prevalence is consistent with reported literature. The distribution was similar in resection specimens and in CNB specimens (data not shown).

### Ki-67 IHC Assay Robustness

Robustness testing was conducted to evaluate the staining performance of the Ki-67 IHC assay under various laboratory conditions. The Ki-67 IHC assay demonstrated compatibility with Dako OMNIS™ workflow options that allow the instrument to be pre-programmed and pre-loaded for a delayed start and used overnight or over a weekend, with slides being removed from the loading tray when the staining is complete. In addition, the Ki-67 IHC assay achieved highly consistent results when tested with a range of target retrieval solution pH values between 5.9 and 6.5 and tissue section thicknesses between 3 to 6 μm. Analysis results are summarized in Table 5. Evaluation of pre-analytic variables demonstrated that an ischemic time of 1 hour or less is tolerated and fixation with 10% NBF is required, which is consistent with the optimal testing recommendations for estrogen receptor (ER), progesterone receptor (PR) and HER2 biomarker testing in breast cancer. Tonsil specimens fixed with acetic formalin alcohol, 10% unbuffered formal and Bouin's solution, or tonsil held in 70% ethanol for 4 to 5 days following fixation in 10% NBF, demonstrated altered immunostaining intensity in Ki-67 positive cells when compared to the reference condition, 10% NBF for 24 hours. Fixation times between 6 to 72 hours produced equivalent results, when 10% NBF was used and ischemia times were kept at or below 1 hour.

Tumor Heterogeneity Assessed with the Exemplary Ki-67 IHC Assay

Intra-block and intra-case heterogeneity studies showed high agreement rates (Table 5). Results demonstrate that Ki-67 diagnostic classification was consistent within tissue blocks and between sister blocks from the same case.

### Ki-67 IHC Assay Precision Studies

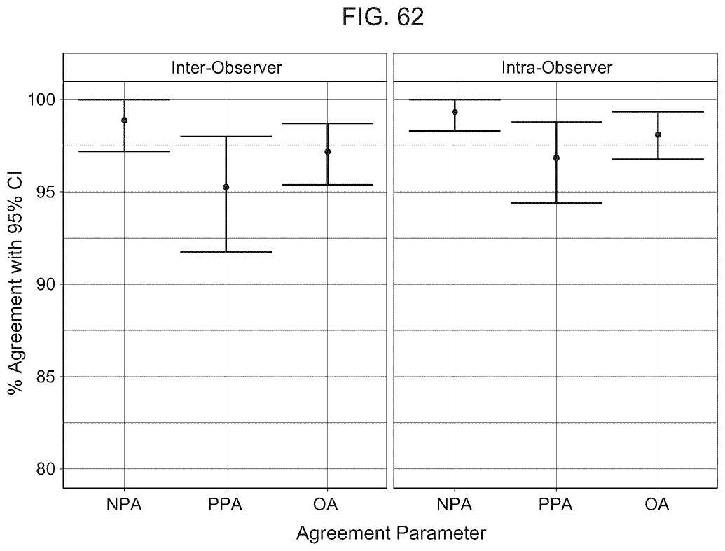

All precision studies achieved 95% lower bound confidence interval (LBCI) of greater than (>) 90% for NPA, PPA, and OA. Analysis results for internal studies are summarized in Table 7. The results for internal inter- and intra-observer studies are illustrated in FIG. 62. In three instances a specimen was determined to have fewer than the 200 viable tumor cells required for evaluation and therefore only 537 observations were included in the analysis. Inter-observer reproducibility testing achieved NPA, PPA, and OA point estimates of 98.9%, 95.2%, and 97.2%, respectively, with 2-sided 95% LBCI values of 97.2%, 91.7%, and 95.4%, respectively. For intra-observer reproducibility, NPA, PPA, and OA point estimates of 99.3%, 96.8%, and 98.1%, respectively, were achieved, with 95% LBCI values of 98.3%, 94.4%, and 96.8%, respectively.

Additional exploratory scoring was performed on a subset of early studies prior to assay validation to assess the potential utility of a hot-spot scoring approach. Hot-spot scores tended to produce larger average standard deviations across low and high Ki-67 expressing tumors and resulted in lower concordance among observers (data not shown), which could in part be explained by less stringent observer training for this method. These findings are congruent with previous observations of similar scoring comparisons.²⁵

### External Reproducibility of the Exemplary Ki-67 IHC Assay Across Multiple Sites

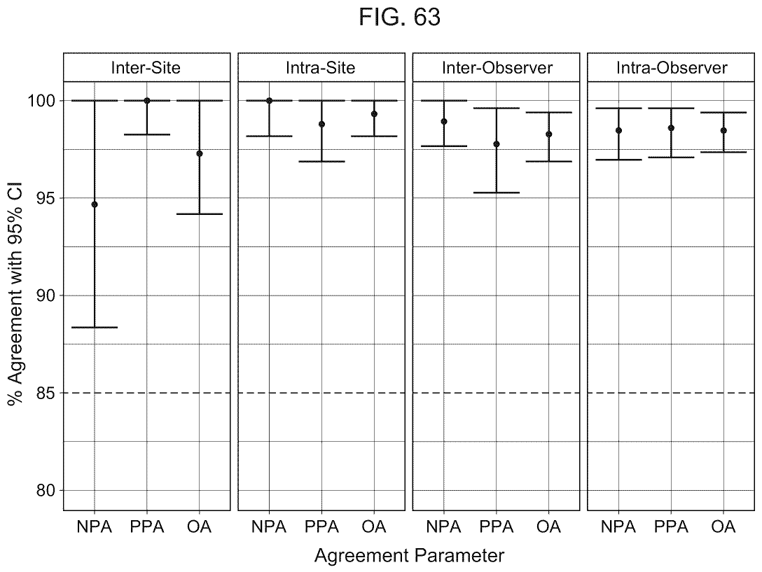

Inter- and intra-site reproducibility was performed by testing staining and scoring reproducibility across and within 3 external sites on a total of 15 replicates of unstained slide sets (n=30). The analyses were performed on 450 observations (comparisons to consensus), and the consensus was determined as the majority call for the sample across all 15 observations and across 5 observations for the sample-site combination. For inter-site reproducibility, NPA, PPA, and OA point estimates of 94.7%, 100.0%, and 97.3%, respectively, were achieved, with 2-sided 95% LBCI values of 88.4%, 98.3%, and 94.2%, respectively (FIG. 63). Intra-site reproducibility was evaluated by testing the staining and scoring reproducibility within a site across each of the 5 testing runs. The consensus for intra-site reproducibility analysis was determined as the majority call for the sample across all 5 observations within a given site. For intra-site reproducibility, NPA, PPA, and OA point estimates of 100.0%, 98.8%, and 99.3%, respectively, were achieved, with 95% LBCI of 98.2%, 96.9%, and 98.2%, respectively (FIG. 63). Since all inter- and intra-site reproducibility study parameters met pre-defined acceptance criteria, this study demonstrates that the Ki-67 IHC assay is reproducible across multiple sites as well as within the same site over multiple days/runs.

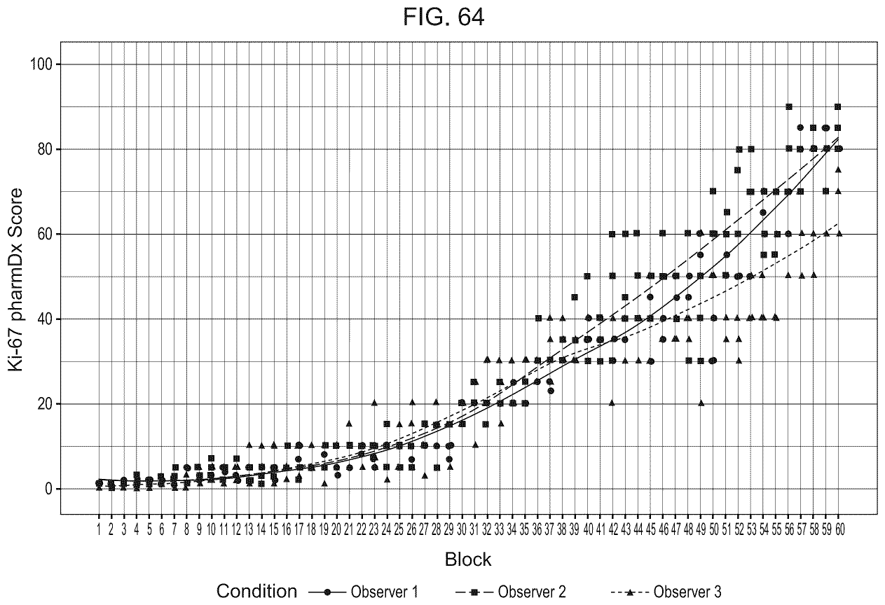

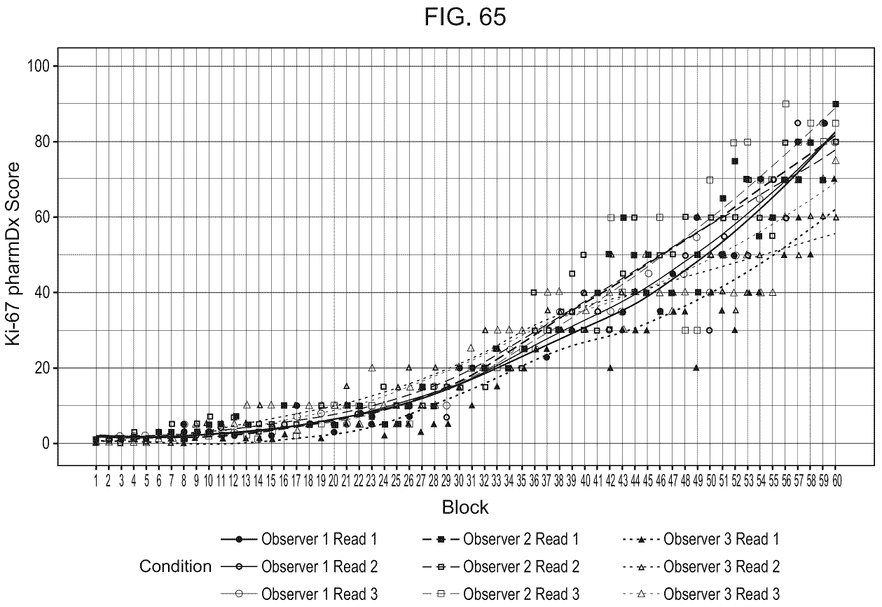

Inter-observer precision was also evaluated externally by testing scoring reproducibility between 3 trained and certified pathologists from different laboratories, who each performed 3 independent Ki-67 evaluations on a set of 60 pre-stained specimens. Therefore, the analyses were performed on 540 observations and the consensus was the majority call for the sample across all 9 observations. For inter-observer reproducibility, NPA, PPA, and OA point estimates of 98.9%, 97.8%, and 98.3%, respectively, were achieved, with 2-sided 95% LBCI values of 97.7%, 95.3%, and 96.9%, respectively (FIG. 63). Intra-observer precision was evaluated by testing scoring reproducibility within each of the 3 external pathologists using the same set of scores from the 60 specimens over 3 blinded and randomized reads. The analyses were performed on 540 observations and the consensus was the majority call across 3 observations for the sample-observer combination. For intra-observer reproducibility, NPA, PPA, and OA point estimates of 98.5%, 98.6%, and 98.5%, respectively, were achieved, with 95% LBCI values of 97.0%, 97.1%, and 97.4%, respectively (FIG. 63). Observers followed similar trends in scoring across the dynamic range. As expected, the variability in scores does increase as the Ki-67 expression range increases. However, there is not marked inter-observer variability (FIG. 64 and FIG. 65). These results demonstrate that the greater than or equal to (≥) 20% cut-off is reproducible within and between pathologists from different laboratories, when used on breast carcinoma specimens stained with the Ki-67 IHC and scored with the Ki-67 IHC algorithm.

### DISCUSSION

Ki-67 determined by IHC is a familiar biomarker and widely used for assessment of tumor proliferation in several tumor types; however, broad clinical adoption of its use in the management of breast carcinoma has been limited by lack of standardization. Despite some oncologists considering Ki-67 IHC in making treatment decisions for breast carcinoma, the American Society of Clinical Oncology has not endorsed the use of Ki-67 to determine whether a patient should receive chemotherapy or to guide adjuvant endocrine therapy selection. The International Ki67 in Breast Cancer Working Group performed several international ring trials to determine inter- and intra-laboratory reproducibility and identify key sources of variation (Phase 1) and to determine whether standardization of Ki-67 scoring methodology could result in high concordance (Phase 2). Based on the findings of both studies the report stated that “unless and until preanalytical and analytical features for IHC of Ki67 can be standardized, this assay platform should not be used to drive patient-care decisions in clinical practice”. A Phase 3 ring trial performed on core-cut biopsies reported higher levels of inter-observer concordance, but despite progress in standardization of the assay, additional investigation was warranted before Ki-67 IHC can be recommended for driving patient-care decisions.

The first prototype antibody for Ki-67 which recognized a human nuclear antigen associated with cell proliferation in a Hodgkin lymphoma cell line was discovered in 1983. However, the prototype Ki-67 antibody could only be used on frozen sections, and several monoclonal and polyclonal antibodies against the Ki-67 antigen were subsequently developed. A study comparing Ki-67 equivalent antibody clones (MIB-1, MM1, NCL-Ki-67p, and Rah Ki-67) reported that the MIB-1 antibody clone had higher sensitivity and better visual staining, with more diffusely and strongly stained nuclei, compared to the other clones. Furthermore, the International Ki67 in Breast Cancer Working Group has recommended MIB-1 as a “gold standard” for proliferation analysis.

Therefore, the exemplary Ki-67 THC assay was developed utilizing the MTB-1 clone, which was produced in a manufacturing facility at Dako North America under stringent quality control conditions appropriate for a US class III in vitro diagnostic device. Staining was performed on FFPE tissue, which provides greater convenience compared to using frozen tissue for use of the assay in pathological examinations and for precision medicine. Analytical studies were performed on resection and core needle biopsy specimens, which closely reproduces how breast carcinoma is managed in clinical practice. The Ki-67 IHC assay consistently detected the Ki-67 antigen across a range of Ki-67-expressing commercially procured FFPE breast carcinoma samples (expression level of 0 to 75%). Specificity data demonstrated the MIB-1 clone is specific for the Ki-67 antigen and that the assay detected the protein in appropriate tissue elements and cellular compartments in both normal and neoplastic specimens.

The international recommendations for Ki-67 assessment has proposed a minimum of 500 malignant invasive cells counted as well as including data from hotspots in the overall score. Denkert et al. reported that when using a 15% cut-off value for Ki-67, counting at least 500 to 1000 cells is necessary to achieve an acceptable error rate. To maximize available clinical trial specimens while maintaining a high degree of scoring reproducibility, the scoring algorithm and guidelines were developed with a minimum requirement of 200 viable invasive tumor cells that must be present in the specimen to determine the percentage of Ki-67 positive cells, although most specimens tended to contain tumor content well above the minimum. The scoring methodology proved to be robust and consistent, even when non-serial sections were used. Several studies have reported challenges in Ki-67 IHC interpretation that may be due to intra-tumoral heterogeneity issues including spatial and temporal heterogeneity issues associated with Ki-67. Heterogeneity can lead to substantial differences in Ki-67 scores between individual samples of the same specimen. In this study, intra-block and intra-case (sister block) heterogeneity studies demonstrated high overall percent agreement, with a point estimate of 96.5% and a LBCI of 92.4% and a point estimate of 96.0% with a LBCI of 90.0%, respectively.

In addition to developing standardized guidelines for Ki-67 scoring, systemic training prior to conduct of studies was incorporated. Internal observers and external laboratory pathologists were trained and tested on the scoring algorithm/guidelines. In both the Phase 2 and Phase 3 international ring trials, evaluating pathologists were able to achieve better concordance in Ki-67 assessment when the laboratories followed standardized training and scoring methods. In this study, we report the external validation and comparability of Ki-67 staining and scoring both within and between sites following pathologist training. The external reproducibility study results demonstrated robust reproducibility of scoring with the Ki-67 IHC assay in breast carcinoma specimens with point estimates of 98.3% (inter-observer reproducibility) and 98.5% (intra-observer reproducibility).

The Ki-67 IHC assay was designed to be run on the Dako OMNIS™ platform which employs an easy user interface, fully automated workflow to ensure consistency of staining results, continuous patient case delivery, and allows for fast turn-around time. Accessory reagent inter-lot testing was conducted to demonstrate that the reagents, used in combination with the Ki-67 IHC assay, produce consistent results in normal day-to-day testing. Repeatability within instruments, staining rack, and day as well as precision between Dako OMNIS™ instruments, testing days, and assay lots further confirmed that the assay produces consistent results in normal day-to-day testing. Robustness studies (target retrieval solution pH, tissue section thickness, and overnight/over weekend staining) demonstrated that the Ki-67 IHC assay produces consistent Ki-67 staining under various laboratory conditions.

While the studies have shown high reproducibility in scoring across laboratories, there may be opportunities for further improvement in standardization of the assay. Limitations may exist with manual interpretation of Ki-67 scoring using a qualitative positive/negative outcome based upon a pre-defined cut-off. Emergence of digital image analysis (DIA) platforms may mitigate some of these potential limitations. In a study by Acs et al., the authors investigated the reproducibility of Ki-67 measurement between three DIA platforms and found high inter- and intra-DIA platform reproducibility. Another limitation of this study is that the Ki-67 IHC pharmDx has so far only been validated on 1 staining platform (Dako OMNIS™). Furthermore, prior to conducting these validation studies, there was no generally accepted scoring method that had utility of Ki-67 as a selective biomarker in a randomized clinical study. Scoring of the entire slide was chosen over a hot spot scoring method, since additional variability, potentially due to different scorers identifying different areas as hot spots, was observed when using the hot spot method. An additional concern was that samples that do not present with an obvious hot spot may be rendered unevaluable without further guidance. Acceptance of a standardized Ki-67 assay interpretation guideline would possibly enable the refinement of clinical society guidelines and recommendations for Ki-67 assays in the future.

In summary, we describe the development and analytical validation of an Ki-67 IHC assay for risk determination in breast carcinoma that is sensitive, specific, precise, robust, and reproducible across different laboratories is described. This novel assay has been standardized to ensure that Ki-67 results are reproducible and therefore clinically relevant. This Ki-67 IHC assay was used in a Phase 3 clinical trial of abemaciclib combined with standard adjuvant endocrine therapy versus standard adjuvant endocrine therapy alone in patients with early, HR+, HER2−breast cancer. The assay performed on FFPE tissue, using an automated platform, has well-established analytical performance and therefore may allow for broader worldwide implementation to help select breast cancer patients where Ki-67 expression is relevant. We have shown that standardization of the Ki-67 scoring methodology, as well as the pre-analytical and analytical variables, resulted in high concordance in staining and scoring across multiple laboratories. These advancements represent progress towards testing the utility of Ki-67 THC to enable patient-care decisions in the appropriate clinical setting.

### Figure Legends

FIG. 57. Breast carcinoma specimen stained with Ki-67 primary antibody exhibiting both negative and weak positive staining. Negative cells show grey hematoxylin counterstaining and are indicated with black arrows. Cells with weak positive 1+ staining are indicated with green arrows (20× objective; scale bar is 50 μm).

FIG. 58: Qualitative comparison of the prototype Ki-67 assay with the neoadjuvant study of abemaciclib and anastrozole in HR+, HER2−breast cancer LDT. Representative images were captured from tumors with a Ki-67 positivity near the greater than or equal to (≥) 20% cut-off. Breast carcinoma tissue sections in the left column were assayed with the prototype assay (A, 12%; C, 25%; E, 35%); nearby tissue levels were assayed with the LDT as shown on the right (B, 10%; D, 15%; F, 35%). Respective scores correspond to the entire specimen. Images A-F are representative fields captured using a 20× objective; scale bar is 80 μm. Abbreviations: LDT, laboratory developed test.

FIG. 59: Ki-67 protein is expressed in IM-9 cells, a cell line from which the immunizing protein for MIB-1 was derived, and is not expressed in SKBR-3 cells, a cell line with a very low level of Ki-67 RNA expression. A peptide comprising a region within the immunizing protein when preincubated with the antibody reduces the binding to the IM-9 protein. The GAPDH loading control demonstrates equal loading of the cell lysates. Abbreviations: GAPDH, glyceraldehyde-3-phosphate dehydrogenase.

FIG. 60: Expression levels in breast carcinoma tissue bank specimens stained with the Ki-67 assay. Ki-67 was detected over a relevant range of in breast cancer FFPE specimens. Images shown are from archived tumor samples with a Ki-67 score of 0% (A), a Ki-67 score of 19% (B), a Ki-67 score of 28% (C), and a Ki-67 score of 52% (D), respectively (20× objective; scale bar is 50 μm). Abbreviations: FFPE, formalin-fixed, paraffin-embedded.

FIG. 61: Ki-67 IHC Assay Sensitivity. Dynamic range distribution of scores across 113 breast cancer samples, including resections and core needle biopsies. The green line indicates the diagnostic cut-off greater than or equal to (≥) 20%.

FIG. 62: Summary of percent agreement for Ki-67 IHC assay observer precision studies performed internally at Dako North America.

FIG. 63: Summary of percent agreement for Ki-67 IHC assay external reproducibility studies performed at three external sites. Left 2 graphs demonstrate inter- and intra-site reproducibility which measures assay staining and scoring interpretation. Right 2 graphs demonstrate inter- and intra-observer reproducibility which measures scoring interpretation only. Horizontal dashed lines indicate acceptance criteria for the external reproducibility studies.

FIG. 64: Locally estimated scatterplot smoothing (LOESS) plot of external reproducibility inter-observer Ki-67 continuous scores grouped by observer. LOESS lines demonstrate average trends over inter-observer data using locally weighted regression. Abbreviations: LOESS, locally estimated scatterplot smoothing.

FIG. 65: Locally estimated scatterplot smoothing (LOESS) plot of external reproducibility inter-observer Ki-67 continuous scores grouped by observer/read combination. LOESS lines demonstrate average trends over inter-observer data using locally weighted regression.

Abbreviations: LOESS, locally estimated scatterplot smoothing.

TABLE 6
Summary of

| pre-analytic, analytic | | | |
|---|---|---|---|
| and post-analytic | | | |
| factors considered in | | | |
| development of the | | Ki-67 IHC Assay | |
| Ki-67 IHC Assay | Factor | Overview | |
| Preanalytical | Biopsy type | Core biopsy or surgical | |
| | | resection specimens | |
| | Time to fixation | Less than or equal to (≤) 1 | |
| | | hour | |
| | Fixation time and fixative | 6 to 72 hours in 10% | |
| | | neutral buffered formalin | |
| | Paraffin embedding | Tissues were infiltrated | |
| | | with melted paraffin, at or | |
| | | below 60° C. | |
| | Slide preparation | Tissue section of 4 to 5 | |
| | | μm were mounted on | |
| | | Dako FLEX IHC | |
| | | Microscope Slides or | |
| | | SuperFrost Plus slides | |
| | | Specimens were oven- | |
| | | dried at 58° C. +/−2° C. | |
| Analytical, ctd. | Detection system | Polymer based - | |
| | | EnVision FLEX | |
| | | Detection System (Code | |
| | | GV800) | |
| | Counterstain | EnVision FLEX | |
| | | Hematoxylin (Code | |
| | | GC808) | |
| | Quality control | Positive and negative | |
| | | control tissues were run | |
| | | for each staining | |
| | | procedure. Control tissues | |
| | | were fixed in the same | |
| | | way as the patient tissue. | |
| Post-analytical | Tissue specimen criteria | Invasive breast carcinoma | |
| (interpretation and | | Minimum 200 viable | |
| scoring) | | invasive tumor cells | |
| | | present in specimen | |
| Preanalytical, ctd. | Cut slide storage | Specimens were stained | |
| | | within 5 months of | |
| | | sectioning when stored in | |
| | | the dark at 2° C. to 8° C. or in | |
| | | the dark at room | |
| | | temperature up to 25° C. | |
| Analytical | Staining instrument | Dako OMNIS ™* | |
| | Antigen retrieval | EnVision FLEX Target | |
| | | Retrieval Solution, Low pH | |
| | | (Code GV805). pH of 1x | |
| | | solution 6.1 +/− 0.2. | |
| | | Antigen retrieval took | |
| | | place onboard the Dako | |
| | | OMNIS ™ instrument at | |
| | | 97° C. | |
| | Specific antibody | Purified monoclonal mouse | |
| | | anti-Ki67, clone MIB-1 | |
| | | (Code GE020) | |
| | Negative Control | Protein matched, mouse | |
| | Reagent | immunoglobulin G isotype | |
| | | control. Negative Control | |
| | | Reagent (Code GE020) | |
| Post-analytical | Tissue specimen criteria, | Only well-preserved and | |
| (interpretation and | ctd. | well-stained areas of the | |
| scoring), ctd. | | specimen were used to | |
| | | determine the percentage of | |
| | | positive tumor cells | |
| | | Only the invasive cancer | |
| | | component was scored; in | |
| | | situ carcinoma was not | |
| | | scored. | |
| | Whole tissue score | The entire tissue section | |
| | methodology | was scored. If hotspots were | |
| | | present in the section, they | |
| | | were an integral part of the | |
| | | final score. For whole tissue | |
| | | evaluation, objectives of | |
| | | 10-20× magnification are | |
| | | appropriate. Only nuclear | |
| | | staining of tumor cells was | |
| | | scored. | |
| Post-analytical | Whole tissue score | The percentage of positively | |
| (interpretation and | methodology, ctd. | stained tumor cells among the total | |
| scoring), ctd. | | number of invasive cells across the | |
| | | entire slide was determined. | |
| | | Nuclear staining at all intensities | |
| | | (1-3+) was included. The lower | |
| | | limit of 1+ positivity was evaluated | |
| | | using a high-power (e.g., 20×) | |
| | | objective and defined by the | |
| | | following rules: | |
| | | Signal must be unequivocally | |
| | | brown | |
| | | The staining must correspond to | |
| | | a nucleus | |
| | | The staining must cover the | |
| | | whole chromatin distribution | |
| | | within the nucleus | |
| | | The staining must correspond to | |
| | | a non-apoptotic cell | |
| Post-analytical | Cut-off | Positive | Negative |
| (interpretation and | | Greater than or | Less than (<) 20% |
| scoring), ctd. | | equal to (≥) 20% | of invasive tumor |
| | | of invasive tumor | cells stained |
| | | cells stained | |

Abbreviations: IHC, immunohistochemistry.
*Fully automated staining, from deparaffinization to counterstaining

TABLE 7
Summary of Ki-67 IHC Assay normal tissue reactivity

| Tissue Type (# tested) | Positive Cell Staining: Tissue Elements | Non-specific Staining |
|---|---|---|
| Adrenal (3) | 2/3 Scattered adrenal cortex cells | 0/3 |
| Bone marrow (3) | 2/3 Marrow cells | 0/3 |
| Breast (3) | 2/3 subset ductal epithelial cells* | 0/3 |
| | 2/3 rare myoepithelial cells* | |
| Cerebellum (3) | 0/3 | 0/3 |
| Cerebrum (3) | 1/3 Rare oligodendrocytes | 0/3 |
| Cervix (3) | 2/3 Rare epithelial cells | 0/3 |
| | 1/3 parabasal squamous cells | |
| Colon (3) | 3/3 Crypt epithelial cells | 0/3 |
| Esophagus (3) | 3/3 Parabasal squamous cells | 0/3 |
| Kidney (3) | 3/3 Rare tubule cells | 0/3 |
| Liver (3) | 3/3 Rare hepatocytes | 0/3 |
| Lung (3) | 3/3 Rare type 1 alveolar cells | 0/3 |
| | 2/3 Pulmonary macrophages | |
| Mesothelial cells (3) | 1/3 Rare mesothelial cells | 0/3 |
| Muscle, cardiac (3) | 1/3 Few cardiac myocytes | 0/3 |
| Muscle, skeletal (3) | 0/3 | 0/3 |
| Nerve, peripheral (3) | 0/3 | 0/3 |
| Ovary (3) | 2/3 Follicle cyst lining cells | 0/3 |
| | 1/3 Rare ovarian stromal cells | |
| Pancreas (3) | 3/3 Rare acinar cells | 0/3 |
| Parathyroid (3) | 3/3 Rare endocrine cells/Chief cells | 0/3 |
| Pituitary (3) | 1/3 Lymphocytes | 0/3 |
| | 2/3 Pituicytes | |
| Prostate (3) | 3/3 Rare epithelial cells and stromal cells | 0/2 |
| Salivary gland (3) | 1/3 Rare acinar epithelial cells | 0/3 |
| Skin (3) | 3/3 Suprabasal squamous cells | 0/3 |
| Small intestine (3) | 2/3 Crypt epithelium | 0/3 |
| Spleen (3) | 3/3 Few red pulp cells | 0/3 |
| | 1/3 white pulp cells | |
| Stomach (3) | 3/3 Gastric epithelial cells | 0/3 |
| Testis (3) | 3/3 Spermatogonia | 0/3 |
| Thymus (3) | 3/3 Thymic cortex | 0/3 |
| Thyroid (3) | 0/3 | 2/3 Scattered interspersed inflammatory cells |
| Tonsil (3) | 3/3 Squamous parabasal cells | 0/3 |
| | 3/3 Germinal centers | |
| | 3/3 interfollicular cells | |
| Urinary bladder (3) | 3/3 Urothelial cells | 2/3 Interspersed inflammatory cells |
| Uterus (3) | 3/3 Endometrial epithelium and stroma | 0/3 |

*May serve as an internal tissue control element. Abbreviations: IHC, immunohistochemistry.

TABLE 8
Agreements and 95% confidence intervals of Ki-67 IHC Assay robustness
and heterogeneity studies performed at Dako North America

| | Number of | |
|---|---|---|
| | Comparisons | |
| Study | (Total) | % Agreement (95% CI) |
| Overnight/over weekend | 270 | NPA 99.3% (97.8-100.0%) |
| (delayed start) | | PPA 95.6% (90.4-100.0%) |
| | | OA 97.4% (94.4-99.6%) |
| 1x Target Retrieval | 384 | NPA 100% (98.4-100.0%) |
| Solution pH | | PPA 97.9% (94.4-100.0%) |
| (pH 5.9-6.5) | | OA 99.2% (97.9-100.0%) |
| Tissue section thickness | 384 | NPA 98.8% (96.7-100.0%) |
| (3 μm-6 μm) | | PPA 97.2% (91.7-100.0%) |
| | | OA 98.2% (95.6-100.0%) |
| Intra-block heterogeneity | 144 | NPA 94.0% (86.9-100.0%) |
| | | PPA 100% (94.0-100.0%) |
| | | OA 96.5% (92.4-100.0%) |
| Intra-case heterogeneity | 50 | NPA 94.1% (85.3-100.0%) |
| | | PPA 100% (80.6-100.0%) |
| | | OA 96.0% (90.0-100.0%) |

Abbreviations: CI, confidence interval; NPA, negative percent agreement; OA, overall percent agreement; PPA, positive percent agreement.

TABLE 9
Agreements and 95% confidence intervals of Ki-67 IHC Assay
precision studies performed at Dako North America

| | Number of | |
|---|---|---|
| | Comparisons | |
| Internal Repeatability | (Total) | % Agreement (95% CI) |
| Inter-day | 480 | NPA 98.3% (96.2-100.0%) |
| | | PPA 99.2% (97.5-100.0%) |
| | | OA 98.8% (97.5-99.8%) |
| Inter-instrument | 480 | NPA 98.8% (96.7-100.0%) |
| | | PPA 97.5% (94.6-100.0%) |
| | | OA 98.1% (96.5-99.6%) |
| Inter-lot (Ki-67 IHC) | 360 | NPA 98.9% (97.2-100.0%) |
| | | PPA 97.2% (92.8-100.0%) |
| | | OA 98.1% (95.8-99.7%) |
| Inter-lot (accessory | 192 | NPA 95.1% (88.2-100.0%) |
| reagents) | | PPA 100% (95.9-100.0%) |
| | | OA 97.4% (93.8-100.0%) |
| Intra-instrument/rack/day | 480 | NPA 98.8% (96.7-100.0%) |
| | | PPA 97.5% (94.6-100.0%) |
| | | OA 98.1% (96.2-99.6%) |

Abbreviations: CI, confidence interval; NPA, negative percent agreement; OA, overall percent agreement; PPA, positive percent agreement.

TABLE 10
Concordance between the prototype assay and the LDT neoadjuvant
study of abemaciclib and anastrozole in HR+, HER2−
breast cancer when assessed with the same scoring method
by a single observer. Note: Seven cores were not evaluable.

| | | LDT | | |
|---|---|---|---|---|
| | | Positive | Negative | Total |
| Ki-67 IHC | Positive | 24 | 4 | 28 |
| Prototype | Negative | 0 | 32 | 32 |
| | Total | 24 | 36 | 60 |

Abbreviations: IHC, immunohistochemistry; LDT, laboratory developed test.

### REFERENCES

1. Lindboe C F, Torp S H. Comparison of Ki-67 equivalent antibodies. J Clin Pathol. 2002; 55(6):467-471.
2. Luporsi E, Andre F, Spyratos F, et al. Ki-67: level of evidence and methodological considerations for its role in the clinical management of breast cancer: analytical and critical review. Breast Cancer Res Treat. 2012:132(3):895-915.
3. Urruticoechea A, Smith I E, Dowsett M. Proliferation marker Ki-67 in early breast cancer. J Clin Oncol. 2005; 23(28):7212-7220.
4. Stuart-Harris R, Caldas C, Pinder S E, Pharoah P. Proliferation markers and survival in early breast cancer a systematic review and meta-analysis of 85 studies in 32,825 patients. Breast. 2008:17(4):323-334.
5. Fasching P A, Gass P, Haberle L, et al. Prognostic effect of Ki-67 in common clinical subgroups of patients with HER2-negative, hormone receptor-positive early breast cancer. Breast Cancer Res Treat. 2019; 175(3):617-625. 620 Bustreo S, Osella-Abate S, Cassoni P, et al. Optimal Ki67 cut-off for luminal breast cancer prognostic evaluation: a large case series study with a long-term follow-up. Breast Cancer Res Treat. 2016; 157(2):363-371.
7. Tashima R, Nishimura R, Osako T, et al. Evaluation of an optimal cut-off point for the Ki-67 index as a prognostic factor in primary breast cancer: a retrospective study. PLoS One. 2015:10 (7):e0119565.
8. Niazi M K K, Downs-Kelly E, Gurcan M. Hot spot detection for breast cancer in Ki-67 stained slides: image dependent filtering approach. Vol 9041: SPIE; 2014.
9. Inwald E C, Klinkhammer-Schalke M, Hofstadter F, et al. Ki-67 is a prognostic parameter in breast cancer patients: results of a large population-based cohort of a cancer registry. Breast Cancer Res Treat. 2013; 139(2):539-552.
10. Dowsett M, Nielsen T O, A'Hern R, et al. Assessment of Ki67 in breast cancer-recommendations from the International Ki67 in Breast Cancer working group. JNail Cancer Inst. 2011; 103(22):1656-1664.
11. Sledge G W, Jr., Toi M, Neven P, et al. MONARCH 2: abemaciclib in combination with fulvestrant in women with HR+, HER2−advanced breast cancer who had progressed while receiving endocrine therapy. J Clin Oncol. 2017; 35(25):2875-2884. 120 Dickler M N, Tolaney S M, Rugo H S, et al. MONARCH 1, a phase I I study of abemaciclib, a CDK4 and CDK6 Inhibitor, as a single agent, in patients with refractory H R(+)/HER2(−) metastatic breast cancer. Clin Cancer Res. 2017:23(17):5218-5224.
13. Johnston S, Martin M, Di Leo A, et al. MONARCH 3 final PFS: a randomized study of abemaciclib as initial therapy for advanced breast cancer. NPJBreast Cancer. 2019; 5:5.
14. Hurvitz S A, Martin M, Press M F, et al. Potent cell-cycle inhibition and upregulation of immune response with abemaciclib and anastrozole in neoMONARCH, phase 11 neoadjuvant study in H R(+)/HER2(−) breast cancer. Clin Cancer Res. 2020:26(3):566-580.
15. Lilly E, Company, Inc N F. Endocrine Therapy With or Without Abemaciclib (LY2835219) Following Surgery in Participants With Breast Cancer. https://ClinicalTrials.gov/show/NCT03155997; 2017.
16. FDA. Guidance for Submission of Tmmunohistochemistry Applications to the FDA 1998.
17. Key G, Becker M H, Baron B, et al. New Ki-67-equivalent murine monoclonal antibodies (MIB 1-3) generated against bacterially expressed parts of the Ki-67 cDNA containing three 62 base pair repetitive elements encoding for the Ki-67 epitope. Lab Invest. 1993; 68(6):629-636.
180 Klijn C, Durinck S, Stawiski E W, et al. A comprehensive transcriptional portrait of human cancer cell lines. Nat Biotechnol. 2015; 33(3):306-312.
19. Cattoretti G, Becker M H, Key G, et al. Monoclonal antibodies against recombinant parts of the Ki-67 antigen (MIB 1 and MIB 3) detect proliferating cells in microwave-processed formalin-fixed paraffin sections. JPathol. 1992; 168(4):357-363.
20. Hsu C Y, Yang C F, Liao L R, Ho H L, Ho D M. Tonsil surface epithelium is ideal for monitoring Ki-67 immunohistochemical staining. Histopathology. 2013; 63(6):810-816.
21. Kurbel S, Dmitrovic B, Marjanovic K, Vrbanec D, Juretic A. Distribution of Ki-67 values within HER2 & E R/PgR expression variants of ductal breast cancers as a potential link between IHC features and breast cancer biology. BMC Cancer. 2017; 17(1):231.
22. Regan M M, Francis P A, Pagani 0, et al. Absolute benefit of adjuvant endocrine therapies for premenopausal women with hormone receptor-positive, human epidermal growth factor receptor 2-negative early breast cancer: TEXT and SOFT trials. J Clin Oncol. 2016; 34(19):2221-2231.
235 Allison K H, Hammond M E H, Dowsett M, et al. Estrogen and progesterone receptor testing in breast cancer American Society of Clinical Oncology/College of American Pathologists Guideline Update. Arch Pathol Lab Med. 2020; 144(5):545-563.
24. Wolff A C, Hammond M E H, Allison K H, et al. Human epidermal growth factor receptor 2 testing in breast cancer: American Society of Clinical Oncology/College of American Pathologists Clinical Practice Guideline Focused Update. Arch Pathol Lab Med. 2018; 142(11):1364-1382.
25. Leung S C Y, Nielsen T O, Zabaglo L, et al. Analytical validation of a standardized scoring protocol for Ki67: phase 3 of an international multicenter collaboration. NPJ Breast Cancer. 2016; 2:16014.
295 Harris L N, Ismaila N, McShane L M, Hayes D F. Use of biomarkers to guide decisions on adjuvant systemic therapy for women with early-stage invasive breast cancer: American Society of Clinical Oncology Clinical Practice Guideline Summary. J Oncol Pract. 2016; 12(4):384-389.
27. Polley M Y, Leung S C, McShane L M, et al. An international Ki67 reproducibility study. J Natl Cancer Inst. 18 2013; 105(24):1897-1906.
28. Polley M Y, Leung S C, Gao D, et al. An international study to increase concordance in Ki67 scoring. Mod Pathol. 2015; 28(6):778-786.
29. Gerdes J, Schwab U, Lemke H, Stein H. Production of a mouse monoclonal antibody reactive with a human nuclear antigen associated with cell proliferation. Int J Cancer. 1983; 31(1):13-20.
30. Denkert C, Budczies J, von Minckwitz G, Wienert S, Loibl S, Klauschen F. Strategies for developing Ki67 as a useful biomarker in breast cancer. Breast. 2015:24 Suppl 2:S67-72.
31. Tramm T, Kyndi M, Sorensen F B, Overgaard J, Alsner J. Influence of intra-tumoral heterogeneity on the evaluation of BCL2, E-cadherin, EGFR, EMMPRIN, and Ki-67 expression in tissue microarrays from breast cancer. Acta Oncol. 2018; 57(1):102-106.
320 Nassar A, Radhakrishnan A, Cabrero I A, Cotsonis G A, Cohen C. Intratumoral heterogeneity of immunohistochemical marker expression in breast carcinoma: a tissue microarray-based study. Appl Immunohistochem Mol Morphol. 2010; 18(5):433-441.
33. Acs B, Pelekanou V, Bai Y, et al. Ki67 reproducibility using digital image analysis: an inter-platform and inter-operator study. Lab Invest. 2019; 99(1):107-117.

A number of embodiments of the invention have been described. Nevertheless, it can be understood that various modifications may be made without departing from the spirit and scope of the invention. Accordingly, other embodiments are within the scope of the following claims.

What is claimed is:

1. A method for assessing the extent of Ki-67 expression in a tumor or a cancer comprising: contacting a tissue sample or a portion thereof from an individual having a tumor or a cancer with an antibody or portion thereof which specifically binds to Ki-67; and, determining a Ki-67 score (%) by dividing the number of Ki-67 staining viable tumor or cancer cells specifically bound by the antibody with the total number of staining and non-staining viable cancer or tumor cells and multiplying the result by 100, thereby obtaining the Ki-67 score (%).

2. An immunohistochemistry (IHC) method for determining and scoring the extent of nuclear expression of protein Ki-67 (also known as MK167) in a tissue sample, the method comprising:
(a) staining a tissue sample with an antibody which specifically binds to Ki-67; and
(b) determining the total number of viable invasive tumor or cancer cells having anti-Ki-67 nuclear staining, and determining the total number of staining and non-staining viable invasive tumor or cancer cells in at least a portion of the tissue sample,
wherein an invasive tumor or cancer cell is counted as positively stained with anti-Ki-67 if there is convincing and complete nuclear anti-Ki-67 staining, and if the invasive tumor or cancer cell displays at an anti-Ki-67 nuclear staining at any intensity above a defined threshold, and
(c) determining a Ki-67 score (%),
wherein the Ki-67 score (%) is the number of Ki-67 staining viable invasive tumor or cancer cells found in the tissue sample divided by the total number of staining and non-staining viable invasive tumor or cancer cells, multiplied by 100.

3. The method of claim 1 or claim 2, wherein the cancer or tumor cells are viable invasive breast carcinoma or breast cancer cells.

4. The method of any of claims 1 to 3, further comprising determining whether the Ki-67 score (%) is 10 or above; or, determining whether the Ki-67 score (%) is 20 or above.

5. The method of any of claims 1 to 4, or any of the preceding claims, wherein determining the number of Ki-67 positive viable tumor or cancer cells comprises determining the number of viable tumor or cancer cells: having Ki-67 staining in the nucleus, or, having Ki-67 staining throughout the entire chromatin distribution in the nucleus.

6. The method of any of claims 1 to 5, or any of the preceding claims, further comprising: determining whether a color reflecting binding of the antibody to Ki-67 is the intended color, wherein optionally the intended color is brown, or the brown color is generated by staining with 3,3′-Diaminobenzidine (DAB).

7. The method of any of claims 1 to 6, or any of the preceding claims, further comprising:
(a) excluding from the Ki-67 positive tumor or cancer cells at least one type of cell selected from the group consisting of: tumor cells or cancer cells with only cytoplasmic or membrane staining; non-invasive neoplasia or carcinoma in situ cells, non-viable or necrotic tumor or cancer cells, apoptotic nuclei or nuclear debris, tumor or cancer cells in poorly preserved tissue areas, benign epithelial cells, non-neoplastic cells and/or lymphocytes with nuclear staining, apoptotic cells, necrotic cells, cells not exhibiting an intended color, cells in which a stain reflecting of binding of the antibody to Ki-67 is not present throughout the entire chromatin distribution in the nucleus, cells exhibiting membrane staining, cells exhibiting cytoplasmic staining, lymphocytes, and stromal cells; or
(b) excluding from the portion of the tissue sample from which the Ki-67 score (%) is determined portions of the tissue sample exhibiting at least one artifact selected from the group consisting of distorted morphology, poor fixation, crush and cautery artifacts.

8. The method of any of claims 1 to 7, or any of the preceding claims, wherein:
(a) the Ki-67 score (%) is calculated in a portion of the tissue sample comprising at least 100 cells, or is calculated in a portion of the tissue sample comprising at least 200 cells; or
(b) the method further comprises administering a cancer therapeutic based on the Ki-67 score (%), and optionally the cancer therapeutic is an ATP competitive inhibitor of a cyclin-dependent kinase, or the ATP competitive inhibitor of a cyclin-dependent kinase is abemaciclib, or the cancer therapeutic comprises palbociclib or ribociclib.

9. The method of any of claims 1 to 8, or any of the preceding claims, wherein:
(a) the tissue sample is from a patient with node-positive, early, resected hormone receptor positive (HR+), human epidermal growth factor receptor 2 negative (HER2−) breast cancer; or
(b) the method further comprises determining that a subject is likely to respond favorably to treatment with a cancer therapeutic if the Ki-67 score (%) is above a threshold value, and optionally the threshold value is about 1%, about 5%, about 10%, about 20%, about 30%, about 40%, about 50% or about any number between 1% and 50%; or
(c) the cancer therapeutic is a cyclin dependent kinase inhibitor, and optionally the cyclin dependent kinase inhibitor is a CDK4 or CDK6 inhibitor, or the cyclin dependent kinase inhibitor is abemaciclib, palbociclib or ribociclib; or
(d) the cancer is a breast cancer or breast carcinoma, a head and neck cancer, a colorectal cancer, a bladder cancer, a lung cancer, gastrointestinal stromal tumors (GIST), prostate cancer, cervical cancer, or renal cell carcinoma, or the cancer is metastatic breast cancer; or
(e) the method further comprises administering the cancer therapeutic if the Ki-67 score (%) is above the threshold value; or
(f) the method further comprises administering a treatment other than a cyclin dependent kinase inhibitor if the Ki-67 score (%) is below the threshold value.

10. The method of any of claims 1 to 9, or any of the preceding claims, wherein it is determined if the tissue section is or is not adequate for the determining and scoring of the amount of nuclear expression of protein Ki-67, and a tissue section is considered adequate for evaluation if about 100 or more Ki-67 stained viable invasive tumor or cancer cells are present.

11. The method of any of claims 1 to 10, or any of the preceding claims, wherein the Ki-67 score (%) is the number of Ki-67 staining viable invasive tumor or cancer cells found in the tissue sample divided by the total number of staining and non-staining viable invasive tumor or cancer cells, multiplied by 100.

12. The method of any of claims 1 to 11, or any of the preceding claims, wherein an invasive tumor or cancer cell is counted as positively stained with anti-Ki-67 if there is convincing and complete nuclear anti-Ki-67 staining, and if the invasive tumor or cancer cell displays at an anti-Ki-67 nuclear staining at any intensity of 1+ or higher.

13. The method of any of claims 1 to 12, or any of the preceding claims, wherein:
(a) a section or portion of the tissue sample is prepared on a slide or equivalent, and the section or portion of the tissue sample is stained on the slide; or
(b) the antibody which specifically binds to Ki-67 comprises a monoclonal mouse anti-Ki-67 antibody, and optionally the anti-Ki-67 comprises monoclonal mouse anti-Ki-67 clone MIB-1, or the anti-Ki-67 comprises a substantially isolated or substantially purified monoclonal mouse anti-Ki-67 clone MTB-1; or
(c) the total number of Ki-67 stained viable invasive tumor or cancer cells is evaluated under high magnification, and optionally the high magnification is at least about 10× magnification, or is between about 10× and 40× magnification, or is between about 10× and 60× magnification; or
(d) the invasive tumor or cancer cell is counted as positively stained with anti-Ki-67 if there is convincing and complete nuclear anti-Ki-67 staining and the invasive tumor cell and cancer cell is counted at an anti-Ki-67 nuclear staining at any intensity 1+ and higher; or
(e) there is convincing and complete nuclear anti-Ki-67 staining, and at an anti-Ki-67 nuclear staining at any intensity 1+ and higher, when:
(i) the staining signal is unequivocally brown, or
(ii) the staining corresponds to a nucleus, or
(iii) the staining covers the whole chromatin distribution within the nucleus, or
(iv) cells that exhibit a grey coloring in the nucleus are considered not stained with anti-Ki-67, or
(v) any combination of two or more of (i) to (iv); or
(f) there is convincing and complete nuclear anti-Ki-67 staining, and at an anti-Ki-67 nuclear staining at any intensity 1+ and higher, when:
(i) the staining signal is unequivocally brown,
(ii) the staining corresponds to a nucleus,
(iii) the staining covers the whole chromatin distribution within the nucleus, and
(iv) cells that exhibit a grey coloring in the nucleus are considered not stained with anti-Ki-67; or
(g) the method further comprises excluding from the calculation of the Ki-67 score (%): tumor cells or cancer cells with only cytoplasmic or membrane staining; non-invasive neoplasia or carcinoma in situ cells, non-viable or necrotic tumor or cancer cells, apoptotic nuclei or nuclear debris, tumor or cancer cells in poorly preserved tissue areas, benign epithelial cells, non-neoplastic cells and/or lymphocytes with nuclear staining, apoptotic cells, necrotic cells, cells not exhibiting an intended color, cells in which a stain reflecting of binding of the antibody to Ki-67 is not present throughout the entire chromatin distribution in the nucleus, cells exhibiting membrane staining, cells exhibiting cytoplasmic staining, lymphocytes, and stromal cells; or
(h) cells on an edge of a tissue sample specimen are not scored if staining due to an edge artifact is inconsistent with the rest of the tissue sample specimen; or
(i) in step (b) determining if the tissue section is or is not adequate for the determining and scoring of the amount of nuclear expression of protein Ki-67, wherein one parameter considered is: a tissue section is adequate for evaluation if about 200 or more viable invasive tumor cells are present, or
(j) wherein in step (d):
(i) if the Ki-67 Score (%) is less than (<) 20%, then the tissue sample is determined to have diagnostic negative Ki-67 expression; and
(ii) if the Ki-67 Score (%) is greater than or equal to (≥) 20%, then the tissue sample is determined to have diagnostic positive Ki-67 expression; or
(k) the tissue sample comprises a formalin-fixed, paraffin-embedded (FFPE) specimen; or
(l) the section of the tissue sample is prepared by a protocol comprising fixation in about 10% neutral buffered formalin for between about 6 to 72 hours; or
(m) the tumor or cancer is a breast cancer or breast carcinoma, a head and neck cancer, a colorectal cancer, a bladder cancer, a lung cancer, a gastrointestinal stromal tumor (GIST), a prostate cancer, a cervical cancer, or a renal cell carcinoma;
(n) the tumor or cancer is an invasive or metastatic breast carcinoma or breast cancer, or
(o) the tissue sample is a biopsy sample, or the tissue sample is or is derived from a needle biopsy sample, or the tissue sample is or is derived from a fine-needle aspirate, a cytology specimen or a bone decalcification.

14. A kit comprising an antibody which specifically binds to Ki-67, and scoring guidelines comprising a method of any of claims 1 to 13, or of any of the preceding claims; and optionally the kit comprises scoring guidelines, or further comprises images: indicating a plurality of Ki-67 staining levels or depicting staining of the whole nuclear chromatin distribution.

15. A method for determining and scoring the extent of nuclear expression of protein Ki-67 (also known as MK167) in cancer or tumor cells, the method comprising:
(a) staining cancer or tumor cells with an antibody which specifically binds to Ki-67; and
(b) determining the total number of viable invasive tumor or cancer cells having anti-Ki-67 nuclear staining, and determining the total number of staining and non-staining viable invasive tumor or cancer cells in at least a portion of the cancer or tumor cells,
wherein an invasive tumor or cancer cell is counted as positively stained with anti-Ki-67 if there is convincing and complete nuclear anti-Ki-67 staining, and if the invasive tumor or cancer cell displays at an anti-Ki-67 nuclear staining at any intensity above a defined threshold, and
(c) determining a Ki-67 score (%),
wherein the Ki-67 score (%) is the number of Ki-67 staining viable invasive tumor or cancer cells found in the at least a portion of the cancer or tumor cells divided by the total number of staining and non-staining viable invasive tumor or cancer cells, multiplied by 100.

16. A method for assessing the extent of Ki-67 expression comprising:
contacting a sample or a portion thereof comprising cancer or tumor cells from an individual with an antibody or portion thereof which specifically binds to Ki-67; and, determining a Ki-67 score (%) by dividing the number of Ki-67 staining viable tumor or cancer cells in the sample or portion thereof specifically bound by the antibody with the total number of staining and non-staining viable cancer or tumor cells and multiplying the result by 100, thereby obtaining the Ki-67 score (%).
